(19) United States
(12) Patent Application Publication
(10) Pub. No.: US 2025/0354150 A1
(43) Pub. Date: Nov. 20, 2025

(54) ALU SINES OF THE MIR-498(46) CISTRON MEDIATE INTRINSIC INTERFERON AND ANTIVIRAL RESPONSE IN HUMAN PLACENTA

(71) Applicant: UNIVERSITY OF SOUTH FLORIDA, Tampa, FL (US)

(72) Inventor: Hana TOTARY-JAIN, Wesley Chapel, FL (US)

(21) Appl. No.: 18/868,690

(22) PCT Filed: May 23, 2023
(86) PCT No.: PCT/US2023/067369

§ 371 Date: Nov. 22, 2024

Related U.S. Application Data
(60) Provisional application No. 63/344,846, filed on May 23, 2022.
(60) Provisional application No. 63/490,751, filed on Mar. 16, 2023.

(51) Int. Cl. C12N 15/113; A61P 31/04; C07K 14/555; C12Q 1/6809; C12Q 1/70

(52) U.S. Cl. CPC C12N 15/1131; A61P 31/04; C07K 14/555; C12Q 1/6809; C12Q 1/701; C12N 2310/141; C12Q 2600/106; C12Q 2600/178

(57) ABSTRACT

Described herein relates to novel methods for determining susceptibility for infection and/or disease (e.g., pregnancy complication and/or cancer), and/or predicting severity of infection and/or disease by measuring circulating Alu RNA by RT-PCR (e.g., circulating blood, serum, and/or plasma). Additionally, described herein relates to novel methods of treating infection and/or disease (e.g., pregnancy complication and/or cancer), via increasing immune response, optimizing vaccine delivery, via administering at least one Alu RNA into the subject.

### CROSS-REFERENCE TO RELATED APPLICATIONS

This nonprovisional application claims the benefit of U.S. Provisional Application No. 63/344,846 entitled “ALU SINES OF THE MIR-498 CISTRON MEDIATE INTRINSIC INTERFERON AND ANTIVIRAL RESPONSE IN HUMAN PLACENTA” filed May 23, 2022 by the same inventor, and U.S. Provisional Application No. 63/490,751 entitled “METHOD FOR MEASURING ALU RNA” filed Mar. 16, 2023, all of which are incorporated herein by reference, in their entireties, for all purposes.

### FEDERALLY SPONSORED RESEARCH OR DEVELOPMENT

This invention was made with Government support under Grant No. NIH HL128411 awarded by the National Institutes of Health. The government has certain rights in the invention.

### BACKGROUND OF THE INVENTION

### 1. Field of the Invention

This invention relates to methods of treating infection. Specifically, the invention provides novel methods of treating infection and/or disease (e.g., pregnancy complication and/or cancer), via increasing antiviral response and/or immune response. Additionally, the invention provides novel methods of optimizing vaccine delivery, determining susceptibility for infection and/or disease (e.g., pregnancy complication and/or cancer), and/or predicting severity of infection and/or disease (e.g., pregnancy complication and/or cancer) by measuring circulating Alu RNA by RT-PCR.

### 2. Brief Description of the Prior Art

Viral infections, bacterial infection, and/or parasitic infection have often caused devastating effects on pregnancy outcome, fetal development, maternal health, and/or the average health of an individual. For example, since the defense against pathogens during pregnancy conflicts with the tolerance to the allogenic fetus, the placenta at the maternal-fetal interfaces has developed unique antiviral defense mechanisms. Unlike somatic cells that require pathogen-associated molecular patterns (PAMPs) to mediate IFN induction, human syncytiotrophoblasts constitutively produce type III IFNs, even in the absence of an infection, by unknown mechanism.

In this manner, the evolution of invasive placentation in primates coincided with the emergence of mir-498 cistron (known as C19MC), including mir-498 (46) cistron. For example, the primate-specific cluster contains 46 highly homologous miRNA precursor sequences flanked by Alu retrotransposons (RTs) that have mediated its rapid expansion. Trophoblast-derived exosomes containing specific miRNAs of the mir-498 (46) cistron attenuate viral replication by inducing autophagy in recipient cells. However, the role and effect of the Alu RTs and/or Alu RNA in an adult individuals, pregnant and/or postpartum individuals and/or fetuses, which are constitutively transcribed with the mir-498 cistrons, has not been investigated.

Accordingly, what is needed is novel methods of treating infection and/or disease (e.g., pregnancy complication and/or cancer), via increasing antiviral response and/or immune response, in addition to novel methods of optimizing vaccine delivery, determining susceptibility for infection and/or disease (e.g., pregnancy complication and/or cancer), and/or predicting severity of infection and/or disease (e.g., pregnancy complication and/or cancer) by measuring circulating Alu RNA by RT-PCR (e.g., circulating blood, serum, and/or plasma). However, in view of the art considered as a whole at the time the present invention was made, it was not obvious to those of ordinary skill in the field of this invention how the shortcomings of the prior art could be overcome.

### SUMMARY OF THE INVENTION

The long-standing but heretofore unfulfilled need, stated above, is now met by a novel and non-obvious invention disclosed and claimed herein. In an aspect, the present disclosure pertains to a method of predicting susceptibility for infection in a subject. In an embodiment, the method may comprise the following: (a) obtaining an expression level of at least one Alu RNA and/or at least one miR member in a sample suspected of comprising the infection; (b) determining a ratio of Alu RNA:miR member within the sample; (c) obtaining an expression level of Alu RNA and miR members within a control, such that control ratio of Alu RNA:miR members may be determined; and (d) comparing the Alu RNA:miR member ratio of the sample suspected of comprising the infection to the Alu RNA:miR member control ratio, such that when the ratio comprises a higher expression level of the at least one Alu RNA as compared to the expression level of the at least one miR member within the sample suspected of comprising the infection as compared to the control ratio, the high expression level of the at least one Alu RNA may be indicative of a high susceptibility for the infection.

In some embodiments, the infection may include but is not limited to a VSV, RSV, SARS-CoV2, and/or Zika virus. Additionally, in some embodiments, the at least one Alu RNA may be configured to express at least one antiviral biological product, the antiviral biological product being configured to increase an antiviral response within the sample suspected of comprising the infection. As such, in these other embodiments, the at least one biological product may comprise but is not limited to a C19MC, an IFN, an Ifnl2, an Ifnl3, and/or an ISG. In this manner, the INF may comprise a type III interferon. In some embodiments, subsequent to administering the mir-498 and/or the ALU RNA portion thereof, at least one Alu RNA may be configured to generate the at least one antiviral biological product intrinsically.

Moreover, another aspect of the present disclosure pertains to predicting severity of infection in a subject. In an embodiment, the method may comprise the following: (a) obtaining an expression level of at least one Alu RNA in a sample suspected of comprising the infection; (b) obtaining a viral burden in the sample suspected of comprising the infection; (c) determining a ratio of Alu RNA:Viral Burden; (d) obtaining an expression level of Alu RNA and/or a viral burden of a control, such that a control ratio of Alu RNA to viral burn may be determined; (e) comparing Alu RNA:Viral Burden of the sample suspected of comprising the infection to the Alu RNA:Viral Burden control ratio, such that when the ratio comprises a lower expression level of Alu RNA to a higher viral burden for the sample suspected of comprising the infection as compared to the control ratio, the Alu RNA:Viral Burden of the sample is indicative of a high severity of the infection; and (f) administering at least one additional mir-498 cistron and/or Alu RNA portion, in the forward direction and/or the reverse direction, thereof if a lower expression level of Alu RNA to a higher viral burden is obtained within the sample.

In some embodiments, the infection may include but is not limited to a VSV, RSV, SARS-CoV2, and/or Zika virus. In some embodiments, the at least one Alu RNA of the mir-498 cistron is embedded in the sense strands and/or antisense strands of the mir-498 cistron. Additionally, in some embodiments, the at least one Alu RNA may be configured to express at least one antiviral biological product, the antiviral biological product being configured to increase an antiviral response within the sample suspected of comprising the infection. As such, in these other embodiments, the at least one biological product may comprise but is not limited to a C19MC, an IFN, an Ifnl2, an Ifnl3, and/or an ISG. In this manner, the INF may comprise a type Ill interferon. In some embodiments, subsequent to administering the mir-498, the at least one Alu RNA of the mir-498 may be configured to generate the at least one antiviral biological product intrinsically.

Furthermore an additional aspect of the present disclosure pertains to a method of treating an infection in a subject. In an embodiment, the method may comprise the following: (a) obtaining a sample suspected of comprising the infection from the subject; (b) obtaining an expression level of at least one at least one Alu RNA from the sample; (c) calculating a median expression level using a highest and a lowest value of the expression levels of the at least one at least one Alu RNA, biological product; (d) determining if the infection of the subject will be sensitive to at least one additional Alu RNA of a miR-498 cistron by comparing the expression level of the sample suspected of comprising the infection to the median expression level, such that a low expression level as compared to the median expression level may be indicative of the infection; and (f) administering at least mir-498 cistron and/or Alu RNA portion, in the forward direction and/or the reverse direction, thereof to the subject having the low expression level score, such that subsequent to receiving the at least one mir-498 cistron and/or Alu RNA portion thereof, the sample is configured to increase production of at least one antiviral biological product, optimizing an antiviral response of the at least one cell of the subject.

In some embodiments, the infection may include but is not limited to a VSV, RSV, SARS-CoV2, and/or Zika virus. In some embodiments, the at least one Alu RNA of the miR-498 cistron is embedded in the sense strands, antisense strands, or both of the miR-498 cistron. In this manner, in these other embodiments, the method may further comprise the step of, after administering the at least one addition miR-498 cistron to the subject, generating the at least one Alu RNA, such that the at least one Alu RNA may be configured to generate the at least one antiviral biological product. Furthermore, the at least one antiviral biological product generated may comprise, but is not limited to a C19MC, an IFN, an Ifnl2, an Ifnl3, and/or an ISG. Additionally, the INF may comprise a type Ill interferon. In some embodiments, the at least one Alu RNA may be configured to generate the at least one antiviral biological product intrinsically.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not restrictive.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1 is an image of the activated cistron, according to an embodiment of the present disclosure.

FIGS. 2A-2H are a series of images depicting transcriptional activation of the mir-498 (46) cistron induced a strong type III IFNs response, according to an embodiment of the present disclosure. FIG. 2A is an image depicting both an upregulated miRNA and a downregulated miRNA of the merged miR members (e.g., GFP, BB, and/or 759) of the mir-498 (46) cistron.

FIG. 2B is a graph depicting a relative expression of the mir-498 (46) cistron. FIG. 2C is a plot depicting an enrichment score for the mir-498 (46) cistron. FIG. 2D is an image depicting a GOBP Defense Response to a virus for the mir-498 (46) cistron. FIG. 2E is an image depicting a representative agarose gel of IFNL3 and GAPDH RT-PCR expression within of the mir-498 (46) cistron. FIG. 2F is a graph depicting the IFNL 1/3 value for the merged miR members of the mir-498 (46) cistron. FIG. 2G is a graph depicting the relative IRF7 expression for the merged miR members of the mir-498 (46) cistron. FIG. 2H is a graph depicting a relative expression of a gene for the merged miR members of the mir-498 (46) cistron.

FIGS. 3A-3B are a series of images depicting the relationship between mir-498 (46) cistron and IFNL3 and antiviral response genes, according to an embodiment of the present disclosure. FIG. 3A is an image depicting a representative agarose gel of IFNL3 and GAPDH RT-PCR of the mir-498 (46) cistron for HTR-8/SVneo cells. FIG. 3B is a graph depicting a relative expression of a gene for BB and 759 of the mir-498 (46) cistron. HTR-8/SVneo cells, which are immortalizing human extra villous trophoblasts that do not express the mir-498 (46) cistron, were used.

FIGS. 4A-4J are a series of images depicting mir-498 (46) cistron activation induced type III IFN, ISGs, and antiviral protection in a microRNA-independent mechanism, according to an embodiment of the present disclosure. FIG. 4A is a graph and representative agarose gel depicting a relative miR-517 expression of BB and 759 of the mir-498 (46) cistron. FIG. 4B is an image depicting both an upregulated miRNA and a downregulated miRNA of merged miR members of the mir-498 (46) cistron. FIG. 4C is a plot depicting an enrichment score of 293T cells and NoDice 2-20 cells comprising the miR members of the mir-498 (46) cistron. FIG. 4D is a table depicting a GODP-negative regulation of viral process within 293T cells and a NoDice 2-20 cells. FIG. 4E is an image depicting a representative agarose gel depicting a IFNL3 and GAPDH RT-PCR expression within of the mir-498 (46) cistron within a 293T cell and a 2-20 cell. FIG. 4F is a graph depicting an amount of IFNLE amount of a merged miR member of the mir-498 (46) cistron within a 293T cell and a 2-20 cell. FIG. 4G is a graph depicting a relative expression of a gene within a merged miR member of the mir-498 (46) cistron within a 293T cell and a 2-20 cell. FIG. 4H is a graph depicting a relative viral RNA % control within a merged miR member of the mir-498 (46) cistron within a 293T cell. FIG. 4I is a graph depicting a relative viral RNA % control within a merged miR member of the mir-498 (46) cistron within a 2-20 cell. FIG. 4J a graph depicting a relative viral RNA % control within a merged miR member of the mir-498 (46) cistron within a HTR8/SVneo cell.

FIGS. 5A-5C are a series of images depicting mir-498 (46) cistron activation induced type III IFN, ISGs, and antiviral protection in a microRNA-independent mechanism, according to an embodiment of the present disclosure. FIG. 5A is an image depicting a representative agarose gel of GAPDH and DROSHA within kDA, 293T, and DROSHA ko. FIG. 5B is an image depicting an agarose gel of GAPDH and IFNL3 expression in a microRNA-independent mechanism. FIG. 5C is a graph depicting a relative expression of a gene within a microRNA-independent mechanism of the miR-498 (46) cistron.

FIG. 6 is an image depicting activation of the miR-498 (46) cistron increase Alu dsRNA that mediate intrinsic type III IFN production and antiviral protection, according to an embodiment of the present disclosure.

FIGS. 7A-7I are a series of images depicting transcriptional activation of miR-498 (46) cistron increases dsRNA that mediate the intrinsic type III IFNs production and antiviral protection, according to an embodiment of the present disclosure. FIG. 7A is a graph that depicts relative expression of a gene within a merged miR member of miR-498 (46) cistron. FIG. 7B is an image depicting a representative agarose gel of gene expression within a HeLA 293T and a HeLA 2-20 cell. FIG. 7C is a graph depicting a relative fl-Alu/SC-Alu ratio within a HeLA cell, a 293T cell, and a 2-20 cell. FIG. 7D is a graph and image depicting a relative intensity of a merged miR member of the miR-498 (46) cistron within a 293T cell and a 2-20 cell. FIG. 7E is an image depicting an agarose gel of gene expression within a HTR8/SVneo cell. FIG. 7F is a graph depicting a relative expression of a gene within the miR-498 (46) cistron. FIG. 7G is a graph depicting a relative viral RNA (% control) within a HTR8/SVneo comprising a miR-498 (46) cistron. FIG. 7H is a graph depicting a relative viral RNA (% control) within a 293T cell+VSV comprising a miR-498 (46) cistron. FIG. 7I is a graph depicting a titer (PFU/mL) within a 293T cell+VSV comprising a miR-498 (46) cistron.

FIGS. 8A-8E are a series of images depicting expression of mir-498 (46) miRNAs localized with Alu transcripts and dsRNA in placental villous trophoblast, according to an embodiment of the present disclosure. FIG. 8A is an image depicting expression of mir-498 (46) miRNAs localized with Alu transcripts and dsRNA in a human placenta (term). FIG. 8B is an image depicting expression of mir-498 (46) miRNAs localized with Alu transcripts and dsRNA in in a human placenta (1^{st} trimester). FIG. 8C is an image depicting an agarose gel of gene expression of mir-498 (46) miRNAs localized with Alu transcripts and dsRNA in placental villous trophoblast within a HeLA cell and a human cell. FIG. 8D is an image depicting DAPI and J2 gene expression within a human placenta (term). FIG. 8E is a graph depicting a relative fold change of a gene expression within a PL cell and an LV cell of a human.

FIGS. 9A-9B are a series of images depicting ALU and GAPDH RT-PCR, according to an embodiment of the present disclosure. FIG. 9A is an image depicting a representative agarose gel of Alu and GAPDH RT-PCR and a control ALU PCR product using total RNA. FIG. 9B is a graph depicting a densitometric quantification of fl-ALU to sc-ALU ratio normalized to GAPDH.

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that one skilled in the art will recognize that other embodiments may be utilized, and it will be apparent to one skilled in the art that structural changes may be made without departing from the scope of the invention. Elements/components shown in diagrams are illustrative of exemplary embodiments of the disclosure and are meant to avoid obscuring the disclosure. Any headings, used herein, are for organizational purposes only and shall not be used to limit the scope of the description or the claims. Furthermore, the use of certain terms in various places in the specification, described herein, are for illustration and should not be construed as limiting.

Reference in the specification to “one embodiment,” “preferred embodiment,” “an embodiment,” or “embodiments” means that a particular feature, structure, characteristic, or function described in connection with the embodiment is included in at least one embodiment of the disclosure and may be in more than one embodiment. The appearances of the phrases “in one embodiment,” “in an embodiment,” “in embodiments,” “in alternative embodiments,” “in an alternative embodiment,” or “in some embodiments” in various places in the specification are not necessarily all referring to the same embodiment or embodiments. The terms “include,” “including,” “comprise,” and “comprising” shall be understood to be open terms and any lists that follow are examples and not meant to be limited to the listed items.

### Definitions

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

The term “about”, “approximately”, or “roughly” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. As used herein “about” refers to within +15% of the numerical.

As used herein, the term “comprising” is intended to mean that the products, compositions and methods include the referenced components or steps, but not excluding others. “Consisting essentially of” when used to define products, compositions and methods, shall mean excluding other components or steps of any essential significance. Thus, a composition consisting essentially of the recited components would not exclude trace contaminants and pharmaceutically acceptable carriers. “Consisting of” shall mean excluding more than trace elements of other components or steps.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include the individual values and sub-ranges within the indicated range. Thus, included in this numerical range are individual values such as 2, 3, and 4 and sub-ranges such as from 1-3, from 2-4 and from 3-5, etc. This same principle applies to ranges reciting only one numerical value. Furthermore, such an interpretation should apply regardless of the range or the characteristics being described

All numerical designations, including ranges, are approximations which are varied up or down by increments of 1.0, 0.1, 0.01 or 0.001 as appropriate. It is to be understood, even if it is not always explicitly stated, that all numerical designations are preceded by the term “about”. It is also to be understood, even if it is not always explicitly stated, that the compounds and structures described herein are merely exemplary and that equivalents of such are known in the art and can be substituted for the compounds and structures explicitly stated herein.

“Patient” or “subject” is used to describe an animal, preferably a human (e.g., a pregnant woman, an individual suspected of having cancer, and/or an immunocompromised individual), to whom treatment is administered, including prophylactic treatment with the compositions of the present invention.

The term “biomarker” is used herein to refer to a molecule whose level of nucleic acid or protein product has a quantitatively differential concentration or level with respect to an aspect of a biological state of a subject. The level of the biomarker can be measured at both the nucleic acid level as well as the polypeptide level. At the nucleic acid level, a nucleic acid gene or a transcript which is transcribed from any part of the subject's chromosomal and extrachromosomal genome, including for example the mitochondrial genome, may be measured. Preferably an RNA transcript, more preferably an RNA transcript includes a primary transcript, a spliced transcript, an alternatively spliced transcript, or an mRNA of the biomarker is measured. At the polypeptide level, a prepropeptide, a propeptide, a mature peptide or a secreted peptide of the biomarker may be measured. A biomarker can be used either solely or in conjunction with one or more other identified biomarkers so as to allow correlation to the biological state of interest as defined herein.

The term “biological state” as used herein refers to the result of the occurrence of a series of biological processes. As the biological processes change relative to each other, the biological state also changes. One measurement of a biological state is the level of activity of biological variables such as biomarkers, parameters, and/or processes at a specified time or under specified experimental or environmental conditions. A biological state can include, for example, the state of an individual cell, a tissue, an organ, and/or a multicellular organism. A biological state can be measured in samples taken from a normal subject or a diseased subject thus measuring the biological state at different time intervals may indicate the progression of a disease in a subject. The biological state may include a state that is indicative of disease (e.g. diagnosis); a state that is indicative of the progression or regression of the disease (e.g. prognosis); a state that is indicative of the susceptibility (risk) of a subject to the disease; and a state that is indicative of the efficacy of a treatment of the disease. In some embodiments the disease is a viral infection, a bacterial infection, and/or a parasitic infection.

The term “baseline level” or “control level” of biomarker expression or activity refers to the level against which biomarker expression in the test sample can be compared. In some embodiments, the baseline level can be a normal level, meaning the level in a sample from a normal patient. This allows a determination based on the baseline level of biomarker expression or biological activity, whether a sample to be evaluated for the disease (e.g., pregnancy complication and/or cancer) and/or infection (e.g. viral infection) has a measurable increase, decrease, or substantially no change in biomarker expression as compared to the baseline level. The term “negative control” used in reference to a baseline level of biomarker expression generally refers to a baseline level established in a sample from the subject or from a population of individuals which is believed to be normal. In other embodiments, the baseline level can be indicative of a positive diagnosis of disease (e.g. positive control). The term “positive control” as used herein refers to a level of biomarker expression or biological activity established in a sample from a subject, from another individual, or from a population of individuals, where the sample was believed, based on data from that sample, to have the disease (e.g., pregnancy complication and/or cancer) and/or infection (e.g. viral infection). In other embodiments, the baseline level can be established from a previous sample from the subject being tested, so that the disease progression or regression of the subject can be monitored over time and/or the efficacy of treatment can be evaluated.

The genes of the present invention may serve as biomarkers for: (1) the diagnosis of disease; (2) the prognosis of diseases (e.g. monitoring disease progression or regression from one biological state to another; (3) the determination of susceptibility or risk of a subject to disease; or (4) the evaluation of the efficacy to a treatment for disease (e.g., pregnancy complication and/or cancer) and/or infection (e.g. viral infection). For the diagnosis of disease, the level of the specific circulating biomarkers in the subject can be compared to a baseline or control level in which if the level is above the control level, a certain disease is implicated whereas if the level is below the control level, a different disease is implicated. The prognosis of disease can be assessed by comparing the level of the specific biomarker at a first timepoint to the level of the biomarker at a second timepoint which occurs at a given interval after the first timepoint. The evaluation of the efficacy of the treatment for a disease can be assessed by comparing the level of the specific biomarker at a first timepoint before administration of the treatment to the level of the biomarker at a second timepoint which occurs at a specified interval after the administration of the treatment.

The term “expression level” as used herein refers to detecting the amount or level of expression of a biomarker of the present invention. The act of actually detecting the expression level of a biomarker refers to the act of actively determining whether a biomarker is expressed in a sample or not. This act can include determining whether the biomarker expression is upregulated, downregulated or substantially unchanged as compared to a control level expressed in a sample. The expression level in some cases may refer to detecting transcription of the gene encoding a biomarker protein and/or to detecting translation of the biomarker protein.

Expression of genes/transcripts and/or polypeptides encoded by the genes represented by the biomarkers of the present invention can be measured by any of a variety of methods known in the art. In general, expression of a nucleic acid molecule (e.g. RNA or DNA) can be detected by any suitable method or technique of measuring or detecting gene or polynucleotide sequence or expression. Such methods include, but are not limited to, polymerase chain reaction (PCR), reverse transcriptase PCR (RT-PCR), in situ PCR, quantitative PCR (q-PCR), in situ hybridization, Southern blot, Northern blot, sequence analysis, microarray analysis, detection of a reporter gene, or any other DNA/RNA hybridization platforms.

The term “quantifying” or “quantitating” when used in the context of quantifying transcription levels of a gene can refer to absolute or relative quantification. Absolute quantification can be achieved by including known concentration(s) of one or more target nucleic acids and referencing the hybridization intensity of unknowns with the known target nucleic acids (e.g. through the generation of a standard curve). Alternatively, relative quantification can be achieved by comparison of hybridization signals between two or more genes, or between two or more treatments to quantify the changes in hybridization intensity and, by implication transcription level.

“Sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof.

The term “nucleic acid” as used herein may be double-stranded, single-stranded, or contain portions of both double and single stranded sequence. If the nucleic acid is single-stranded, the sequence of the other strand is also identifiable and thus the definition includes the complement of the sequence disclosed.

Methods to measure protein/polypeptide expression levels of selected biomarkers in the present invention may include, but are not limited to: Western blot, immunoblot, enzyme-linked immunosorbent assay (ELISA), radioimmunoassay (RIA), immunoprecipitation, surface plasmon resonance, chemiluminescence, fluorescent polarization, phosphorescence, immunohistochemical analysis, liquid chromatography mass spectrometry (LC-MS), matrix-assisted laser desorption/ionization time-of-flight (MALDI-TOF), mass spectrometry, microcytometry, microarray, microscopy, fluorescence activated cell sorting (FACS), flow cytometry, and assays based on a property of the protein including but not limited to DNA binding, ligand binding, or interaction with other protein partners.

The terms “overexpression” and “underexpression” as used herein refers to the expression of a gene of a patient at a greater or lesser level, respectively, than the normal or control expression of the gene, as measured by gene expression product expression such as mRNA or protein expression, in a sample that is greater than the standard of error of the assay used to assess the expression. A “significant” expression level may be a level which either meets or is above or below a predetermined score for a gene

Wherever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Wherever the term “no more than,” “less than,” or “less than or equal to” precedes the first numerical value in a series of two or more numerical values, the term “no more than,” “less than” or “less than or equal to” applies to each of the numerical values in that series of numerical values. For example, less than or equal to 1, 2, or 3 is equivalent to less than or equal to 1, less than or equal to 2, or less than or equal to 3.

Infection Susceptibility and/or Severity Detection

The human genome contains roughly 1 million copies of Alu SINEs embedded in the positive and the negative strands of the genomic DNA near and/or within coding and/or non-coding genes, which can be transcribed by RNA Pol II. As such, Alu SINEs contain internal RNA Pol III promoters, and under stressful conditions, Alu SINEs may be transcribed independently to produce short lived full length Alu (hereinafter “fl-Alu”) transcripts, which may be processed into a stable small cytoplasmic Alu (hereinafter “sc-Alu”) RNA. In some embodiments, the sc-Alu RNA may be double stranded

Additionally, Alu SINEs are highly homologous repetitive elements that constitute 11% of the human genome. Therefore, detection and/or comparative quantification of Alu RNA expression levels is technically challenging. It was found that transcriptional activation of a mir-498 cistron (e.g., mir-498 (46)), even in the absence of infection (e.g., viral infection), induces type III interferon (hereinafter “IFN”), and/or its downstream interferon stimulated genes (hereinafter “ISGs”) and antiviral response genes. It is further shown herein roughly 50% of the mir-498 cistron consist of the highly homologous Alu repeats embedded in both the sense and/or antisense strands. These findings illuminate a previously unknown pathway that transcriptional activation of the mir-498 cistron generates Alu double stranded (ds) RNA, such as sc-Alu RNA, (hereinafter “Alu RNA”), which are responsible for the intrinsic induction of type III IFN and the antiviral response.

As such, the present disclosure pertains to novel methods of treating infection (e.g., viral infection) and/or disease (e.g., pregnancy complication and/or cancer), via increasing antiviral response and/or immune response. Additionally, the invention provides novel methods of optimizing vaccine delivery, determining susceptibility for infection (e.g. viral infection) and/or disease (e.g., pregnancy complication and/or cancer), and/or predicting severity of infection and/or disease (e.g., pregnancy complication and/or cancer) by measuring circulating Alu RNA by RT-PCR (e.g., circulating blood, serum, and/or plasma). As used herein, the term “pregnancy complication” may refer to any physical and/or mental condition that may affect the health of the pregnant or postpartum subject and/or the baby known in the art. The pregnancy complication may be preeclampsia, teratogenic effects, such as birth defects microcephaly, hearing loss, ocular abnormalities, and/or hepatosplenomegaly, and/or mis carriage. For ease of reference, the exemplary embodiment described herein refers to teratogenic effects, but this description should not be interpreted as limiting to other pregnancy complications.

In an embodiment, the method may include the step of selecting a subject with the infection, or a risk for contracting an infection, and determining the amount of circulating Alu SINEs. As such, in this embodiment, the method may also include administering to the subject a therapeutically effective amount of at least one mir-498 cistron comprising the highly homologous Alu repeats in the forward direction and/or reverse direction (i.e., ALU RNA and/or ALU (ds) RNA). In this manner, in these other embodiments, the nucleic acid molecule may comprise any vector known in the art, including but not limited to a plasmid vector, a viral vector, and/or an in vitro transcribed Alu RNA that contain modified nucleotides.

In addition, in this embodiment, the infection may comprise a viral infection. The viral infection may be caused by any type of virus known in the art. For example, in some embodiments, the virus may be any RNA virus known in the art. The RNA virus may be Vesicular stomatitis virus (hereinafter “VSV”), Zika Virus, and/or respiratory syncytial virus (hereinafter “RSV”), and/or SARS-CoV2 virus. For ease of reference, the exemplary embodiment described herein refers to Zika Virus, but this description should not be interpreted as exclusionary of other RNA viruses. Moreover, in some embodiments, the virus may be any DNA virus known in the art. The DNA virus may be vaccinia virus, herpes simplex viruses (HSV-1 and -2), Epstein-Ban virus, and hepatitis B virus. For ease of reference, the exemplary embodiment described herein refers to herpes simplex viruses, but this descriptions should not be interpreted as exclusionary of other DNA viruses. In some embodiments, the virus may be cytomegalovirus (CMV).

In an embodiment, the infection may comprise a bacterial infection. The bacterial infection may be caused by any type of infection-inducing bacteria known in the art. For example, in some embodiments, the bacteria may be Listeria monocytogenes, Staphylococcus aureus, Streptococcus, Burkholderia pseudomallei, Helicobacter pylori, and/or Vibrio cholerae.

In some embodiments, the microbial infection is a parasitic infection (e.g., fungal infection). The parasitic infection may be caused by any infection-inducing parasite known in the art. For example, in some embodiments, the parasite may comprise the following: Toxoplasma Gondii. Candida, Cryptococcus, Aspergillus, Histoplasma capsulatum, Coccidioides immitis, C. posadasii, Blastomyces dermatitidis and/or Pneumocystis jirovecii.

In an embodiment, the at least one mir-498 cistron and/or the nucleic acid molecule encoding the at least one mir-498 cistron miRNA and/or the at least one Alu RNA, transcribed in the forward direction and/or reverse direction, thereof, may be administered prophylactically to prevent infection (e.g., viral infection). In other examples, the at least one mir-498 cistron miRNA and/or the nucleic acid molecule encoding the at least one mir-498 cistron miRNA and/or the at least one Alu RNA, transcribed in the forward direction and/or reverse direction, thereof, may be administered to treat an existing infection.

In an embodiment, at least one Alu RNA, transcribed in the forward direction and/or reverse direction, of the mir-498 cistron may be administered to the subject. Similarly, if the subject is administered a nucleic acid molecule comprising the mir-498 cistron and/or the Alu RNA portion thereof, the subject can be administered the entire mir-498 cistron and/or at least one portion that encodes the at least Alu RNA of the mir-498 cistron.

In an embodiment, at least one nucleotide sequences of at least one Alu RNA of the mir-498 cistron is at least 90%, at least 91%, at least 92%, at least 93%, at least 94%, at least 95%, at least 96%, at least 97%, at least 98%, and/or at least 99% identical to at least one SEQ ID NO: 1-78. In some embodiments, the at least one Alu RNA of the mir-498 (46) cistron may comprise at least one of SEQ ID NO: 1-78.

In addition, in an embodiment, the method may comprise at least one in vitro aspect. As such, in the embodiment, the cell may be a primary cell. In some embodiments, the cell is an immortalized cell.

Moreover, in an embodiment, the method may comprise at least one in vivo aspect, such that the mir-498 cistron may be administered to the subject, via contacting the cell with an effective amount of one or more miRs of the mir-498 cistron and/or a nucleic acid molecule encoding the mir-498 cistron and/or the Alu RNA portion thereof.

Additionally, in an embodiment, the at least one Alu RNA of the miR-498 cistron and/or the nucleic acid molecule encoding the miR-498 cistron and/or the Alu RNA portion thereof, may be configured to be administered to the subject and/or contacted with the cell using at least one liposomal formulation, cationic lipid and/or polypeptide carrier known in the art.

Furthermore, in an embodiment, an expression level of at least one Alu may be detected, in addition to at least on miR member of the mir-498 cistron, via at least one blood, serum, and/or plasma examination known in the art. The examination may be a plasma blood test, a serum blood test, a comprehensive metabolic panel, a basic metabolic panel, a blood enzyme test, a blood clotting test, a C-reactive protein (CRP) test, a erythrocyte sedimentation rate (ESR) test, and/or a plasma viscosity (PV) test. For ease of reference, the exemplary embodiment described herein refers to a plasma viscosity test, but this description should not be interpreted as limiting to other blood tests and/or examinations.

In this manner, in this embodiment, subsequent to the subject receiving a sample from at least one blood examination, the expression level of at least one Alu RNA and/or at least miR member of the mir-498 cistron may be determined, such that a Alu RNA:miR member ratio (hereinafter “Alu:miR”) (e.g., a 1:1 ratio) may be determined. As such, in an embodiment, when the Alu:miR ratio comprises an equal Alu RNA expression to miR member expression, the sample may be indicative of a normal condition within the sample. Accordingly, when the Alu:miR ratio comprises a high Alu RNA expression to the miR member expression, the Alu:miR ratio within the sample may be indicative of a infection (e.g., viral infection), since as known in the art, during infection, the expression of Alu RNA is increased but not the miR members of mir-498 does not change.

In addition, in an embodiment, when the expression level of the sample comprises a high Alu RNA count, the Alu RNA count within the sample may be indicative of a high antiviral and/or immune protection. In this same manner, when the expression level of the sample comprises a high Alu RNA count and/or a high level of viral burden, the Alu RNA:Viral Burden ratio may be indicative of an antiviral response and/or an immune response to the infection (e.g., viral infection). However, in this embodiment, when the expression level of the sample comprises a high Alu RNA count and/or a low viral burden, such that the Alu RNA:Viral Burden ratio within the sample may be indicative of a complication and/or disease. For example, with respect to mir-498 (46), when the expression level of the viral burden is low and the Alu RNA count is high within a sample, the Alu RNA count within the sample may be indicative of a pregnancy complication, such as preeclampsia, teratogenic effects such as birth defects microcephaly, hearing loss, ocular abnormalities, and/or hepatosplenomegaly.

Moreover, in this embodiment, when the expression level of the sample comprises the low Alu RNA count, the Alu RNA count within the sample may be indicative of a higher susceptibility to the infection (e.g., viral infection) as the antiviral and/or immune response is low, such that, in the same manner, when the expression level of the sample comprises a high Alu RNA count, the susceptibility to the infection (e.g., viral infection) is substantially decreased as the high level of Alu RNA intrinsically generates IFN production (e.g., type III IFN), causing an antiviral protection response. In addition, having the expression level of the sample comprising a high Alu RNA may be indicative of a low severity of infection, due to the immediate increased antiviral and/or immune response. As such, in some embodiments, with respect to an immunocompromised subject, when at least one mir-498 cistron comprising highly homologous Alu repeats (i.e., Alu RNA) is administered, the increase in the expression level of Alu RNA may optimize IFN production, increasing the autoimmune response within the immunocompromised subject, such that the increased Alu RNA expression level may be indicative of at least one cell comprising the mir-498 cistron and/or the Alu RNA of the mir-498 cistron fighting the infection within the subject. In this manner, having the expression level of the sample comprising the low Alu RNA count may indicate an increased severity and/or susceptibility of infection within the subject, as the antiviral response to the infection has not increased and/or is not currently active, allowing the viral burden of the infection to increase.

Furthermore, in an embodiment, with respect to subject suspected of comprising cancer; when the expression level of the sample comprises a high Alu RNA count, in addition to a high miR member expression, the Alu RNA count within the sample may be indicative of cancer within the subject, Alu-mediated immunomodulation response has been activated to protect the tumor from surrounding immune system response.

In an embodiment, the at least one Alu RNA may be configured to be compatible with at least one vaccine. In this manner, in an embodiment, when the vaccine is administered with the at least one Alu RNA transcribed in the forward direction and/or reverse direction, the at least one Alu RNA may be configured to increase IFN product and/or ISG production. As such, the increased IFN and/or ISG production creates an antiviral protection around the administered vaccine, such that the immune response may not attack and/or negatively react to the administered vaccine. Accordingly, in this embodiment, vaccine administration and/or incorporation within the subject may be optimized due to the suppressed immune response towards the vaccine, via the increased IFN and/or ISG product of the at least one Alu RNA.

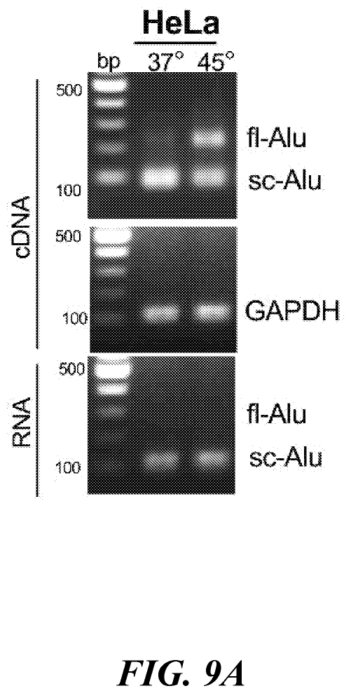

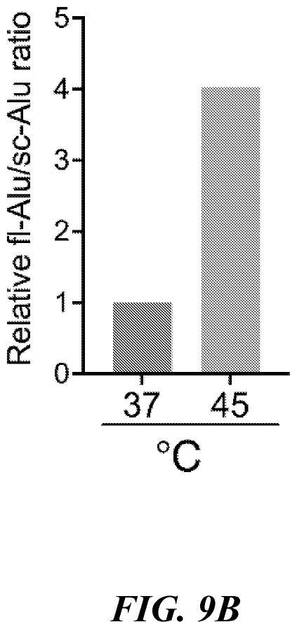

In an embodiment, in order to ensure the robustness of the method the following controls may be implemented: (1) after extensive treatment of the total RNA with DNase, in order to exclude the potential contamination of the RNA with genomic DNA, performing the Alu PCR using the same primer set, as shown in TABLE 1, on the total RN, as shown in FIG. 9B; and (2) to confirm that the competitive RT-PCR is indeed specific to the Alu SINEs, cloning and/or sequencing the fl-Alu RT-PCR products of heat shocked Hela cells and/or all these clone sequences aligned to Alu SINEs, as shown in TABLE 3.

As such, in an embodiment, the method may incorporate and/or use at least one primer set designed to detect the levels of fl- and/or sc-Alu RNA within the cells irrespective of their transcription by RNA Pol II and/or Pol III. Moreover, in this embodiment, the levels of the fl- and/or sc-Alu RNA may be based on the competition on the primers. In this manner, the most abundant form of Alu RNA may then compete and/or be amplified more than the less abundant form. For example, in some embodiments, under steady state conditions, such as Hela cells at 37° C., most of the endogenous Alu RNA may be processed into sc-Alu, such that the competitive Alu RT-PCR may show a very low fl-Alu to sc-Alu ratio, as shown in FIG. 9A and FIG. 9B. However, in these other embodiments, upon heat shock exposure that induce the fl-Alu transcription by RNA Pol III, the fl-Alu may compete on the primers and/or the fl-Alu may be configured to amplify more than the sc-Alu, such that the fl-Alu to sc-Alu ratio may increase, as shown in FIG. 9A and FIG. 9B.

Administration of miR-498 Cistron

In an embodiment, the nucleic acid molecule encoding the miR-498 cistron and/or the Alu RNA portion, transcribed in a forward direction and/or a reverse direction, thereof may be administered to the subject in need of treatment using any suitable means known in the art. As such, the nucleic acid-based therapeutic agents may be administered to a subject by any suitable route. Additionally, in this embodiment, the nucleic acid molecules may also be administered using an enteral and/or a parenteral administration route, including but not limited to oral, rectal, and/or intranasal delivery. In this manner, suitable parenteral administration routes may also include, but is not limited to intravascular administration, subcutaneous injection and/or deposition, direct application to the tissue of interest, and/or inhalation. As such, in some embodiments, suitable administration routes may include injection, infusion and/or direct injection into a target tissue.

In this manner, in an embodiment, at least one Alu RNA, transcribed in the forward direction and/or the reverse direction, of the miR-498 cistron and/or the nucleic acid molecule encoding the at least one Alu RNA, transcribed in the forward direction and/or the reverse direction, of the miR-498 cistron, may be administered to the subject either as naked RNA and/or DNA in combination with a delivery reagent, such that the at least one Alu RNA of the miR-498 cistron and/or the nucleic acid molecule encoding the at least one Alu RNA of the miR-498 cistron may be encoded by a recombinant plasmid and/or a viral vector. Recombinant plasmids and/or viral vectors may include sequences that express the at least one Alu RNA of the miR-498 cistron and/or Alu RNA portion thereof.

In an embodiment, the method may comprise the step of incorporating at least one liposome to deliver the at least one Alu RNA, transcribed in the forward direction and/or the reverse direction, of the miR-498 cistron to the subject. As such, in this embodiment, the at least one liposome may be formed from standard vesicle-forming lipids, such that neutral and/or negatively charged phospholipids and/or a sterol, such as cholesterol, may be included. The selection of lipids is generally guided by consideration of several factors, including but not limited to the desired liposome size and/or half-life of the liposomes in the blood stream. A variety of methods are known in the art for preparing liposomes (see, for example, Szoka et al., Ann. Rev. Biophys. Bioeng. 9:467, 1980).

Additionally, in an embodiment, the method may comprise the step of incorporating at least one polymer to deliver the at least one Alu RNA, transcribed in the forward direction and/or the reverse direction, of the miR-498 cistron to the subject. In this manner, the at least one polymer that may be used to deliver therapeutic nucleic acid molecules have been described (see, for example, Zhang et al., J Control Release. 123 (1): 1-10, 2007; Vorhies et al., Methods Mol. Biol. 480:11-29, 2009). Moreover, in some embodiments, at least one polypeptide carrier may also be used to administer the at least one Alu RNA of the miR-498 cistron to the subject (see, for example, Rahbek et al., J. Gene Med. 10:81-93, 2008).

In an embodiment, the nucleic acid molecules may be administered in any appropriate manner known in the art, such as with at least one pharmaceutically acceptable carrier. Pharmaceutically acceptable carriers may be determined in part by the particular composition being administered, as well as by the particular method used to administer the composition.

Accordingly, in this embodiment, a wide variety of suitable formulations of pharmaceutical compositions may be available to incorporate the at least one Alu RNA, transcribed in the forward direction and/or the reverse direction, of the miR-498 cistron.

In addition, in an embodiment, the method may comprise the step of preparing for parenteral administration. As such, the parental administration may include sterile aqueous and/or non-aqueous solutions, suspensions, and/or emulsions. In this same manner, in some embodiments, the method may comprise the step of incorporating intravenous vehicles to administer the miR-498 cistron and/or the Alu portion, transcribed in the forward direction and/or the reverse direction to the subject. The intravenous vehicles may include but are not limited to fluid and/or nutrient replenishers, electrolyte replenishers (such as those based on Ringer's dextrose), and the like.

As such, in an embodiment, the administration of the miR-498 cistron and/or the at least one Alu RNA, transcribed in the forward direction and/or the reverse direction, of the miR-498 cistron to the subject may be accomplished by single and/or multiple doses. In this manner, in this embodiment, the dose required may vary from subject to subject depending on the species, age, weight and/or general condition of the subject, the particular nucleic acid molecule being used and/or its mode of administration. In this manner, an appropriate dose may be determined by one of ordinary skill in the art using only routine experimentation.

The following examples are provided for the purpose of exemplification and are not intended to be limiting.

### EXAMPLES

### Example 1

### Materials for Mir-498 (46) Cistron Detection and Antiviral Response

This example describes the materials used for the studies described in Example 2 and Example 4.

### Human Studies

Deidentified human term placentas from normal pregnancies were obtained with written, informed consent under the University of South Florida IRB Protocol 00015578. Previously banked first trimester placental paraffin specimens obtained from voluntary terminations of uncomplicated pregnancies were used after approval by the University of South Florida Institutional Review Board (Protocol 00019472). Written informed consent was received from patients prior to inclusion in the study. Human left ventricle samples were obtained from viable, non-transplantable deidentified human hearts from three female (age 56, 65 and 85 years) and one male (age 52 years) brain-dead donors after consent was obtained from next of kin in accordance with Florida State Statutes and The Declaration of Helsinki. Hearts were donated through the Life Link Foundation. An MTA has been executed enabling transfer of non-transplantable organs for research at the University of South Florida.

### Animal Studies

C2MCA^{Δ/Δ} mice were generated and kindly provided by the RIKEN BRC through the National Bio-Resource Project of the MEXT/AMED, Japan. Breeding and experimental procedures were conducted with prior approval of the Animal Care and Use Committee at the University of South Florida under protocols 6183M and 9205R, respectively. WT (C57BL/6) and C2MCA^{Δ/Δ} mice were housed under standard conditions. Dams between approximately 4-7 months of age and 19-25 g weight were used for mating and the detection of the vaginal mucus plug was taken to be indicative of E0.5. WT and C2MCA^{Δ/Δ} male and female fetuses were used in the study. For in situ hybridization E18.5 WT placentas were used. For B1 RT-PCR and C2MC miRNA RT-qPCR E11.5 WT and C2MCA^{Δ/Δ} placentas were used, whereas for in vivo ZIKV experiments E14.5 WT and C2MC^{Δ/Δ} placentas and fetal heads were used.

### Cell Culture

AD-293 cells (Stratagene Cat #240085), 293T cells (ATCC CRL-3216), DICER-Ko derived from 293T cells (2-20 cells provided by Dr. Bryan Cullen, Duke University), DROSHA-Ko derived from 293T cells (provided by Dr. David A. Williams, Harvard Medical School), MAVS-Ko derived from 293T cells (provided by Dr. Young Bong Choi, Johns Hopkins University School of Medicine) and Vero-E6-high ACE2 (BEI Resources #NR-53726) cells were grown in DMEM (Genesee Scientific #25500) supplemented with 10% heat-inactivated fetal bovine serum (FBS; Millipore-Sigma #F4135). HTR8/SVneo (ATCC CRL-3271) cells were cultured in RPMI 1640 (Gibco #11875) supplemented with 10% heat-inactivated FBS. Vero cells (ATCC CCL81, a gift from Dr. Bala Chandran, University of South Florida) were grown in DMEM/F-12, HEPES, no phenol red (Gibco, #11039021) with 10% non-heat inactivated FBS (Corning #35-010-CV). HEp-2 cells (ATCC CCL-23; a gift from Dr. Subra Mohapatra, University of South Florida) and Hela cells (ATCC CCL-2) were grown in minimum essential medium (Gibco #11095080) and Eagle's minimum essential medium (ATCC 30-2003), respectively, supplemented with 10% heat-inactivated FBS. All cell culture media was supplemented with 1% Pen Strep (Gibco #15070063) and 1% L-glutamine (Gibco #25030081). Mouse trophoblast stem (mTS) cells were provided by the RIKEN BRC through the National BioResource Project of the MEXT, Japan and grown as previously described with minor modifications. Briefly, mTS cells were cultured in plates coated with 15 μg/mL human plasma fibronectin (EMD Millipore FC010) for 2 hours in CDM/FAXY media, generated by combining 1:1 Neurobasal medium (Gibco #21103049): DMEM/F-12 (Ham's) (Gibco #11320033) supplemented with 1% KnockOut serum (Gibco #10828028), 1% N-2 supplement (Gibco #17502048), 1% B-27 supplement (Gibco #17504044), 1% Pen Strep (Gibco #15070063), 1% GlutaMax (Gibco #35050061), 0.05% bovine serum albumin (EMD Millipore #126575), 150 μM 2-mercaptoethanol (Gibco #21985023), 50 ng/ml recombinant murine FGF-basic (PeproTech #450-33), 20 ng/mL recombinant human/murine/rat activin A (PeproTech #120-14E), 10 μM XAV939 (PeproTech #2848932) and 5 UM Y-27632 dihydrochloride (PeproTech #1293823). Medium was replaced every 2 days and to dissociate cells between passages TrypLE Express (Gibco #12604013) was used. 293T-ACE2 cells were generated by transducing 293T cells with pLenti-hACE2-hygro expressing lentivirus, a gift from Dr. Neville Sanjana (Addgene plasmid #161758; http://n2t.net/addgene: 161758; RRID: Addgene_161758), followed by selection with hygromycin (InvivoGen #anti-hg-1). All cell lines were cultured at 37° C. and 5% CO₂. Cell line sex determination and authentication were not performed. However, the cell genotypes were confirmed by RT-PCR, RT-qPCR and Western blotting and were all tested routinely with the Universal Mycoplasma Detection Kit (ATCC 30-1012K) to ensure that they were free of Mycoplasma contamination.

### Example 2

### Methods for Mir-498 (46) Cistron Detection and Antiviral Response in a Human Placenta

This example describes the experimental procedures used for the studies described in Example 4.

### Cell Transfections

For C19MC transcriptional activation cells are first transfected. Briefly, cells were transfected with a 1:1:1 mass ratio of dCAS9-VP64-GFP (Addgene plasmid #61422; http://n2t.net/addgene: 61422; RRID: Addgene_61422), MS2-P65-HSF1-Hygro (Addgene plasmid #61426; http://n2t.net/addgene: 61426; RRID: Addgene_61426) and the lenti sgRNA (MS2)_zeo backbone plasmid (Addgene plasmid #61427; http://n2t.net/addgene: 61427; RRID: Addgene_61427). C19MC specific sgRNAs #759, #620 and the CYP19A1 specific sgRNA #125.3 (Table 3, oligos #1-3) were cloned into the lenti sgRNA (MS2)_zeo backbone plasmid. AD-293, 293T, DICER-Ko, DROSHA-Ko, MAVS-Ko cells were transfected using Lipofectamine 2000 (Invitrogen #11-668-019) HTR8/SVneo cells using Lipofectamine 3000 (Invitrogen #3000015). The culture medium was replaced after 24 hours, and the transfected cells were incubated for a total of 60-72 hours.

For pattern recognition receptor knock down experiments cells were transfected with MAVS siRNA (h) (Santa Cruz Biotechnology, Inc; sc-75755), PKR siRNA (h) (Santa Cruz Biotechnology, Inc; sc-36263), or Control siRNA-A (Santa Cruz Biotechnology, Inc; sc-37007). For TLR3 (i.e., human) Silencer Select Validated siRNA (Life Technologies Corporation, s235; Cat #4427038), Silencer Select Negative Control siRNA #2 (Life Technologies Corporation; Cat #4390846) was used. After 24 hours, cells were transfected with 759-SAM or BB-SAM for 48 hours.

For experiments that studied the autocrine/paracrine effects of IFNL3, cells were transfected with 759-SAM, BB-SAM or GFP. After six hours, the media was replaced with or without 400 ng/ml of anti-human IFNL3 antibody (R&D Systems Cat #DY1598B-05) and collected after 66 hours.

Next, mTS cells were transfected with miR-467b-5p (Life Technologies #MC11605), miR-466b/c/p-3p (Life Technologies #MC19359) mimic, miRNA mimic negative control (ThermoFisher Scientific #4464058), IVT BI SINE RNA or control GFP-mRNA using Lipofectamine 3000 (Invitrogen #3000015), according to the manufacturer's instructions for 24 hours.

AD-293 and mTS cells were then transfected with 10 μg/mL of Poly(I:C) HMW (InvivoGen #31852-29-6) for 24 hours and collected for RNA extraction to be used as positive controls in RT-PCR and RT-qPCR experiments.

### In Vitro Transcription

To generate Alu RNA transcripts, the bacterial artificial chromosome containing C19MC (BACPAC Resources, RP11-1055017) was used as a template with the previously described 5′-exon (2) and 3′-exone (2) primers (TABLE 3, SEQ ID NO: 4) to obtain a PCR product containing fragments of C19MC. PCR products were cloned into the pCR™ 4Blunt-TOPO™ vector by one-step cloning using the Zero Blunt™ TOPO™ PCR Cloning Kit (Invitrogen #45-0031). Following transformation into GC competent cells (Genesee Scientific #42-661), single colonies were picked and sequenced. A colony that contained a fragment of C19MC that aligned to chr19: 53,678,369-53,681,590 on the GRCh38/hg38 human genome was used as the template for PCR amplification. For IVT AluJb, AluSx, and AluSz in the forward direction, we included the T7 promoter sequence in the forward primer (TABLE 3, SEQ ID NO: 5-7), and to IVT the Alus in the reverse strand, we added the T7 promoter sequence to the reverse primer (Table 3, SEQ ID NO: 8-10).

To generate C2MC B1 RNA in the forward direction, C2MC B1 consensus sequence was PCR amplified using forward and reverse primers (TABLE 3, SEQ ID NO: 11) and cDNA of WT mTS cells that was generated using a mixture of random primer hexamers and anchored-dT primer (New England Biolabs #S1330S) and M-MuLV reverse transcriptase (New England Biolabs #M0253L). The B1 PCR product was cloned into the pCR™ 4Blunt-TOPOT vector by one-step cloning using the Zero Blunt™ TOPOT PCR Cloning Kit (Invitrogen Cat #45-0031). Following transformation, single colonies were picked and sequenced. A colony that contained the C2MC B1 in the forward direction was used as the template for PCR amplification using the same primers (TABLE 3, SEQ ID NO: 11) but the forward primer contained T7 promoter (TABLE 3, SEQ ID NO: 12). The resulting PCR product was used as a template for IVT.

Control GFP mRNA was IVT as previously described. Briefly, human 8-globin 3′ UTR (132 bp) was amplified using HeLa genomic DNA, extracted using the Monarch Genomic DNA Purification Kit (New England Biolabs #T3010S) as a template, with forward and reverse primer that include EcoRI restriction site (TABLE 3, SEQ ID NO: 13). PCR product was EcoRI digested and cloned into the pLL3.7 plasmid a gift from Luk Parijs (Addgene plasmid #11795; http://n2t.net/addgene: 11795; RRID: Addgene_11795). Template for GFP IVT were generated by PCR using forward primer containing a T7 promoter, 5′ UTR of human β-globin and the first 26 bases of GFP and the same reverse primer of the human β-globin 3′ UTR (TABLE 3, SEQ ID NO: 14).

The HiScribe T7 High Yield RNA synthesis kit (New England Biolabs, E2040S) was used for IVT according to the manufacturer's instructions with 100% substitution of uridine with pseudouridine (TriLink Biotechnologies, N-1019) to reduce the immunogenicity of the IVT mRNA. After ammonium acetate precipitation, IVT RNA was washed with 70% ethanol, resuspended in H₂O, quantified by spectrophotometry, and stored at −80° C. until use.

The GFP RNA was then capped using the ScriptCap m7G Capping System (CellScript, C-SCCE0625) and poly-A tailing using the A-Plus Poly(A) Polymerase Tailing Kit (CellScript, C-PAP5104H) prior to ammonium acetate precipitation.

### HeLa Heat Shock

Heat-shock-induced Alu expression was then performed. As such, Hela cells were briefly heat-shocked at 45° C. for 30 minutes and then allowed to recover for 4 hours in standard culture conditions at 37° C. before total RNA extraction.

### Protein Extractions and Western Blotting

Cells were lysed in ice-cold lysis buffer containing 40 mM HEPES [pH 7.5], 120 mM NaCl, 1 mM EDTA, 10 mM pyrophosphate, 10 mM glycerophosphate, 50 mM NaF, 0.5 mM orthovanadate, EDTA-free protease inhibitors [Roche] and 0.3% CHAPS for 20 minutes. After clearing the lysates by centrifugation at 13,000×g for 10 min at 4° C., samples were stored at −80° C.

Protein lysates were fractionated by SDS-PAGE and transferred to BioTrace NT Nitrocellulose transfer membranes (Pall Life Sciences Cat #27376-991). Membranes were incubated overnight at 4° C. with rabbit anti alpha tubulin (Cell Signaling Technology Cat #2144, RRID: AB_2210548, 1:1000), rabbit anti-DICER1 (D38E7) (Cell Signaling Technology Cat #5362S, RRID: AB_10692484, 1:1000), rabbit anti-DROSHA (D28B1) (Cell Signaling Technology Cat #3364, RRID: AB_2238644, 1:1000), rabbit anti-IRF7 (Cell Signaling Technology Cat #4920, RRID: AB_2127551, 1:1000), rabbit anti-human MAVS (Cell Signaling Technology Cat #3993; RRID: AB_823565, 1:1000), rabbit anti-human PKR (Cell Signaling Technology Cat #3072S; RRID: AB_2277600, 1:1000) or rabbit anti-GAPDH (14C10) (Cell Signaling Technology Cat #2118, RRID: AB_561053, 1:2000) primary antibodies in Odyssey Blocking Buffer (PBS, LI-COR Biosciences, Fisher scientific 15590545). After washing three times in TBS-Tween, the membranes were incubated for 20-minutes at room temperature with IRDye 680 donkey anti-rabbit IgG secondary antibody (LI-COR Biosciences Cat #926-68073, RRID: AB_10954442, 0.2 μg/ml). Membranes were washed three times in TBS-Tween, imaged on Odyssey CLX-2050 imaging system (LI-COR instrument) and analyzed using Image Studio acquisition software (LI-COR, ver 5.2).

### RNA Extraction, RT-PCR and RT-qPCR

Cultured cells, term human placenta, mouse placentas, fetal heads and spleens were lysed using Qiazol lysis reagent (Qiagen) on ice. Total RNA was extracted using the miRNeasy Kit (Qiagen #217004) and treated with RNase-free DNase (Qiagen #79254) according to the manufacturer's instructions. RNA was quantified spectrophotometrically at 260 nm and stored at −80° C.

One μg total RNA was used for reverse transcription with a mixture of random primer hexamers and anchored-dT primer (New England Biolabs #S1330S) or oligo (dT) primers (New England Biolabs #S1316S) and M-MuLV reverse transcriptase (New England Biolabs #M0253L) according to the manufacturer's recommendations. For miRNA, 0.5 μg total RNA was reverse transcribed using the TaqMan miRNA Reverse Transcription Kit (Thermo Fisher Scientific #4366596) according to the manufacturer's instructions.

To assess relative mRNA and miRNA expression levels Applied Biosystems™ TaqMan™ Fast Universal PCR Master Mix (2×), no AmpErase™ UNG (Fisher Scientific #4352042) and the TaqMan RT-qPCR probes were used according to the manufacturer's instructions and run on QuantStudio 3 Real-Time PCR system (TABLE 2, provided below). The following TaqMan RT-qPCR probes were used: IFNL2/3 (Hs04193048_gH), IFNB1 (Hs01077958_s1), ISG15 (Hs00192713_m1), OAS1 (Hs00973637_m1), APOBEC2 (Hs00199012_m1), APOBEC3G (Hs00222415_m1), TNF (Hs01113624_g1), IL6 (Hs00174131_m1), IFITM1 (Hs01652522_g1), TLR3 (Hs00152933_m1), EIF2AK2 (Hs00169345_m1), CYP19A1 (Hs00903411_m1), IFNA2 primers (forward 5′-CTTGAAGGACAGACATGACTTTGGA, Reverse 5′-GGATGGTTTCAGCCTTTTGGA and FAM probe 5′-TTCCCCAGGAGGAGTTTGGCAACC), GAPDH (Hs02786624_g1), hsa-miR-517a (002402), hsa-miR-515-5p (001112), hsa-miR-516b (001150), hsa-miR-518c (002401), hsa-miR-519d (002403), U18 (001204), mouse Ifnl3 (Mm00663660_g1), mouse Ifna2 (Mm00833961_s1), mouse Ifnb1 (Mm00439552_s1) mmu-miR-467a (002587), mmu-miR-467* (001671), mmu-miR-466b/c/p (464896_mat), and mmu-snoRNA202 (001232). For VSV, ZIKV, RSV and SARS-CoV-2, GAPDH, Polr2a RT-qPCR were performed using PowerUpSYBR Green Master Mix (Applied Biosystems #A25741) using the primer sets described in TABLE 1, SEQ ID NO: 15-20. For MAVS RT-qPCR the predesigned KiCqStart SYBR Green primers for human MAVS H_MAVS_2 (Millipore Sigma). Samples were run in triplicate and the relative expression were then calculated.

RT-PCR for IFNL2/3, Alu, GAPDH, and Polr2a were performed using primers listed in TABLE 1, SEQ ID NO: 19-22, RT-PCR for multiple subtypes of human IFNA and IFNB were performed with the previously described primers found within TABLE 1, and B1 RT-PCR was performed using SEQ ID NO: 11 with either Taq DNA Polymerase with ThermoPol buffer (New England Biolabs #M0267X) or PowerUpSYBR Green Master Mix (Applied Biosystems #A25741). The PCR products were run on a 1.5% agarose gel. For fl-Alu:sc-Alu ratio quantifications, the band intensities of the PCR products were quantified with ImageJ software (version 1.53u) and normalized to GAPDH after subtracting background intensity. To control for genomic DNA contamination, the Alu PCR reaction was also run on the total RNA of the same samples and visualized on an agarose gel. The Alu PCR products were gel purified and cloned into the pCR™ 4-TOPOR TA vector and pCRT2.1-TOPOR vector using the TOPO™ TA Cloning™ Kit (Invitrogen Cat #45-0030). After transformation, plasmid DNA was extracted from single colonies and sent for sequencing using T3 and T7 primers or M13 forward and reverse primers, as shown in TABLE 3. The sequencing results are shown in TABLE 3, provided below, after trimming the vector sequences using the flanking EcoRI restriction sites. The sequences were verified to be Alu SINEs using BLAT alignment against the human GRCh38/hg38 genome in the USCS Genome Browser.

Dot Blotting of dsRNA

One μg of total RNA in 10 μL were loaded onto a wet Biotrace Nitrocellulose membrane (Pall Biosciences) using a Minifold dot-blotter (Schleicher & Schuell, Inc.). The membrane was then baked at 80° C. for 1 hour before blocking for 1 hour at room temperature in Odyssey Blocking Buffer (PBS, LI-COR Biosciences, Fisher scientific 15590545) and probed overnight at 4° C. with J2 mouse monoclonal anti-dsRNA antibody (Scicons, 1:1000). After TBS-Tween washing the membranes were incubated with IRDye 800CW goat anti-mouse secondary antibody (LI-COR Biosciences Cat #926-32210, RRID: AB_621842, 0.2 μg/mL) at room temperature for 20 minutes. The resulting immunoblots were washed again with TBS-Tween, scanned and quantitatively assessed using the Odyssey CLX-2050 imaging system (LI-COR instrument) and analyzed using Image Studio acquisition software (LI-COR, ver 5.2).

### ELISA

Enzyme-linked immunosorbent assays for human IFNL1/3 (R&D Systems #DY1598B-05) and all subtypes of IFNA (R&D Systems #DFNASO) were performed according to the manufacturer's instructions on conditioned cell culture media collected 72 hours after transfection with BB-SAM or 759-SAM.

### In Situ Hybridization and Immunostaining

Paraffin-embedded term human placental sections were deparaffinized in xylene and rehydrated by a series of graded alcohol washes. Control term placental sections were subjected to treatment with RNase A (100 mg/mL, Sigma-Aldrich, R6148) or DNase I (2000 units/mL, New England Biolabs, M0303S) at 37° C. for 30 minutes. OCT-embedded mouse placental sections were dried at 55° C. overnight, rehydrated with 2 PBS washes and treated with proteinase K (1.5 mg/mL) for 10 minutes. In situ hybridization was performed as previously described using 40 nm 5′,3′ digoxigenin-labeled locked nucleic acid probe for the C19MC hsa-miR-517a/b (Exiqon, 611715-360), positive strand Alu (Qiagen, 339500 LCD0162058-BKG), the C2MC mmu-miR-669a-3p (Qiagen, 339111 YD00616024-BCG), B1 (Qiagen, 339115 YCD0077239-BCG) or scrambled (negative) control (Exiqon, 90005). Hybridization and post-hybridization graded SSC washes were performed at 55° C. The sections were then blocked, and the probes were detected using alkaline phosphatase conjugated sheep anti-digoxigenin Fab fragments (Roche, 11093274910). The signal was developed using NBT/BCIP (Roche, 11697471001) as a substrate, which produces a dark-blue/indigo precipitating dye, followed by nuclear counterstaining with Nuclear Fast Red (Vector Laboratories, H-3403). The sections were dried and covered with mounting medium for image analysis.

Cytokeratin and vimentin immunostaining were performed as previously described using mouse-anti-cytokeratin 7 (Dako M7018, 1:600, RRID: AB_2134589) and chicken-anti-vimentin (Abcam ab39376, RRID: AB_778827, 1 ug/mL) primary antibodies. Biotinylated horse-anti-mouse (Vector Laboratories BA-2000, 3.75 ug/ml, RRID: AB_2313581) secondary antibody with avidin-biotin-peroxidase complex (Vectastain ABC Kit, pk6200, Vector Laboratories) was used to detect cytokeratin and the signal was developed using 3,3-diaminobenzidine (sk-4100, Vector Laboratories) as a substrate. To detect vimentin, the donkey-anti-chicken (Jackson ImmunoResearch 703-065-155, 1.2 ug/ml, RRID: AB_2313596) secondary antibody was used with avidin-biotin-alkaline phosphatase (Vectastain ABC-AP, Vector laboratories AK-5200) was used. The signal was developed using Vector Red AP substrate (Vector Red, Vector Laboratories SK-5100) followed by nuclear counterstaining with hematoxylin and mounting for later image analysis.

Next, Immunofluorescent staining was then performed. Briefly, term human placental sections were permeabilized with 0.1% TritonX100 at room temperature for 30 minutes and blocked with 3% (w/v) bovine serum albumin for 30 min at room temperature. The sections were then probed with anti-dsRNA mouse monoclonal J2 antibody (Scicons, 1:50) at 4° C. overnight. After washing with PBS, the sections were probed with donkey anti-mouse IgG (H+L) highly cross-adsorbed secondary antibody, Alexa Fluor™ 647 (Invitrogen Cat #A-31571, RRID: AB_162542, 2 μg/mL) at room temperature for 30 minutes. Sections were then mounted with Prolong Diamond Antifade Mountant with DAPI (Thermo Fisher Scientific Cat #P36971) nuclei counterstaining and visualized with a FluoView FV1200 inverted laser scanning confocal microscope (Olympus).

### Virus Propagation and Titration

Handling of ZIKV, RSV, and VSV were done according to biosafety level (BSL)-2 guidelines and SARS-CoV-2 according to BSL-3 guidelines. VSV Indiana strain, ZIKV PRVABC59 (Puerto Rico strain), and RSV A2 strain were kindly provided by the University of South Florida. SARS-CoV-2 (Isolate New York 1-PV08001/2020) was obtained from BEI Resources (#NR-52368). VSV and ZIKV were propagated in Vero cells, RSV in Hep-2 cells, and SARS-CoV-2 in Vero-E6-ACE2 cells using modified protocols developed from previously described methods. Briefly, cells were seeded into T-75 or T-175 flasks and allowed to grow until 90% confluency. Cells infected with the respective virus at a multiplicity of infection (MOI) of 0.01-0.1 in low-serum infection media and incubated at 37° C./5% CO₂ for 1-2 hours, gently rocking the flasks every 10-15 minutes. At the end of this period, cell culture media was added to the flasks and further incubated for several days. When most cells showed cytopathic effects, cells and supernatant were collected, clarified by centrifugation, and stored at −80° C. until used. RSV and ZIKV for in vivo studies were further purified by ultracentrifugation as previously described.

The harvested virus stocks were then titrated by plaque assays. Briefly, serial 10-fold dilutions of each virus stock were used in duplicate to infect confluent 6-well plates of Vero, Hep-2, or Vero-E6-ACE2 cells for the respective virus as mentioned above in low-serum infection media. After gently rocking the plates every 10-15 minutes while incubating at 37° C./5% CO₂ for 1-2 hours, the infection media was removed and 1% agarose overlay media was added. Infected cells were incubated for several days until plaques appeared. The cells were then fixed (with 40% methanol for VSV and ZIKV and 1% formaldehyde for RSV) and stained with dyes (0.4% Crystal violet for VSV and ZIKV and 0.05% neutral red for RSV). Plaques were counted in duplicate per dilution series, and the titer of the virus stock was calculated as plaque-forming units (PFU)/mL. For SARS-CoV-2 titration, two layers of solid-overlay media consisting of 2% noble agar were added two days apart, with the second layer containing 0.33% neutral red and directly visualized using a white light transilluminator. Plaques were counted, and titer was calculated as PFU/mL.

### In Vitro and In Vivo Viral Infections

After 60 or 72 hour transfection of cells with BB-SAM or 759-SAM, the supernatant from each well was removed and saved at 37° C. while the cells were infected with VSV (MOI=0.2), ZIKV (MOI=1), RSV (MOI=0.5) or SARS-CoV-2 (MOI=0.01) in serum-free media. After 90 minutes, the infection media was replaced with the saved supernatant of each respective well. The cells were collected for RNA extraction after 8 hours infection with VSV or 24 hours with Zika, RSV or SARS-CoV-2.

WT and C2MCA^{Δ/Δ} mTS cells or mTS cells transfected with IVT control GFP-mRNA, IVT B1-forward RNA, miR-467b-5p- (Life Technologies #MC11605), miR-466b/c/p-3p- (Life Technologies #MC19359) or control-(ThermoFisher Scientific #4464058) mimic for 4 hours were infected with ZIKV (MOI=0.1) in basal media. After 90 minutes, the media was replaced with the supernatant preserved from each well and cells were collected for RNA extraction 24 hours after infection.

For experiments exploring the effect of type I IFN receptor inhibition on the response of mTS cells to ZIKV, WT and C2MCA^{Δ/Δ} mTS cells were treated with 200 μg/mL anti-mouse IFNAR1 mAb (clone MAR1-5A3, Leinco Technologies Cat #1-401, RRID: AB_2491621) for 2 hours and then infected with ZIKV (MOI=0.1). After 90 minutes, the infection media was removed and the supernatant saved from each respective well was added back with or without 1 ng/ml of recombinant mouse IFNA2 protein (carrier free, R&D Systems Cat #10149-IF-010) and cells were collected for RNA extraction 24 hours after infection.

For the in vivo ZIKV infections, E8.5 pregnant dams were intraperitoneally (IP) injected with 2.5 mg/mouse anti-IFNAR1 mAb (clone MAR1-5A3, Leinco Technologies Cat #1-401, RRID: AB_2491621). The following day, mice were IP injected with purified ZIKV 104 pfu in 100 μL of sterile 1×PBS. After five days (on E14.5), the dams were sacrificed, and placentas, fetal heads, and the maternal spleens were harvested for total RNA extraction and ZIKV RT-qPCR. Fetal genotypes were determined using the previously described primers and confirmed by assessing the expression of C2MC-specific miR-467a in the placentas.

### Small RNA Sequencing and Data Analysis

Briefly, total RNA was extracted from triplicate cultures of AD-293 cells transfected with GFP, 759-SAM or 620-SAM and 293T and DICER1-Ko cells transfected with either BB-SAM or 759-SAM and grown for 72 hours, using the miRNeasy Kit (Qiagen, Cat #217004) and treated with RNase-free DNase (Qiagen, Cat #79254) according to the manufacturer's instructions.

For AD-293 cells, 2 μg of total RNA were converted into a small RNA (sRNA) cDNA library. The RNA input for each sample was ligated to a 3′ adaptor barcoded sequence, pooled, size selected, and gel purified, followed by 5′ adapter ligation and then subjected to size selection and gel purification. SuperScript III was used for second strand synthesis and the cDNA library preparation was completed with alkaline RNA hydrolysis and PCR amplification for 10 cycles.

For 293T and DICER-Ko cells, the RNA were organized in one batch of 12 samples, each containing 1.0 μg total RNA. For cDNA library preparation, 2.5 moles of Calibrator Set2 (standard calibrator), a set of ten 21-nt 5′-phosphorylated RNA oligos, was added. Each RNA sample was individually 3′-adapter-ligated. Up to 24 reaction products were pooled, 5′ adapter-ligated, PCR-amplified and sequenced in a single NextSeq500 lane. Reads were demultiplexed, mapped against a curated hg19-based miRNA reference-transcriptome, sorted, and tabulated into different RNA categories. Sequencing data were processed (Illumina software suite), followed by read extraction using an in-house RNA Sequencing Data Analysis Pipeline (RSDAP) specifying a size range of 16 to 45 nt and default parameters. Demultiplexed RNA sequencing data was mapped against our curated human reference transcriptome to obtain miRNA raw read and read frequency profiles and abundance of fragments of other RNA classes, such as tRNAs, snRNAs, scRNAs, and rRNAs. Mapped data were used to generate RNA summary tables, as well as detailed miRNA raw read and read frequency Tables that were used for differential expression analysis and unsupervised clustering, respectively.

Reads annotated as calibrator, expression system (plasmid & E. coli) marker and adapter were considered as reads of technical origin; those remaining were considered as reads derived from the sample. For differential expression analyses, tabulated shared raw reads of merged miRNAs reported by RSDAP were used to perform differential expression analyses using DESeq2, considering only miRNAs with at least five counts across all samples. Selected metadata categories (GFP, 759-SAM, 620-SAM and BB-SAM, 759-SAM), were used as experimental design parameters, comparing miRNA abundance differences between two distinctive sample groups. For all merged miRNAs, we considered a differential change in abundance as detected if the underlying base mean was at least 5 normalized counts and as statistically significant if the reported adjusted p-value was less than 0.05.

### RNA Sequencing and Bioinformatics Analysis

mRNA libraries of AD-293 cells were prepared by utilizing the Illumina TruSeq Stranded mRNA LT protocol using 500 ng total RNA and NEB's Protoscript II reverse transcriptase for the first-strand cDNA synthesis according to the manufacturer's protocol. Individual RNAseq libraries were quality controlled on an Agilent TapeStation with a High Sensitivity D1000 ScreenTape. Indexed samples were quantified using the Qubit dsDNA HS assay and were pooled at equimolar concentration (10 nM). The libraries were sequenced on an Illumina NextSeq. 500 sequencer 75-bp paired-end in mid-output mode in the Genomics Core Facility of The Rockefeller University.

From 293T and DICER-Ko cells, 0.1 μg total RNA was used for stranded total RNA library preparations (Illumina TruSeq, Cat #_20020596), and this workflow included a Ribo-Zero Human/Mouse/Rat RNA depletion step. Libraries were prepared with unique barcodes and pooled at equal molar ratios. The pool was denatured and sequenced on Illumina NextSeq 500 sequencer using high output V2 reagents and NextSeq Control Software v1.4 to generate 75 bp paired-end reads, following manufacture's protocol (Cat #15048776 Rev.E). mRNA sequencing reads were aligned to the human genome (GRChg38) using the RNASTAR aligner allowing for two mismatches. Read counts were generated using feature Counts, and differential expression analysis was completed using edgeR. Differentially expressed genes were considered significant with an FDR<0.1 and fold-change >2.0 up or down.

Gene set enrichment analysis was performed using the UC San Diego and Broad Institute GSEA software and Molecular Signatures Database (MSigDB).

TABLE 1

| | 5′-Forward Primer | 5′-Reverse Primer |
|---|---|---|
| GAPDH | CTGACTTCAACAGCGACACC | TAGCCAAATTCGTTGTCATACC |
| Alu | CCGGGTGCGGTGGCACACGCT | GCAATCTCCTTCTCACGGGTT |

TABLE 2

1. 50° C. for 2 minutes
2. 95° C. for 2 minutes
3. 95° C. for 1 second
4. 60° C. for 30 seconds
{Repeat Steps 3-4 36x}
5. 95° C. for 1 second
6. 60° C. for 20 seconds
7. 95° C. for 1 second
{Steps 5-7 Melt-curve phase}

TABLE 3

| | | | | hg38/ |
|---|---|---|---|---|
| Clone | Sequencing with M13 | Sequencing with M13 | | Human |
| number | forward primer | reverse primer | Span | Alu |
| HeLa | AATTCGCCCTTGCAATCTCC | GAATTCGCCCTTCCGGGTGC | 220 | AluSc8 |
| heat | TTCTCACGGGTTCAGGCAA | GGTGGCACACGCTTGTAGTC | | |
| shock_1 | TTCTCCTGCCTCAGCCTCC | CCAGCACTTTGGGAGGCCAA | | |
| | AAGTACCTGGGACTACAGG | GGCGGGCAGATCATGAGGTC | | |
| | CGCTGGCCACCATGCCCAG | AGGAAATTGAGACCAGCCTG | | |
| | CTAATTTTCTGTATTTTTAGT | GCTAACATGGTGAAACTCCGT | | |
| | AGAGACGGAGTTTCACCAT | CTCTACTAAAAATACAGAAAA | | |
| | GTTAGCCAGGCTGGTCTCA | TTAGCTGGGCATGGTGGCCA | | |
| | ATTTCCTGACCTCATGATCT | GCGCCTGTAGTCCCAGGTAC | | |
| | GCCCGCCTTGGCCTCCCAA | TTGGAGGCTGAGGCAGGAGA | | |
| | AGTGCTGGGACTACAAGCG | ATTGCCTGAACCCGTGAGAA | | |
| | TGTGCCACCGCACCCGGAA | GGAGATTGCAAGGGCGAATT | | |
| | GGGCGAATTC (SEQ ID NO: | C (SEQ ID NO: 2) | | |
| | 1) | | | |
| HeLa | AATTCGCCCTTGCAATCTCC | GAATTCGCCCTTCCGGGTGC | 220 | AluSc8 |
| heat | TTCTCACGGGTTCAAGCAAT | GGTGGCTCACGCTTGTAATCC | | |
| shock_2 | TCTCCTGCCTCAGCCTCCA | CAGCACTTTGGGAGGCCAAG | | |
| | AGTACCTGGGACTACAGGC | GCGGGCAGATCATGAGGTCA | | |
| | GCTGGCCACCATGCCCAGC | GGAAATTGAGACCAGCCTGG | | |
| | TAATTTTCTGTATTTTTGGTA | CTAACATGGTGAAACTCTGTC | | |
| | GAGACAGAGTTTCACCATG | TCTACCAAAAATACAGAAAAT | | |
| | TTAGCCAGGCTGGTCTCAA | TAGCTGGGCATGGTGGCCAG | | |
| | TTTCCTGACCTCATGATCTG | CGCCTGTAGTCCCAGGTACTT | | |
| | CCCGCCTTGGCCTCCCAAA | GGAGGCTGAGGCAGGAGAAT | | |
| | GTGCTGGGATTACAAGCGT | TGCTTGAACCCGTGAGAAGG | | |
| | GTGCCACCGCACCCGGAAG | AGATTGCAAGGGCGAATTC | | |
| | GGCGAATTCC (SEQ ID NO: | (SEQ ID NO: 4) | | |
| | 3) | | | |
| HeLa | GAATTCGCCCTTCCGGGTG | GAATTCGCCCTTGCAATCTCC | 220 | AluSc8 |
| heat | CGGTGGCACACGCTTGTAA | TTCTCACGGGTTCAAGCAATT | | |
| shock_3 | TCCCAGCACTTTGGGAGGC | CTCCTGCCTCAGCCTCCAAGT | | |
| | CAAGGCGGGCAGATCATGA | ACCTGGGACTACAGGCGCTG | | |
| | GGTCAGGAAATTGAGACCA | GCCACCATGCCCAGCTAATTT | | |
| | GCCTGGCTAACATGGTGAA | TCTGTATTTTTGGTGGAGACA | | |
| | ACTCTGTCTCCACCAAAAAT | GAGTTTCACCATGTTAGCCAG | | |
| | ACAGAAAATTAGCTGGGCA | GCTGGTCTCAATTTCCTGACC | | |
| | TGGTGGCCAGCGCCTGTAG | TCATGATCTGCCCGCCTTGGC | | |
| | TCCCAGGTACTTGGAGGCT | CTCCCAAAGTGCTGGGATTAC | | |
| | GAGGCAGGAGAATTGCTTG | AAGCGTGTGCCACCGCACCC | | |
| | AACCCGTGAGAAGGAGATT | GGAAGGGCGAATTC (SEQ ID | | |
| | GCAAGGGCGAATTC (SEQ | NO: 6) | | |
| | ID NO: 5) | | | |
| HeLa | GAATTCGCCCTTGCAATCTC | GAATTCGCCCTTCCGGGTGC | 203 | AluSc |
| heat | CTTCTCACGGGTTCAAGCG | GGTGGCACACGCTTGTAATC | | |
| shock_4 | ATTCTCCTGCCTCAGCCTC | CCAGCACTTTGGGAGGTCGA | | |
| | CCAACTAACTGGGACTACA | GGCGGGCGGATCACGAGGTC | | |
| | AGCGCGCGCCACCACGCC | AGGAGATCGAGACCATCCTG | | |
| | CAGCTAATTTTTGTATTTTTA | GCCAACATGGTGAAGCCCCG | | |
| | GTAGAGACGGGGCTTCACC | TCTCTACTAAAAATACAAAAAT | | |
| | ATGTTGGCCAGGATGGTCT | TAGCTGGGCGTGGTGGCGCG | | |
| | CGATCTCCTGACCTCGTGA | CGCTTGTAGTCCCAGTTAGTT | | |
| | TCCGCCCGCCTCGACCTCC | GGGAGGCTGAGGCAGGAGAA | | |
| | CAAAGTGCTGGGATTACAA | TCGCTTGAACCCGTGAGAAG | | |
| | GCGTGTGCCACCGCACCCG | GAGATTGCAAGGGCGAATTC | | |
| | GAAGGGCGAATTC (SEQ ID | (SEQ ID NO: 8) | | |
| | NO: 7) | | | |
| HeLa | GAATTCGCCCTTCCGGTGC | GAATTCGCCCTTGCAATCTCC | 198 | AluSc |
| heat | GGTGGCACACGCTTGTAAT | TTCTCACGGGTTCAAGCGATT | | |
| shock_5 | CCCAGCACTTTGGGAGGTC | CTCCTGCCTCAGCCTCCCAAC | | |
| | GAGGCGGGCGGATCACGA | TAACTGGGACTACAAGCGCG | | |
| | GGTCAGGAGATCGAGACCA | CGCCACCACGCCCAGCTAAT | | |
| | TCCTGGCCAACATGGTGAA | TTTTGTATTTTTAGTAGAGACG | | |
| | GCCCCGTCTCTACTAAAAAT | GGGCTTCACCATGTTGGCCA | | |
| | ACAAAAATTAGCTGGGCGT | GGATGGTCTCGATCTCCTGAC | | |
| | GGTGGCGCGCGCTTGTAGT | CTCGTGATCCGCCCGCCTCG | | |
| | CCCAGTTAGTTGGGAGGCT | ACCTCCCAAAGTGCTGGGATT | | |
| | GAGGCAGGAGAATCGCTTG | ACAAGCGTGTGCCACCGCAC | | |
| | AACCCGTGAGAAGGAGATT | CGGAAGGGCGAATTC (SEQ ID | | |
| | GCAAGGGCGAATTC (SEQ | NO: 10) | | |
| | ID NO: 9) | | | |
| HeLa | GAATTCGCCCTTGCAATCTC | GAATTCGCCCTTCCGGGTGC | 220 | AluSx3 |
| heat | CTTCTCACGGGTTCAAGCA | GGTGGCACACGCTTGTAATC | | |
| shock_6 | ATTCTCCTGCCTCAGCCTC | CCAGCACTTTGGGAGGCCAA | | |
| | CAAGTACCTGGGACTACAG | GGCGGGCAGATCATGAGGTC | | |
| | GCGCTGGCCACCATGCCCA | AGGAAATTGAGACCAGCCTG | | |
| | GCTAATTTTCTGTATTTTTA | GCTAACATGGTGAAACTCTGT | | |
| | GTAGAGACAGAGTTTCACC | CTCTACTAAAAATACAGAAAA | | |
| | ATGTTAGCCAGGCTGGTCT | TTAGCTGGGCATGGTGGCCA | | |
| | CAATTTCCTGACCTCATGAT | GCGCCTGTAGTCCCAGGTAC | | |
| | CTGCCCGCCTTGGCCTCCC | TTGGAGGCTGAGGCAGGAGA | | |
| | AAAGTGCTGGGATTACAAG | ATTGCTTGAACCCGTGAGAAG | | |
| | CGTGTGCCACCGCACCCGG | GAGATTGCAAGGGCGAATTC | | |
| | AAGGGCGAATTC (SEQ ID | (SEQ ID NO: 12) | | |
| | NO: 11) | | | |
| HeLa | GAATTCGCCCTTGCAATCTC | GAATTCGCCCTTCCGGGTGC | 222 | AluSx1 |
| heat | CTTCTCACGGGTTCAAGCT | GGTGGCTTCACGCTTGTAATC | | |
| shock_7 | ATTCTCCTGCCTCAGCCTC | TCAGCACTTTGGGAGGCTGA | | |
| | CCGAGTAGCTGGGACTACA | GGCGGGCGGATCACCTGAGG | | |
| | GGTGCCCACCACCATGCCC | TCAGGAGTTTGAGACCAACCT | | |
| | AGCTACGTTTTGTATTTTTA | GGCCAACACAATGAAACCCC | | |
| | GTAGAGATGGGGTTTCATT | ATCTCTACTAAAAATACAAAAC | | |
| | GTGTTGGCCAGGTTGGTCT | GTAGCTGGGCATGGTGGTGG | | |
| | CAAACTCCTGACCTCAGGT | GCACCTGTAGTCCCAGCTACT | | |
| | GATCCGCCCGCCTCAGCCT | CGGGAGGCTGAGGCAGGAGA | | |
| | CCCAAAGTGCTGAGATTAC | ATAGCTTGAACCCGTGAGAAG | | |
| | AAGCGTGTGCCACCGCACC | GAGATTGCAAGGGCGAATTC | | |
| | CGGAAGGGCGAATTC (SEQ | (SEQ ID NO: 14) | | |
| | ID NO: 13) | | | |
| HeLa | GAATTCGCCCTTGCAATCTC | GAATTCGCCCTTCCGGGTGC | 220 | AluSx3 |
| heat | CTTCTCACGGGTTCAAGCA | GGTGGCACACGCTTGTAATC | | |
| shock_8 | ATTCTCCTGCCTCAGCCTC | CCAGCACTTTGGGAGGCCAA | | |
| | CAAGTACCTGGGACTACAG | GGCGGGCAGATCATGAGGCC | | |
| | GCGCTGGCCACCATGCCCA | AGGAAATTGAGACCAGCCTG | | |
| | GCTAATTTTCTGTATTTTTA | GCTAACATGGTGAAACTCTGT | | |
| | GTAGAGACAGAGTTTCACC | CTCTACTAAAAATACAGAAAA | | |
| | ATGTTAGCCAGGCTGGTCT | TTAGCTGGGCATGGTGGCCA | | |
| | CAATTTCCTGGCCTCATGAT | GCGCCTGTAGTCCCAGGTAC | | |
| | CTGCCCGCCTTGGCCTCCC | TTGGAGGCTGAGGCAGGAGA | | |
| | AAAGTGCTGGGATTACAAG | ATTGCTTGAACCCGTGAGAAG | | |
| | CGTGTGCCACCGCACCCGG | GAGATTGCAAGGGCGAATTC | | |
| | AAGGGCGAATTC (SEQ ID | (SEQ ID NO: 16) | | |
| | NO: 15) | | | |
| HeLa | GAATTCGCCCTTCCGGGCG | Bad sequencing | 203 | AluSc |
| heat | CGGTGGCACACGCTTGTAA | | | |
| shock_9 | TCCCATCACTTTGGGAGGT | | | |
| | CGAGGCGGGCGGATCACG | | | |
| | ATGTCAGGAGATCGAGACC | | | |
| | ATCCTGGCCAACATGGTGA | | | |
| | GGCCCCGTCTCTACTAAAA | | | |
| | ATACAAAAATTAGCTGGGG | | | |
| | GTGGTGGCGCGCGCTTGTA | | | |
| | GTCCCAGTTAGTTGGGAGG | | | |
| | CTGAGGCAGGAGAATCGCT | | | |
| | TGAACCCGTGAGAAGGAGA | | | |
| | TTGCAAGGGCGAATTC | | | |
| | (SEQ ID NO: 17) | | | |
| HeLa | AATTCGCCCTTGCAATCTCC | GAATTCGCCCTTCCGGGTGC | 203 | AluSc |
| heat | TTCTCACGGGTTCAAGCGA | GGTGGCACACGCTTGTAATC | | |
| shock_10 | TTCTCCTGCCTCAGCCTCC | CCAGCACTTTGGGAGGTCGA | | |
| | CAACTAACTGGGACTACAA | GGCGGGCGGATCACGAGGTC | | |
| | GCGCGCGCCACCACGCCC | AGGAGATCGAGACCATCCTG | | |
| | AGCTAATTTTTGTATTTTTAG | GCCAACATGGTGAAGCCCCG | | |
| | TAGAGACGGGGCTTCACCA | TCTCTACTAAAAATACAAAAAT | | |
| | TGTTGGCCAGGATGGTCTC | TAGCTGGGCGTGGTGGCGCG | | |
| | GATCTCCTGACCTCGTGAT | CGCTTGTAGTCCCAGTTAGTT | | |
| | CCGCCCGCCTCGACCTCCC | GGGAGGCTGAGGCAGGAGAA | | |
| | AAAGTGCTGGGATTACAAG | TCGCTTGAACCCGTGAGAAG | | |
| | CGTGTGCCACCGCACCCGG | GAGATTGCAAGGGCGAATTC | | |
| | AAGGGCGAATTC (SEQ ID | (SEQ ID NO: 19) | | |
| | NO: 18) | | | |
| HeLa | GAATTCGCCCTTCCGGGTG | GAATTCGCCCTTGCAATCTCC | 203 | AluSc |
| heat | CGGTGGCACACGCTTGTAA | TTCTCACGGGTTCAAGCGATT | | |
| shock_11 | TCCCAGCACTCTGGGAGGT | CTCCTGCCTCAGCCTCCCAAC | | |
| | CGAGGGGGGCGGATCACG | TAACTGGGACTACAAGCGCG | | |
| | AGGTCAGGAGATCGAGACC | CGCCACCACGCCCAGCTAAT | | |
| | ATCCTGGCCGGCATGGTGA | TTTTTGTATTTTTAGTAGAGAC | | |
| | GGCCCCGTCTCTACTAAAA | GGGGCCTCACCATGCCGGCC | | |
| | ATACAAAAAATTAGCTGGGC | AGGATGGTCTCGATCTCCTGA | | |
| | GTGGTGGCGCGCGCTTGTA | CCTCGTGATCCGCCCGCCTC | | |
| | GTCCCAGTTAGTTGGGAGG | GACCTCCCAGAGTGCTGGGA | | |
| | CTGAGGCAGGAGAATCGCT | TTACAAGCGTGTGCCACCGC | | |
| | TGAACCCGTGAGAAGGAGA | ACCCGGAAGGGCGAATTC | | |
| | TTGCAAGGGCGAATTC | (SEQ ID NO: 21) | | |
| | (SEQ ID NO: 20) | | | |
| HeLa | GAATTCGCCCTTGCAATCTC | GAATTCGCCCTTCCGGGTGC | 203 | AluSc |
| heat | CTTCTCACGGGTTCAAGCG | GGTGGCTCACGCTTGTAACC | | |
| shock_12 | ATTCTCCTGCCTCAGCCTC | CCAGCACTTTGGGAGGCCGA | | |
| | CCAACTAACTGGGACTACA | GGTGGGCGGATCACGAGGTC | | |
| | AGCGCGCGCCACCACGCC | AGGAGATCGAGACCATCCTG | | |
| | CAGCTAATTTTTTGTATTTTT | GCCAACATGGTGAAGCCCCG | | |
| | AGTAGAGACGGGGCTTCAC | TCTCTACTAAAAATACAAAAAA | | |
| | CATGTTGGCCAGGATGGTC | TTAGCTGGGCGTGGTGGCGC | | |
| | TCGATCTCCTGACCTCGTG | GCGCTTGTAGTCCCAGTTAGT | | |
| | ATCCGCCCACCTCGGCCTC | TGGGAGGCTGAGGCAGGAGA | | |
| | CCAAAGTGCTGGGGTTACA | ATCGCTTGAACCCGTGAGAA | | |
| | AGCGTGTGCCACCGCACCC | GGAGATTGCAAGGGCGAATT | | |
| | GGAAGGGCGAATTC (SEQ | C (SEQ ID NO: 23) | | |
| | ID NO: 22) | | | |
| HeLa | GAATTCGCCCTTGCAATCTC | GAATTCGCCCTTCCGGGTGC | 220 | AluSc8 |
| heat | CTTCTCACGGGTTCAAGCA | GGTGGCACACGCTTGTAATC | | |
| shock_13 | ATTCTCCTGCCTCAGCCTC | CCAGCACTTTGGGAGGCCAA | | |
| | CAAGTACCTGGGACTACAG | GGCGGGCAGATCATGAGGTC | | |
| | GCGCTGGCCACCATGCCCA | AGGAAATTGAGACCAGCCTG | | |
| | GCTAATTTTCTGTATTTTTA | GCTAACATGGTGAAACTCTGT | | |
| | GTAGAGACAGAGTTTCACC | CTCTACTAAAAATACAGAAAA | | |
| | ATGTTAGCCAGGCTGGTCT | TTAGCTGGGCATGGTGGCCA | | |
| | CAATTTCCTGACCTCATGAT | GCGCCTGTAGTCCCAGGTAC | | |
| | CTGCCCGCCTTGGCCTCCC | TTGGAGGCTGAGGCAGGAGA | | |
| | AAAGTGCTGGGATTACAAG | ATTGCTTGAACCCGTGAGAAG | | |
| | CGTGTGCCACCGCACCCGG | GAGATTGCAAGGGCGAATTC | | |
| | AAGGGCGAATTC (SEQ ID | (SEQ ID NO: 25) | | |
| | NO: 24) | | | |
| HeLa | GAATTCGCCCTTGCAATCTC | GAATTCGCCCTTCCGGGTGC | 203 | AluSc |
| heat | CTTCTCACGGGTTCAAGCG | GGTGGCACACGCTTGTAATC | | |
| shock_14 | ATTCTCCTGCCTCAGCCTC | CCAGCACTTTGGGAGGTCGA | | |
| | CCAACTAACTGGGACTACA | GGCGGGCGGATCACGAGGTC | | |
| | AGCGCGCGCCACCACGCC | AGGAGATCGAGACCATCCTG | | |
| | CAGCTAATTTTTGTATTTTTT | GCCAACATGGTGAAGCCCCG | | |
| | TTAGTAGAGACGGGGCTTC | TCTCTACTAAAAAAAATACAAA | | |
| | ACCATGTTGGCCAGGATGG | AATTAGCTGGGCGTGGTGGC | | |
| | TCTCGATCTCCTGACCTCGT | GCGCGCTTGTAGTCCCAGTTA | | |
| | GATCCGCCCGCCTCGACCT | GTTGGGAGGCTGAGGCAGGA | | |
| | CCCAAAGTGCTGGGATTAC | GAATCGCTTGAACCCGTGAG | | |
| | AAGCGTGTGCCACCGCACC | AAGGAGATTGCAAGGGCGAA | | |
| | CGGAAGGGCGAATTC (SEQ | TTC (SEQ ID NO: 27) | | |
| | ID NO: 26) | | | |
| HeLa | GAATTCGCCCTTGCAATCTC | GAATTCGCCCTTCCGGGTGC | 203 | AluSc |
| heat | CTTCTCACGGGTTCAAGCG | GGTGGCCACACGCTTGTAAT | | |
| shock_15 | ATTCTCCTGCCTCAGCCTC | CCCAGCACTTTGGGAGGTCG | | |
| | CCAACTAACTGGGACTACA | AGGCGGGCGGATCACGAGGT | | |
| | AGCGCGCGCCACCACGCC | CAGGAGATCGAGACCATCCT | | |
| | CAGCTAATTTTTGTATTTTTA | GGCCAACATGGTGAAGCCCC | | |
| | GTAGAGACGGGGCTTCACC | GTCTCTACTAAAAATACAAAA | | |
| | ATGTTGGCCAGGATGGTCT | ATTAGCTGGGCGTGGTGGCG | | |
| | CGATCTCCTGACCTCGTGA | CGCGCTTGTAGTCCCAGTTAG | | |
| | TCCGCCCGCCTCGACCTCC | TTGGGAGGCTGAGGCAGGAG | | |
| | CAAAGTGCTGGGATTACAA | AATCGCTTGAACCCGTGAGAA | | |
| | GCGTGTGCCACCGCACCCG | GGAGATTGCAAGGGCGAATT | | |
| | GAAGGGCGAATTC (SEQ ID | C (SEQ ID NO: 29) | | |
| | NO: 28) | | | |
| HeLa | GAATTCGCCCTTCCGGGTG | GAATTCGCCCTTGCAATCTCC | 203 | AluSc |
| heat | CGGTGGCACACGCTTGTAA | TTCTCACGGGTTCAAGCGATT | | |
| shock_16 | TCCCAGCACTTTGGGAGGT | CTCCTGCCTCAGCCTCCCAAC | | |
| | CGAGGCGGGCGGATCACG | TAACTGGGACTACAAGCGCG | | |
| | AGGTCAGGAGATCGAGACC | CGCCACCACGCCCAGCTAAT | | |
| | ATCCTGGCCAACATGGTGA | TTTTGTATTTTTAGTAGAGACG | | |
| | AGCCCCGTCTCTACTAAAAA | GGGCTTCACCATGTTGGCCA | | |
| | TACAAAAATTAGCTGGGCG | GGATGGTCTCGATCTCCTGAC | | |
| | TGGTGGCGCGCGCTTGTAG | CTCGTGATCCGCCCGCCTCG | | |
| | TCCCAGTTAGTTGGGAGGC | ACCTCCCAAAGTGCTGGGATT | | |
| | TGAGGCAGGAGAATCGCTT | ACAAGCGTGTGCCACCGCAC | | |
| | GAACCCGTGAGAAGGAGAT | CCGGAAGGGCGAATTC (SEQ | | |
| | TGCAAGGGCGAATTC (SEQ | ID NO: 31) | | |
| | ID NO: 30) | | | |
| HeLa | GAATTCGCCCTTGCAATCTC | GAATTCGCCCTTCCGGGTGC | 221 | AluSx |
| heat | CTTCTCACGGGTTCAAGAG | GGTGGCCCACGCTTGTAATC | | |
| shock_17 | ATTCTCCTGCTGCCGCCTC | CCAGCACTTTGGGAGGTCGA | | |
| | CCACGTAGCTGAGACTACA | GACGGGTGGATCACCTCAGG | | |
| | GGTGTGTACCACCACGCCT | TCAGAGTTCAAGAGCAGCCC | | |
| | AGCTATATTTTGCATTTTTA | CGCCATCAAGACAAAACCTCC | | |
| | GTAGAGAGGAGGTTTTGTC | TCTCTACTAAAAATGCAAAATA | | |
| | TTGATGGCGGGGCTGCTCT | TAGCTAGGCGTGGTGGTACA | | |
| | TGAACTCTGACCTGAGGTG | CACCTGTAGTCTCAGCTACGT | | |
| | ATCCACCCGTCTCGACCTC | GGGAGGCGGCAGCAGGAGA | | |
| | CCAAAGTGCTGGGATTACA | ATCTCTTGAACCCGTGAGAAG | | |
| | AGCGTGGGCCACCGCACC | GAGATTGCAAGGGCGAATTC | | |
| | CGGAAGGGCGAATTC (SEQ | (SEQ ID NO: 33) | | |
| | ID NO: 32) | | | |
| HeLa | GAATTCGCCCTTGCAATCTC | GAATTCGCCCTTCCGGGTGC | 220 | AluSc8 |
| heat | CTTCTCACGGGTTCAAGCA | GGTGGCACACGCTTGTAATC | | |
| shock_18 | ATTCTCCTGCCTCAGCCTC | CCAGCACTTTGGGAGGCCAA | | |
| | CAAGTACCTGGGACTACAG | GGCGGGCAGATCATGAGGTC | | |
| | GCGCTGGCCACCATGCCCA | AGGAAATTGAGACCAGCCTG | | |
| | GCTAATTTTCTGTATTTTTA | GCTAACATGGTGAAACTCTGT | | |
| | GTAGAGACAGAGTTTCACC | CTCTACTAAAAATACAGAAAA | | |
| | ATGTTAGCCAGGCTGGTCT | TTAGCTGGGCATGGTGGCCA | | |
| | CAATTTCCTGACCTCATGAT | GCGCCTGTAGTCCCAGGTAC | | |
| | CTGCCCGCCTTGGCCTCCC | TTGGAGGCTGAGGCAGGAGA | | |
| | AAAGTGCTGGGATTACAAG | ATTGCTTGAACCCGTGAGAAG | | |
| | CGTGTGCCACCGCACCCGG | GAGATTGCAAGGGCGAATTC | | |
| | AAGGGCGAATTC (SEQ ID | (SEQ ID NO: 35) | | |
| | NO: 34) | | | |
| HeLa | GAATTCGCCCTTGCAATCTC | GAATTCGCCCTTCCGGGTGC | 203 | AluSc |
| heat | CTTCTCACGGGTTCAAGCG | GGTGGCACACGCTTGTAATC | | |
| shock_19 | ATTCTCCTGCCTCAGCCTC | CCAGCACTTTGGGAGGTCGA | | |
| | CCAACTAACTGGGACTACA | GGCGGGCGGATCACGAGGTC | | |
| | AGCGCGCGCCACCACGCC | AGGAGATCGAGACCATCCTG | | |
| | CAGCTAATTTTTGTATTTTTA | GCCAACATGGTGAAGCCCCG | | |
| | GTAGAGACGGGGCTTCACC | TCTCTACTAAAAATACAAAAAT | | |
| | ATGTTGGCCAGGATGGTCT | TAGCTGGGCGTGGTGGCGCG | | |
| | CGATCTCCTGACCTCGTGA | CGCTTGTAGTCCCAGTTAGTT | | |
| | TCCGCCCGCCTCGACCTCC | GGGAGGCTGAGGCAGGAGAA | | |
| | CAAAGTGCTGGGATTACAA | TCGCTTGAACCCGTGAGAAG | | |
| | GCGTGTGCCACCGCACCCG | GAGATTGCAAGGGCGAATTC | | |
| | GAAGGGCGAATTC (SEQ ID | (SEQ ID NO: 37) | | |
| | NO: 36) | | | |
| HeLa | GAATTCGCCCTTGCAATCTC | GAATTCGCCCTTCCGGGTGC | 203 | AluSc |
| heat | CTTCTCACGGGTTCAAGCG | GGTGGCACACGCTTGTAATC | | |
| shock_20 | ATTCTCCTGCCTCAGCCTC | CCAGCACTTTGGGAGGTCGA | | |
| | CCAACTAACTGGGACTACA | GGCGGGCGGATCACGAGGTC | | |
| | AGCGCGCGCCACCACGCC | AGGAGATCGAGACCATCCTG | | |
| | CAGCTAATTTTTGTATTTTTA | GCCAACATGGTGAAGCCCCG | | |
| | GTAGAGACGGGGCTTCACC | TCTCTACTAAAAATACAAAAAT | | |
| | ATGTTGGCCAGGATGGTCT | TAGCTGGGCGTGGTGGCGCG | | |
| | CGATCTCCTGACCTCGTGA | CGCTTGTAGTCCCAGTTAGTT | | |
| | TCCGCCCGCCTCGACCTCC | GGGAGGCTGAGGCAGGAGAA | | |
| | CAAAGTGCTGGGATTACAA | TCGCTTGAACCCGTGAGAAG | | |
| | GCGTGTGCCACCGCACCCG | GAGATTGCAAGGGCGAATTC | | |
| | GAAGGGCGAATTC (SEQ ID | (SEQ ID NO: 39) | | |
| | NO: 38) | | | |
| HeLa | GAATTCGCCCTTGCAATCTC | GAATTCGCCCTTCCGGGTGC | 203 | AluSc |
| heat | CTTCTCACGGGTTCAAGCG | GGTGGCACACGCTTGTAATC | | |
| shock_21 | ATTCTCCTGCCTCAGCCTC | CCAGCGCTTTGGGAGGTCGA | | |
| | CCAACTAACCGGGACCACA | GGCGGGCGGATCACGAGGTC | | |
| | AGCGCGCGCCACCACGCC | AGGAGATCGAGACCATCCTG | | |
| | CAGCCAATTTTTGTATTTTT | GCCAACATGGTGAAGCCCCG | | |
| | AGTAGAGACGGGGCTTCAC | TCTCTACTAAAAATACAAAAAT | | |
| | CATGTTGGCCAGGATGGTC | TGGCTGGGCGTGGTGGCGCG | | |
| | TCGATCTCCTGACCTCGTG | CGCTTGTGGTCCCGGTTAGTT | | |
| | ATCCGCCCGCCTCGACCTC | GGGAGGCTGAGGCAGGAGAA | | |
| | CCAAAGCGCTGGGATTACA | TCGCTTGAACCCGTGAGAAG | | |
| | AGCGTGTGCCACCGCACCC | GAGATTGCAAGGGCGAATTC | | |
| | GGAAGGGCGAATTC (SEQ | (SEQ ID NO: 41) | | |
| | ID NO: 40) | | | |
| | | | | hg38/ |
| Clone | | | | Human |
| number | Sequencing with T3 primer | Sequencing with T7 primer | Span | Alu |
| Heat | Bad sequencing | GAATTCGCCCTTGTGCGGTG | 217 | AluSx |
| shock_22 | | GCACACGCTTGTAATCCCAGC | | |
| | | ACTTTGGGAGGTCGAGACGG | | |
| | | GTGGATCACCTCAGGTCAGA | | |
| | | GTTCAAGAGCAGCCCCGCCA | | |
| | | TCAAGACAAAACCTCCTCTCT | | |
| | | ACTAAAAATGCAAAATATAGC | | |
| | | TAGGCGTGGTGGTACACACC | | |
| | | TGTAGTCTCAGCTACGTGGGA | | |
| | | GGCGGCAGCAGGAGAATCTC | | |
| | | TTGAACCCGTGAGAAGGAGA | | |
| | | TTGCAAGGGCGAATTC (SEQ | | |
| | | ID NO: 42) | | |
| Heat | GAATTCGCCCTTGCA_TCTC | GAATTCGCCCTTCCGGGTGC | 217 | AluSx |
| shock_23 | CTTCTCACGGGTTCAAGAG | GGTGGCACACGCTTGTAATC | | |
| | ATTCTCCTGCTGCCGCCTC | CCAGCACTTTGGGAGGTCGA | | |
| | CCACGTAGCTGAGACTACA | GACGGGTGGATCACCTCAGG | | |
| | GGTGTGTACTACCACGCCT | TCAGAGTTCAAGAGCAGCCC | | |
| | AGCTATATTTTGCATTTTTA | CGCCATCAAGACAAAACCTCC | | |
| | GTAGAGAGGAGGTTTTGTC | TCTCTACTAAAAATGCAAAATA | | |
| | TTGATGGCGGGGCTGCTCT | TAGCTAGGCGTGGTAGTACA | | |
| | TGAACTCTGACCTGAGGTG | CACCTGTAGTCTCAGCTACGT | | |
| | ATCCACCCGTCTCGACCTC | GGGAGGCGGCAGCAGGAGA | | |
| | CCAAAGTGCTGGGATTACA | ATCTCTTGAACCCGTGAGAAG | | |
| | AGCGTGTGCCACCGCACCC | GAGATTGCAAGGGCGAATTC | | |
| | GGAAGGGCGAATTC (SEQ | (SEQ ID NO: 44) | | |
| | ID NO: 43) | | | |
| Heat | GAATTCGCCCTTGCAT_CTC | GAATTCGCCCTTGGTGCGGT | 218 | AluSx |
| shock_24 | CTTCTCACGGGTTCAAGAG | GGCACACGCTTGTAATCCCA | | |
| | ATTCTCCTGCTGCCGCCTC | GCACTTTGGGAGGTCGAGAC | | |
| | CCACGTAGTCTGAGACTAC | GGGTGGATCACCTCAGGTCA | | |
| | AGGTGTGTACCACCACGCC | GAGTTCAAGAGCAGCCCCGC | | |
| | TAGCTATATTTTGCATTTTTA | CATCAAGATAAAACCTCCTCT | | |
| | GTAGAGAGGAGGTTTTATC | CTACTAAAAATGCAAAATATA | | |
| | TTGATGGCGGGGCTGCTCT | GCTAGGCGTGGTGGTACACA | | |
| | TGAACTCTGACCTGAGGTG | CCTGTAGTCTCAGCTACGTGG | | |
| | ATCCACCCGTCTCGACCTC | GAGGCGGCAGCAGGAGAATC | | |
| | CCAAAGTGCTGGGATTACA | TCTTGAACCCGTGAGAAGGA | | |
| | AGCGTGTGCCACCGCACCA | GATTGCAAGGGCGAATTC | | |
| | AGGGCGAATTC (SEQ ID NO: | (SEQ ID NO: 46) | | |
| | 45) | | | |
| Heat | GATTCCCTTGCAAT_CTCCT | GAATTCGCCCTTCCGGGTGC | 22 | AluSx |
| shock_25 | TCTCACGGGTTCAAGAGAT | GGTGGCACACGCTTGTAATC | | |
| | TCTCCTGCTGCCGCCTCCC | CCAGCACTTTGGGAGGTCGA | | |
| | ACGCAGCTGAGACTACAGG | GACGGGCGGATCACCTCAGG | | |
| | TGTGTACCACCACGCCTAG | TCAGAGTTCAAGAGCAGCCC | | |
| | CTATATTTTGCATTTTTAGTA | CGCCATCAAGACAAAACCTCC | | |
| | GAGAGGAGGTTTTGTCTTG | TCTCTACTAAAAATGCAAAATA | | |
| | ATGGCGGGGCTGCTCTTGA | TAGCTAGGCGTGGTGGTACA | | |
| | ACTCTGACCTGAGGTGATC | CACCTGTAGTCTCAGCTACGT | | |
| | CGCCCGCCTCGACCTCCCA | GGGAGGCGGCAGCAGGAGA | | |
| | AGCTGCTGTTATTACGATCC | ATCTCTTGAACCCGTGAGAAG | | |
| | CGTGCCACCGCCATAGTGA | GAGATTGCAAGGGCGAATTC | | |
| | GGACGACTCCGCCTCAACC | (SEQ ID NO: 48) | | |
| | TTCAATTC (SEQ ID NO: 47) | | | |
| Heat | Bad sequencing | GAATTCGCCCTTGGGTGCGG | 214 | AluSx3 |
| shock_26 | | TGGCACACGCTTGTAATCCCA | | |
| | | GCACTTTGGGAGGCCAAGGC | | |
| | | GGGCAGATCATGAGGTCAGG | | |
| | | AAATTGAGACCAGCCTGGCTA | | |
| | | ACATGGTGAAACTCTGTCTCT | | |
| | | ACTAAAAATACAGAAAATTAG | | |
| | | CTGGGCATGGTGGCCAGCGC | | |
| | | CTGTAGTCCCAGGTACTTGGA | | |
| | | GGCTGAGGCAGGAGAATTGC | | |
| | | TTGAACCCGTGAGAAGGAGA | | |
| | | TTGCAAGGGCGAATTC (SEQ | | |
| | | ID NO: 49) | | |
| Heat | GAATTCGCCCTTGCA_TCTC | GAATTCGCCCTTCCGGGTGC | 73 | AluSx |
| shock_27 | CTTCTCACGGGTTCAAGCG | GGTGGCACACGCTTGTGGTC | | |
| | ATTCTCCTGCCTCAGCCTC | CCAGCTATTCAGGAGGCTGA | | |
| | CTGAATAGCTGGGACCACA | GGCCAGGAGAATCGCTTGAA | | |
| | AGCGTGTGCCACCGCACCC | CCCGTGAGAAGGAGATTGCA | | |
| | GGAAGGGCGAATTC (SEQ | AGGGCGAATTC (SEQ ID NO: | | |
| | ID NO: 50) | 51) | | |
| Heat | GAATTCGCCCTTGCA_TCTC | GAATTCGCCCTTCCGGGTGC | 203 | AluSc |
| shock_28 | CTTCTCACGGGTTCAAGCG | GGTGGCACACGCTTGTAATC | | |
| | ATTCTCCTGCCTCAGCCTC | CCAGCACTTTGGGAGGTCGA | | |
| | CCAACTAACTGGGACTACA | GGCCGGGCGGATCACGAGGT | | |
| | AGCGCGCGCCACCACGCC | CAGGAGATCGAGACCATCCT | | |
| | CAGCTAATTTTTGTATTTTTA | GGCCAACATGGTGAAGCCCC | | |
| | GTAGAGACGGGGCTTCACC | GTCTCTACTAAAAATACAAAA | | |
| | ATGTTGGCCAGGATGGTCT | ATTAGCTGGGCGTGGTGGCG | | |
| | CGATCTCCTGACCTCGTGA | CGCGCTTGTAGTCCCAGTTAG | | |
| | TCCGCCCGCCTCGACCTCC | TTGGGAGGCTGAGGCAGGAG | | |
| | CAAAGTGCTGGGATTACAA | AATCGCTTGAACCCGTGAGAA | | |
| | GCGTGTGCCACCGCACCCG | GGAGATTGCAAGGGCGAATT | | |
| | GAAGGGCGAATTC (SEQ ID | C (SEQ ID NO: 53) | | |
| | NO: 52) | | | |
| Heat | GAATTCGCCCTTGCA_TCTC | GAATTCGCCCTTCCGGGTGC | 221 | AluSx |
| shock_29 | CTTCTCACGGGTTCAAGAG | GGTGGCACACGCTTGTAATC | | |
| | ATCCTCCTGCTGCCGCCTC | CCAGCACTTTGGGAGGTCGA | | |
| | CCACGTAGCTGAGACTACA | GACGGGTGGATCACCTCAGG | | |
| | GGTGTGTACCACCACGCCT | TCAGAGTCCAAGAGCAGCCC | | |
| | AGCTATATTTTGCATTTTTA | CGCCATCAAGACAAAACCTCC | | |
| | GTAGAGAGGAGGTTTTGTC | TCTCTACTAAAAATGCAAAATA | | |
| | TTGATGGCGGGGCTGCTCT | TAGCTAGGCGTGGTGGTACA | | |
| | TGGACTCTGACCTGAGGTG | CACCTGTAGTCTCAGCTACGT | | |
| | ATCCACCCGTCTCGACCTC | GGGAGGCGGCAGCAGGAGG | | |
| | CCAAAGTGCTGGGATTACA | ATCTCTTGAACCCGTGAGAAG | | |
| | AGCGTGTGCCACCGCACCC | GAGATTGCAAGGGCGAATTC | | |
| | GGAAGGGCGAATTC (SEQ | (SEQ ID NO: 55) | | |
| | ID NO: 54) | | | |
| Heat | GAATTCGCCCTTCGTGCGG | GAATTCGCCCTTGCA_TCTCC | 217 | AluSx |
| shock_30 | TGGCACACGCTTGTAATCC | TTCTCACGGGTTCAAGAGATT | | |
| | CAGCACTTTGGGAGGTCGA | CTCCTGCTGCCGCCTCCCAC | | |
| | GACGGGTGGATCGCCTCAG | GTAGTCTGAGACTACAGGTGT | | |
| | GTCAGAGTTCAAGAGCAGC | GAACCACCACGCCTAGCTATA | | |
| | CCCGCCATCAAGACAAAAC | TTTTGCATTTTTAGTAGAGAG | | |
| | CTCCTCTCTACTAAAAATGC | GAGGTTTTGTCTTGATGGCGG | | |
| | AAAATATAGCTAGGCGTGG | GGCTGCTCTTGAACTCTGACC | | |
| | TGGTTCACACCTGTAGTCTC | TGAGGCGATCCACCCGTCTC | | |
| | AGCTACGTGGGAGGCGGC | GACCTCCCAAAGTGCTGGGA | | |
| | AGCAGGAGAATCTCTTGAA | TTACAAGCGTGTGCCACCGC | | |
| | CCCGTGAGAAGGAGATTGC | ACGAAGGGCGAATTC (SEQ ID | | |
| | AAGGGCGAATTC (SEQ ID | NO: 57) | | |
| | NO: 56) | | | |
| Heat | GAATTCGCCCTTGGGTGCG | AATTCGCCCTTGCA_TCTCCT | 173 | AluSx4 |
| shock_31 | GTGGCACACGCTTGTAATA | TCTCACGGGTTCAAGCTATTC | | |
| | CCAGCACTTTGGGAGGCCA | TCCTGCCTCAGCCTCCCGAG | | |
| | AGGTGGGCGGATCACCTGA | TAGCTGGGACTACAAGGGAT | | |
| | GGTCAGGAGTTCAAGACCA | CTGCCACCACGCCCGGCTAA | | |
| | GCCTGGCCAATATGGTGAA | TTTTTGTAATTTTAGTAGAGAC | | |
| | AGTCCGTCTCTACTAAAATT | GGACTTTCACCATATTGGCCA | | |
| | ACAAAAATTAGCCGGGCGT | GGCTGGTCTTGAACTCCTGAC | | |
| | GGTGGCAGATCCCTTGTAG | CTCAGGTGATCCGCCCACCTT | | |
| | TCCCAGCTACTCGGGAGGC | GGCCTCCCAAAGTGCTGGTA | | |
| | TGAGGCAGGAGAATAGCTT | TTACAAGCGTGTGCCACCGC | | |
| | GAACCCGTGAGAAGGAGAT | ACCAAGGGCGAATTC (SEQ ID | | |
| | TGCAAGGGCGAATTC (SEQ | NO: 59) | | |
| | ID NO: 58) | | | |
| Heat | GAATTCGCCCTTGCAATCTC | GAATTCGCCCTTCCGGGTGC | 221 | AluSx |
| shock_32 | CTTCTCACGGGTTCAAGAG | GGTGGCACACGCTTGTAATC | | |
| | ACTCTCCTGCTGCCGCCTC | CCAGCACTTTGGGAGGTCGA | | |
| | CCACGTAGCTGAGACTACA | GACGGGTGGATCACCTCAGG | | |
| | GGTGTGTACCACCACGCCT | TCAGAGTTCAAGAGCAGCCC | | |
| | AGCTATATTTTGCATTTTTA | CGCCATCAAGACAAAACCTCC | | |
| | GTAGAGAGGAGGTTTTGTC | TCTCTACTAAAAATGCAAAATA | | |
| | TTGATGGGGGGCTGCTCT | TAGCTAGGCGTGGTGGTACA | | |
| | TGAACTCTGACCTGAGGTG | CACCTGTAGTCTCAGCTACGT | | |
| | ATCCACCCGTCTCGACCTC | GGGAGGCGGCAGCAGGAGA | | |
| | CCAAAGTGCTGGGATTACA | GTCTCTTGAACCCGTGAGAAG | | |
| | AGCGTGTGCCACCGCACCC | GAGATTGCAAGGGCGAATTC | | |
| | GGAAGGGCGAATTC (SEQ | (SEQ ID NO: 61) | | |
| | ID NO: 60) | | | |
| Heat | GAATTCGCCCTTCCGGGTG | ATTCGCCCTTGCA_TCTCCTT | 221 | AluSx |
| shock_33 | CGGTGGCACACGCTTGTAA | CTCACGGGTTCAAGAGATTCT | | |
| | TCCCAGCACTTTGGGAGGT | CCTGCTGCCGCCTCCCACGT | | |
| | CGAGACGGGTGGATCACCT | AGCTGAGACTACAGGTGTGTA | | |
| | CAGGTCAGAGTTCAAGAGC | CCACCACGCCTAGCTATATTT | | |
| | AGCCCCGCCATCAAGACAA | TGCATTTTTAGTAGAGAGGAG | | |
| | AACCTCCTCTCTACTAAAAA | GTTTTGTCTTGATGGCGGGG | | |
| | TGCAAAATATAGCTAGGCG | CTGCTCTTGAACTCTGACCTG | | |
| | TGGTGGTACACACCTGTAG | AGGTGATCCACCCGTCTCGA | | |
| | TCTCAGCTACGTGGGAGGC | CCTCCCAAAGTGCTGGGATTA | | |
| | GGCAGCAGGAGAATCTCTT | CAAGCGTGTGCCACCGCACC | | |
| | GAACCCGTGAGAAGGAGAT | CGGAAGGGCGAATTC (SEQ ID | | |
| | TGCAAGGGCGAATTC (SEQ | NO: 63) | | |
| | ID NO: 62) | | | |
| Heat | Bad sequencing | GAATTCGCCCTTGCAATCTCC | 374 | AluSx |
| shock_34 | | TTCTCACGGGTTCAAGCGATT | | |
| | | CTCCTGCCTCAGCCTCCTGA | | |
| | | GTAGCTGGGATTACAAGCGT | | |
| | | GTGCCACCGCACCCGGAAGG | | |
| | | GCGAATTC (SEQ ID NO: 64) | | |
| Heat | GAATTCGCCCTTGCAATCTC | GAATTCGCCCTTCCGGGTGC | 221 | AluSx |
| shock_35 | CTTCTCACGGGTTCAAGAG | GGTGGCACACGCTTGTAATC | | |
| | ATTCTCCTGCTGCCGCCTC | CCAGCACTTTGGGAGGTCGA | | |
| | CCACGTAGCTGAGACTACA | GACGGGTGGATCACCTCAGG | | |
| | GGTGTGTACCACCACGCCT | TCAGAGTTCGAGAGCAGCCC | | |
| | AGCTATATTTTGCATTTTTA | CGCCATCAAGACAAAACCTCC | | |
| | GTAGAGAGGAGGTTTTGTC | TCTCTACTAAAAATGCAAAATA | | |
| | TTGATGGCGGGGCTGCTCT | TAGCTAGGCGTGGTGGTACA | | |
| | CGAACTCTGACCTGAGGTG | CACCTGTAGTCTCAGCTACGT | | |
| | ATCCACCCGTCTCGACCTC | GGGAGGCGGCAGCAGGAGA | | |
| | CCAAAGTGCTGGGATTACA | ATCTCTTGAACCCGTGAGAAG | | |
| | AGCGTGTGCCACCGCACCC | GAGATTGCAAGGGCGAATTC | | |
| | GGAAGGGCGAATTC (SEQ | (SEQ ID NO: 66) | | |
| | ID NO: 65) | | | |
| Heat | GAATTCGCCCTTGCAATCTC | ATTCGCCCTTCCGGGTGCGG | 221 | AluSx |
| shock_36 | CTTCTCACGGGTTCAAGAG | TGGCACACGCTTGTAATCCCA | | |
| | ATTCTCCTGCTGCCGCCTC | GCACTTTGGGAGGCCGAGAC | | |
| | CCACGTAGCTGAGACTACA | GGGTGGATCACCTCAGGTCA | | |
| | GGTGTGTACCACCACGCCT | GAGTTCAAGAGCAGCCCCGC | | |
| | AGCTATATTTTGCATTTTTA | CATCAAGACAAAACCTCCTCT | | |
| | GTAGAGAGGAGGTTTTGTC | CTACTAAAAATGCAAAATATA | | |
| | TTGATGGCGGGGCTGCTCT | GCTAGGCGTGGTGGTACACA | | |
| | TGAACTCTGACCTGAGGTG | CCTGTAGTCTCAGCTACGTGG | | |
| | ATCCACCCGTCTCGGCCTC | GAGGCGGCAGCAGGAGAATC | | |
| | CCAAAGTGCTGGGATTACA | TCTTGAACCCGTGAGAAGGA | | |
| | AGCGTGTGCCACCGCACCC | GATTGCAAGGGCGAATTC | | |
| | GGAAGGGCGAATTC (SEQ | (SEQ ID NO: 68) | | |
| | ID NO: 67) | | | |
| Heat | GAATTCGCCCTTGGGTGCG | ATTCGCCCTTGCAATCTCCTT | 219 | AluSx1 |
| shock_37 | GTGGCACACGCTTGTAATC | CTCACGGGTTCAAGCGATTCT | | |
| | CCAGCACTTTGGGAGGCCG | CCTGCCTCAGTCTCCCGAGTA | | |
| | AGGTGAGCGGATCATGAGG | GCTGGTATTACAGGCGCCTG | | |
| | TCAGGAGTTTGAGACCAGC | CCACCATGCCCAGCTAATTTT | | |
| | CTGGCCAACACAGTGAAAA | TGTACTTTTAGTAGACGGGTT | | |
| | CCCGTCTACTAAAAGTACAA | TTCACTGTGTTGGCCAGGCTG | | |
| | AAATTAGCTGGGCATGGTG | GTCTCAAACTCCTGACCTCAT | | |
| | GCAGGCGCCTGTAATACCA | GATCCGCTCACCTCGGCCTC | | |
| | GCTACTCGGGAGACTGAGG | CCAAAGTGCTGGGATTACAAG | | |
| | CAGGAGAATCGCTTGAACC | CGTGTGCCACCGCACCAAGG | | |
| | CGTGAGAAGGAGATTGCAA | GCGAATTC (SEQ ID NO: 70) | | |
| | GGGCGAATTC (SEQ ID NO: | | | |
| | 69) | | | |
| Heat | GAATTCGCCCTTGCA_TCTC | ATTCGCCCTTGGGTGCGGTG | 198 | AluSc |
| shock_38 | CTTCTCACGGGTTCAAGCG | GCACACGCTTGTAATCCCAGC | | |
| | ATTCTCCTGCCTCAGCCTC | ACTTTGGGAGGTCGAGGCGG | | |
| | CCAACTAACTGGGACTACA | GCGGATCACGAGGTCAGGAG | | |
| | AGCGCGCGCCACCACGCC | ATCGAGACCATCCTGGCCAA | | |
| | CAGCTAATTTTTGTATTTTTA | CATGGTGAAGCCCCGTCTCTA | | |
| | GTAGAGACGGGGCTTCACC | CTAAAAATACAAAAATTAGCT | | |
| | ATGTTGGCCAGGATGGTCT | GGGCGTGGTGGCGCGCGCTT | | |
| | CGATCTCCTGACCTCGTGA | GTAGTCCCAGTTAGTTGGGA | | |
| | TCCGCCCGCCTCGACCTCC | GGCTGAGGCAGGAGAATCGC | | |
| | CAAAGTGCTGGGATTACAA | TTGAACCCGTGAGAAGGAGA | | |
| | GCGTGTGCCACCGCACCCA | TTGCAAGGGCGAATTC (SEQ | | |
| | AGGGCGAATTC (SEQ ID NO: | ID NO: 72) | | |
| | 71) | | | |
| Heat | GAATTCGCCCTTGCAATCTC | GAATTCGCCCTTGGGTGCGG | 219 | AluSx |
| shock_39 | CTTCTCACGGGTTCAAGAG | TGGCACACGCTTGTAATCCCA | | |
| | ATTCTCCTGCTGCCGCCTC | GCACTTTGGGAGGTCGAGAC | | |
| | CCACGTAGCTGAGACTACA | GGGTGGATCACCTCAGGTCA | | |
| | GGTGTGTACCACCACGCCT | GAGTTCAAGAGCAGCCCCGC | | |
| | AGCTATATTTTGCATTTTTA | CATCAAGACAAAACCTCCTCT | | |
| | GTAGAGAGGAGGTTTTGTC | CTACTAAAAATGCAAAATATA | | |
| | TTGATGGCGGGGCTGCTCT | GCTAGGCGTGGTGGTACACA | | |
| | TGAACTCTGACCTGAGGTG | CCTGTAGTCTCAGCTACGTGG | | |
| | ATCCACCCGTCTCGACCTC | GAGGCGGCAGCAGGAGAATC | | |
| | CCAAAGTGCTGGGATTACA | TCTTGAACCCGTGAGAAGGA | | |
| | AGCGTGTGCCACCGCACCA | GATTGCAAGGGCGAATTC | | |
| | AGGGCGAATTC (SEQ ID NO: | (SEQ ID NO: 74) | | |
| | 73) | | | |
| Heat | GAATTCGCCCTTGCA_TCTC | GAATTCGCCCTTGGGTGCGG | 219 | AluSx |
| shock_40 | CTTCTCACGGGTTCAAGAG | TGGCACACGCTTGTAATCCCA | | |
| | ATTCTCCTGCTGCCGCCTC | GCACTTTGGGAGGTCGAGAC | | |
| | CCACGTAGCTGAGACTACA | GGGTGGATCACCTCAGGTCA | | |
| | GGTGTGTACCACCACGCCT | GAGTTCAAGAGCAGCCCCGC | | |
| | AGCTATATTTTGCATTTTTA | CATCAAGACAAAACCTCCTCT | | |
| | GTGGAGAGGAGGTTTTGTC | CCACTAAAAATGCAAAATATA | | |
| | TTGATGGCGGGGCTGCTCT | GCTAGGCGTGGTGGTACACA | | |
| | TGAACTCTGACCTGAGGTG | CCTGTAGTCTCAGCTACGTGG | | |
| | ATCCACCCGTCTCGACCTC | GAGGCGGCAGCAGGAGAATC | | |
| | CCAAAGTGCTGGGATTACA | TCTTGAACCCGTGAGAAGGA | | |
| | AGCGTGTGCCACCGCACCA | GATTGCAAGGGCGAATTC | | |
| | AGGGCGAATTC (SEQ ID NO: | (SEQ ID NO: 76) | | |
| | 75) | | | |
| Heat | GAATTCGCCCTTGGGTGCG | ATTCGCCCTTGCAATCTCCTT | 219 | AluSx |
| shock_ 41 | GTGGCACACGCTTGTAATC | CTCACGGGTTCAAGAGATTCT | | |
| | CCAGCACTTTGGGAGGTCG | CCTGCTGCCGCCTCCCACGT | | |
| | AGACGGGTGGATCACCTCA | AGCTGAGACTACAGGTGTGTA | | |
| | GGTCAGAGTTCAAGAGCAG | CCACCACGCCTAGCTATATTT | | |
| | CCCCGCCATCAAGACAAAA | TGCATTTTTAGTAGAGAGGAG | | |
| | CCTCCTCTCTACTAAAAATG | GTTTTGTCTTGATGGCGGGG | | |
| | CAAAATATAGCTAGGCGTG | CTGCTCTTGAACTCTGACCTG | | |
| | GTGGTACACACCTGTAGTC | AGGTGATCCACCCGTCTCGA | | |
| | TCAGCTACGTGGGAGGCGG | CCTCCCAAAGTGCTGGGATTA | | |
| | CAGCAGGAGAATCTCTTGA | CAAGCGTGTGCCACCGCACC | | |
| | ACCCGTGAGAAGGAGATTG | CAAGGGCGAATTC (SEQ ID | | |
| | CAAGGGCGAATTC (SEQ ID | NO: 78) | | |
| | NO: 77) | | | |

### Example 3

### Quantification and Statistical Analysis of Mir-498 (46) Cistron Detection and Antiviral

### Response

This example describes the quantification and statistical analysis parameters used for the studies described in Example 4.

### Sample Size Calculation

All in vitro experiments were performed in triplicate. A representative experiment of a minimum of three independent experiments is shown in the manuscript, unless indicated otherwise in the Figure legends. Sample size for in vivo experiments was calculated using IBM SPSS statics software (version 28) to achieve a power of 0.8 at p=0.05. A preliminary experiment with the fetal placentas of one dam was used to obtain the mean and standard deviation values to calculate the minimum number of samples needed per experimental group.

### Statistical Analysis

Parametric (two-tailed unpaired t-test with or without Welch's correction and one-way ANOVA with Dunnett's or Tukey's post hoc tests) and non-parametric (Mann-Whitney U and Kruskall Wallis) tests were used where appropriate for statistical analysis of the data using GraphPad Prism 9 software. *P<0.05 was considered to be statistically significant. The investigators were not blinded to the experimental conditions. All data represent the mean±SEM (n=3) of a representative of at least three independent experiments unless otherwise noted in the relevant figure legends.

### Example 4

### Alu SINES of the Mir-498 (46) Cistron Mediate Intrinsic Interferon and Antiviral Response in a Human Placenta

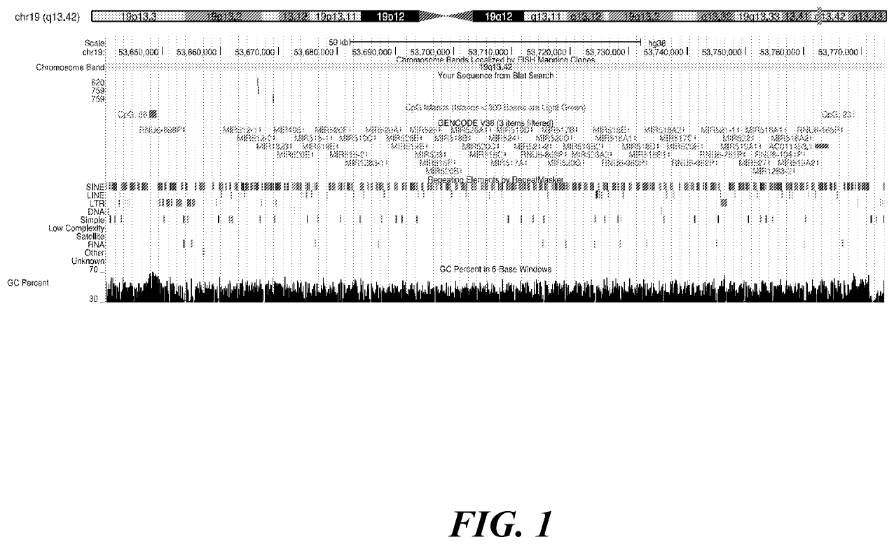

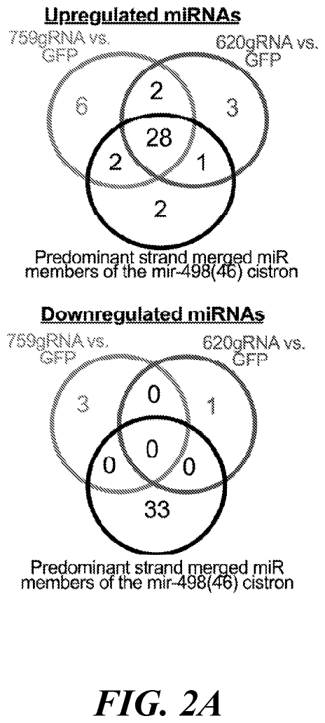

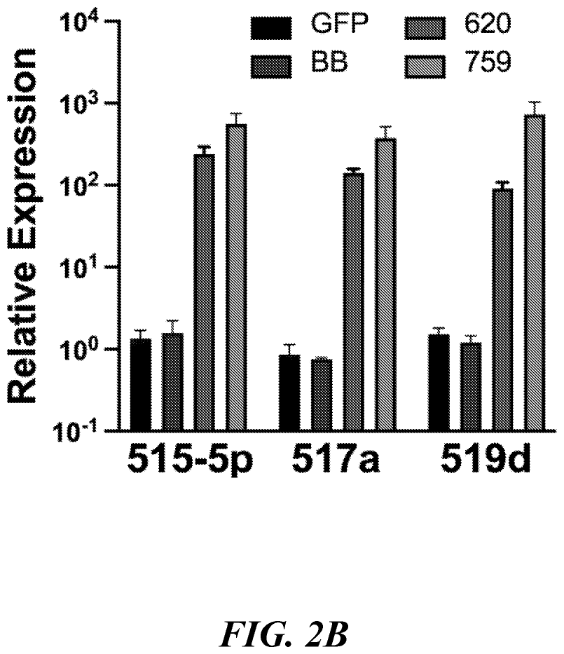

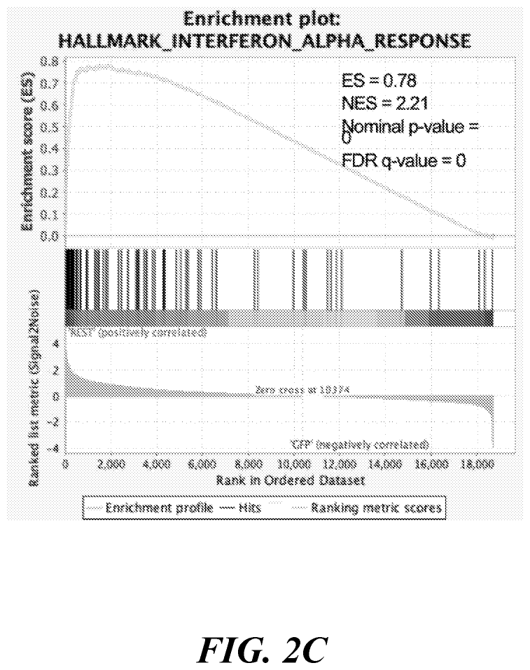

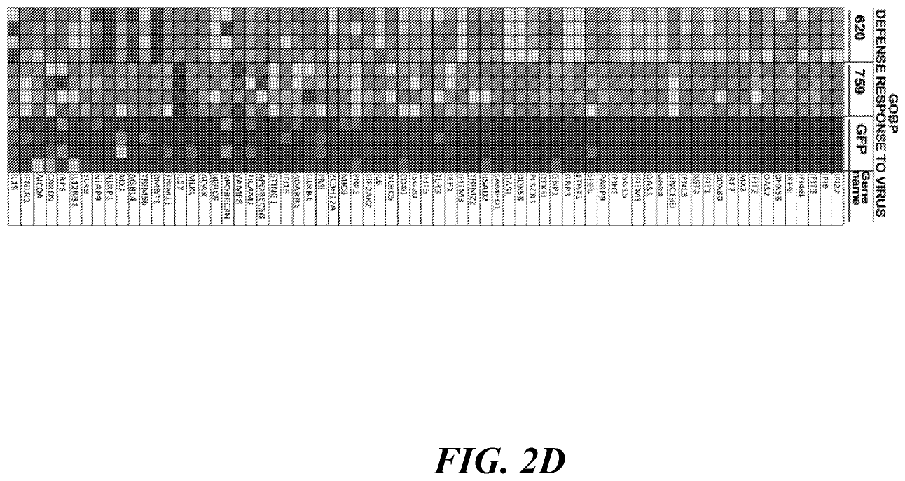

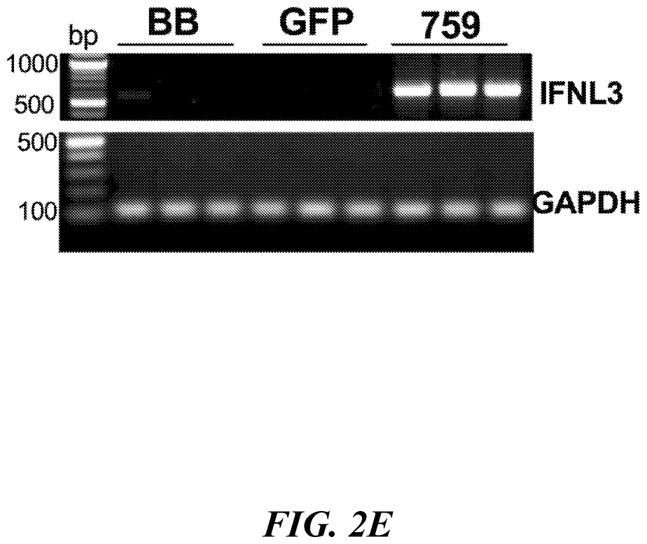

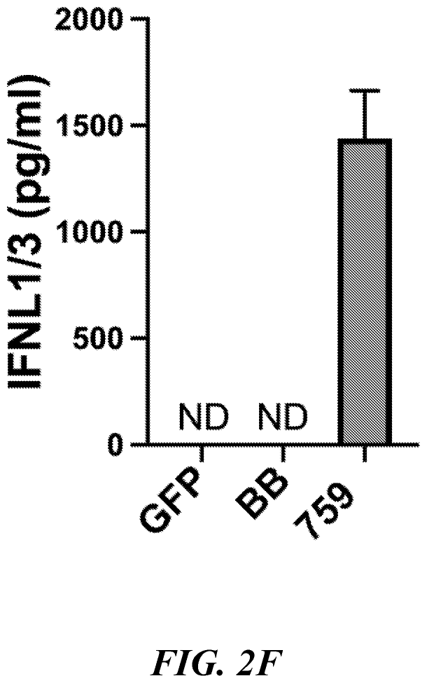

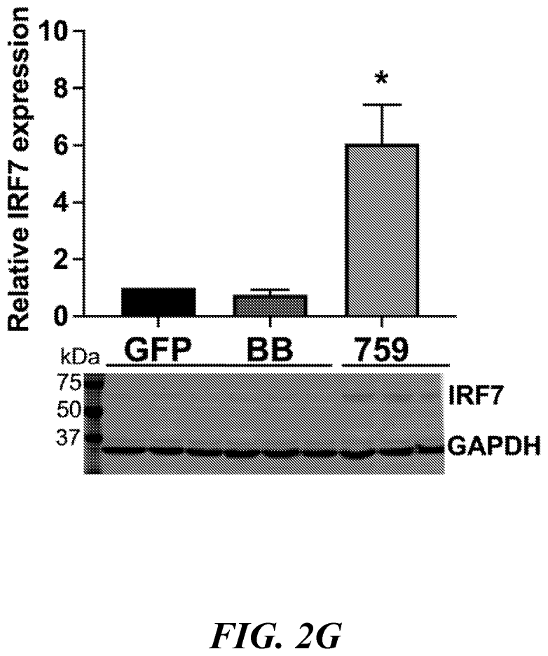

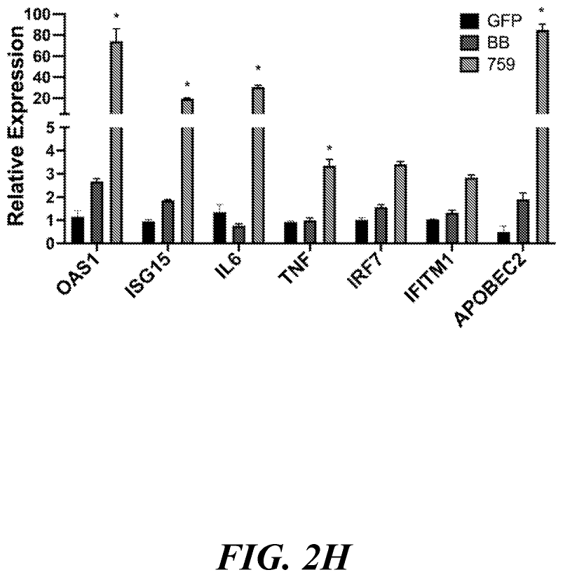

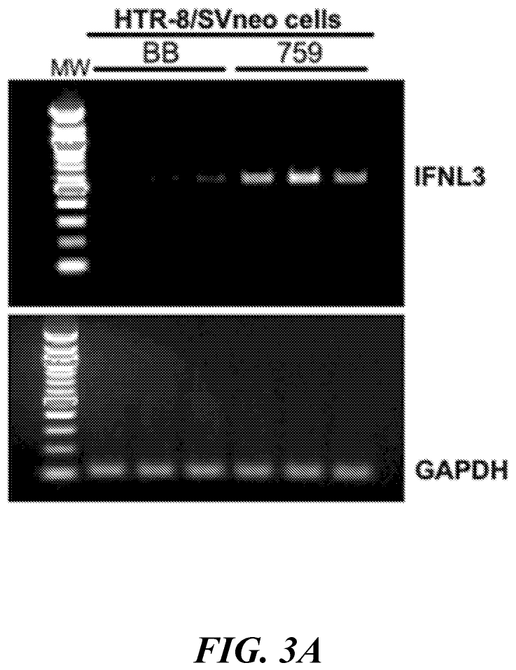

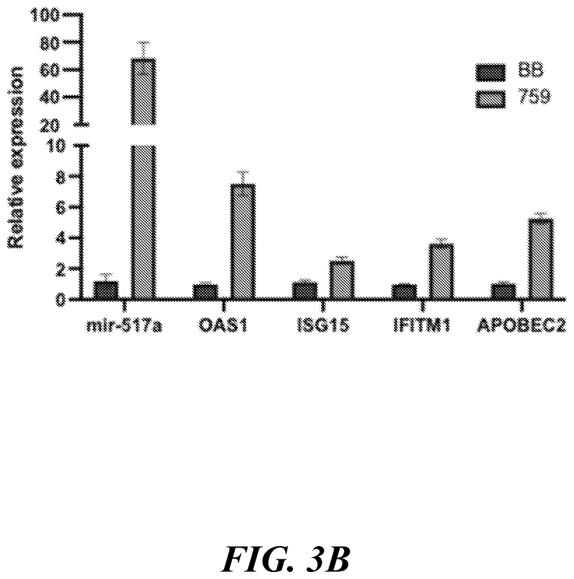

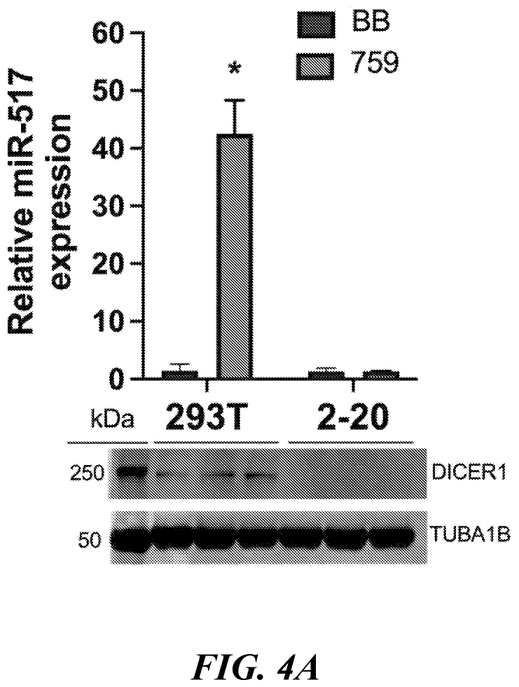

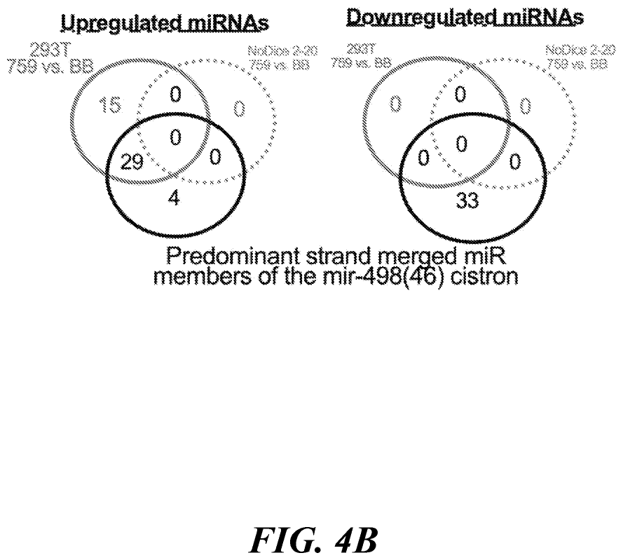

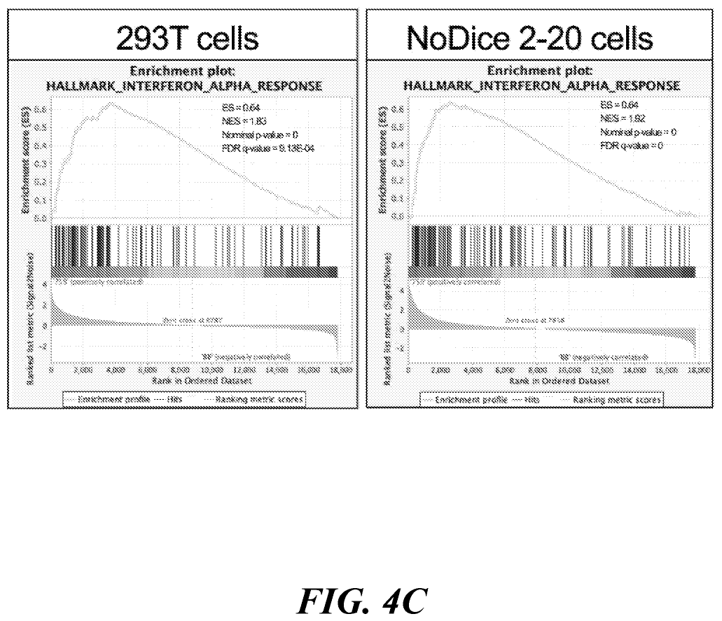

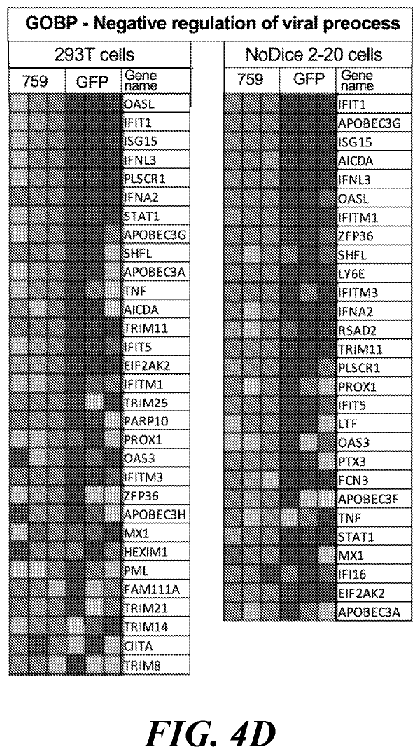

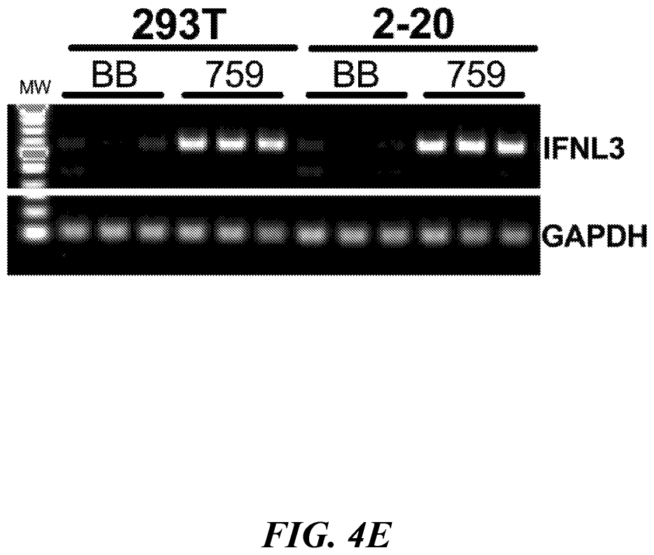

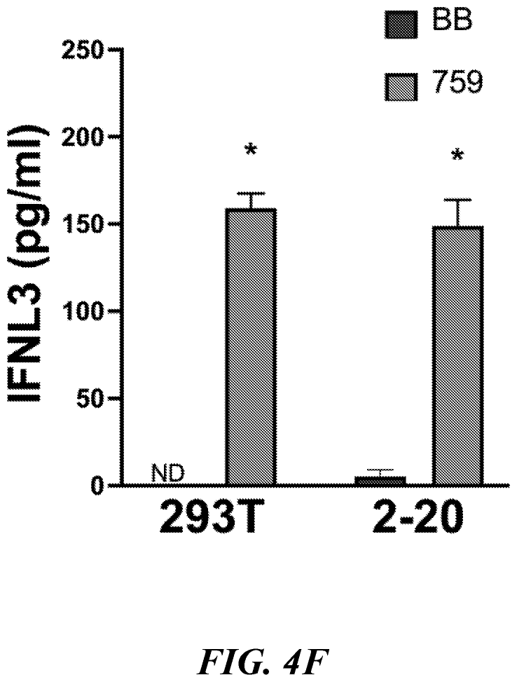

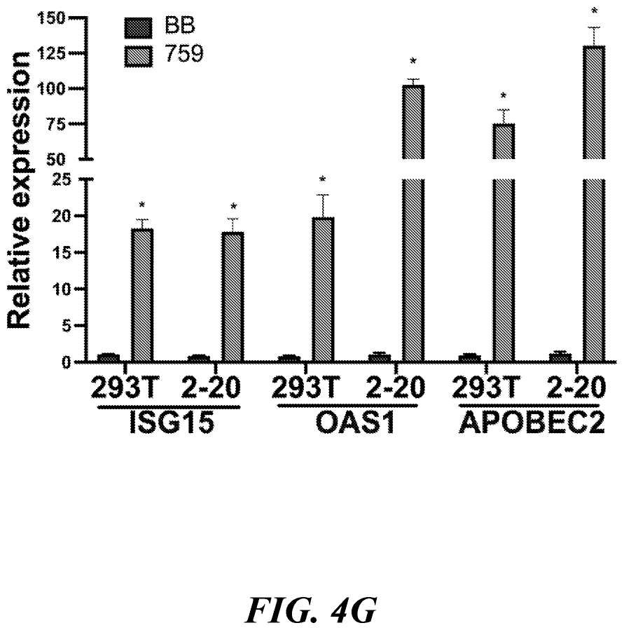

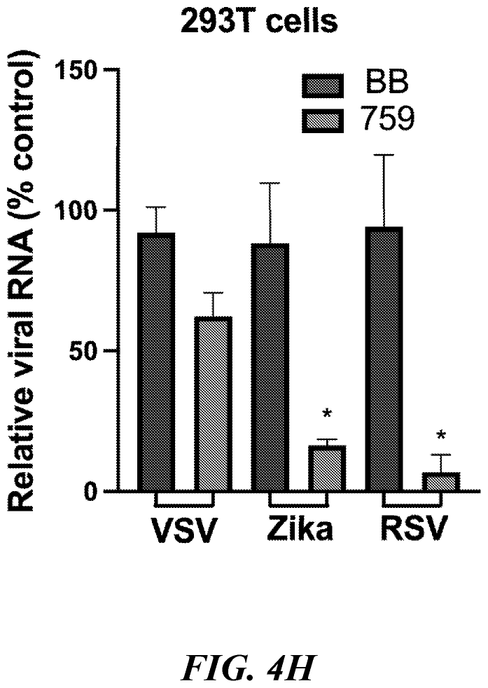

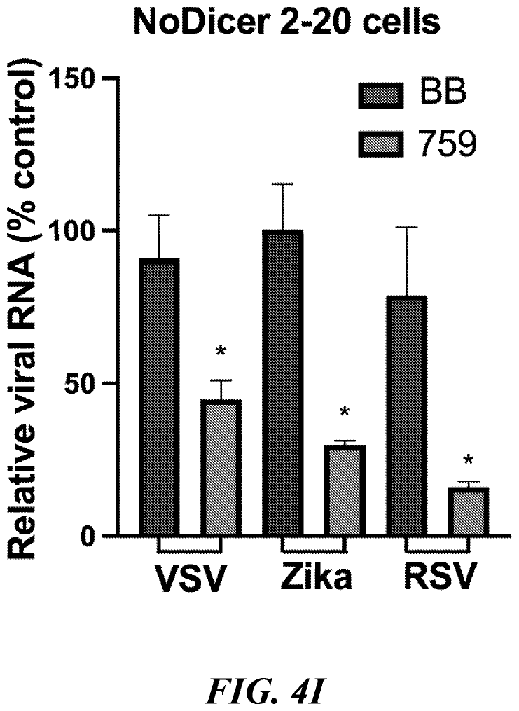

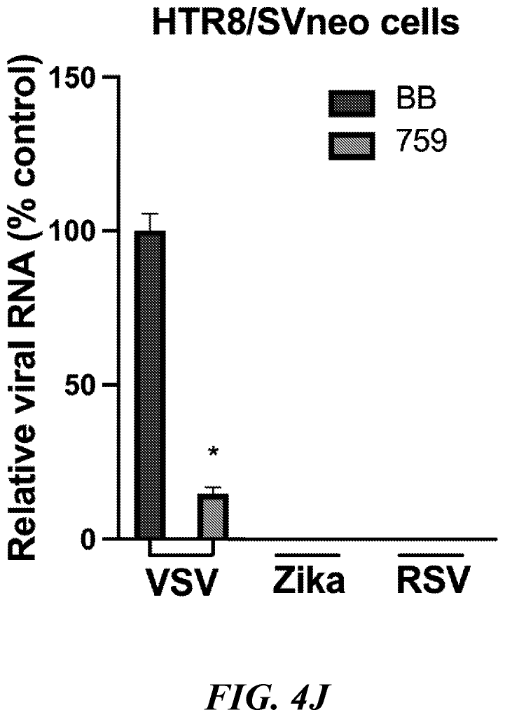

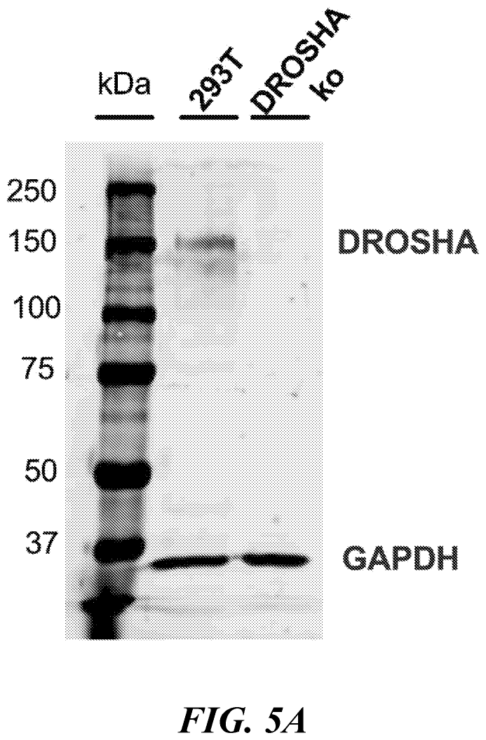

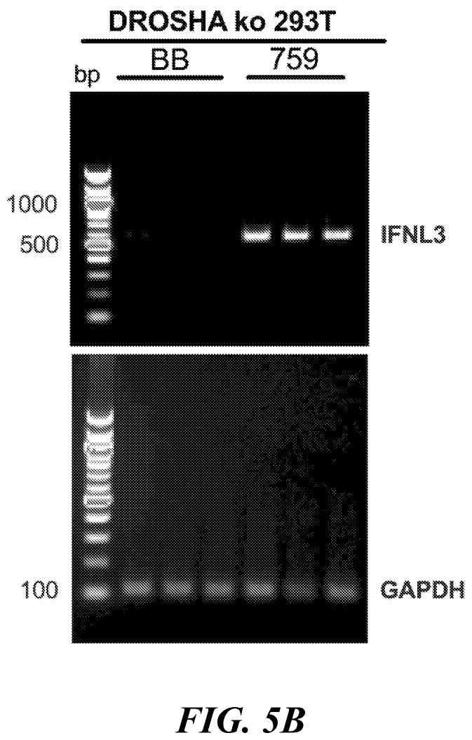

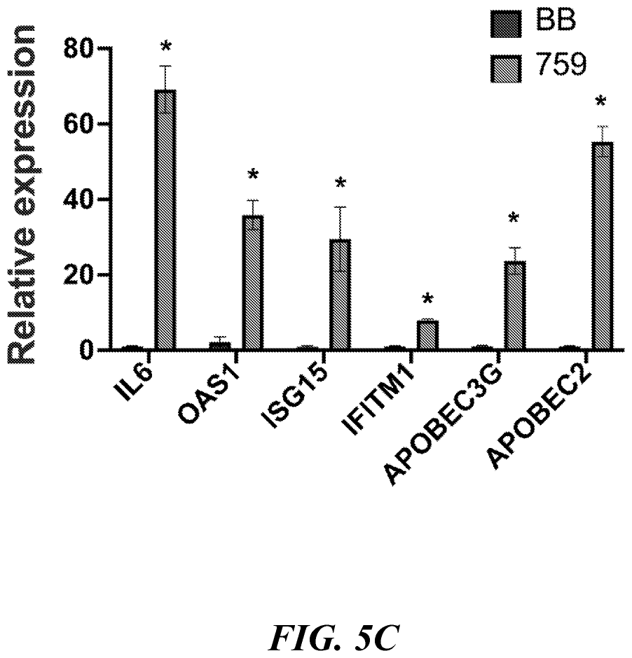

FIG. 1 depicts an activated mir-398 (46) cistron, according to an embodiment of the present disclosure. As such, CRISPR/dCas9-SAM technology and two different sgRNAs, the 620-sgRNA and the 759-sgRNA, were used to transcriptionally activate the cistron that contains 46 pri-miRNAs and 265 Alu SINEs embedded in both the sense and antisense strands. Furthermore, as shown in FIGS. 2A-2H, it was found that transcriptional activation of the mir-498 (46) cistron, even in the absence of viral infection, induces type III interferon (e.g., IFN) and its downstream interferon stimulated genes (e.g., ISGs) and antiviral response genes. FIGS. 3A-3B depict HTR-8/SVneo cells and its relative expression as shown in agarose gel and in graphical interpretation, according to an embodiment of the present disclosure. In this manner, as shown in FIGS. 3A-3B, the HTR-8/SVneo are used to immortalize human extra villous trophoblast that do not express the mir-498 (46) cistron to help establish the relationship between mir-498 (46) cistron and IFNL3 and additional antiviral response genes (e.g., ISGs). As such, the transcriptional activation of the mir-498 (46) cistron induces interferon and antiviral response independently of the mature miRNAs and strongly indicates that the mir-498 (46) cistron produce “other” RNA transcripts, which are responsible for the induction of these effects, as shown in FIGS. 4A-4J. In addition, FIGS. 5A-5C depict a series of images depicting mir-498 (46) cistron activation induced type III IFN, ISGs, and antiviral protection in a microRNA-independent mechanisms, further indicating that the mir-498 (46) cistron induces interferon and antiviral response regardless of the mature miRNAs.

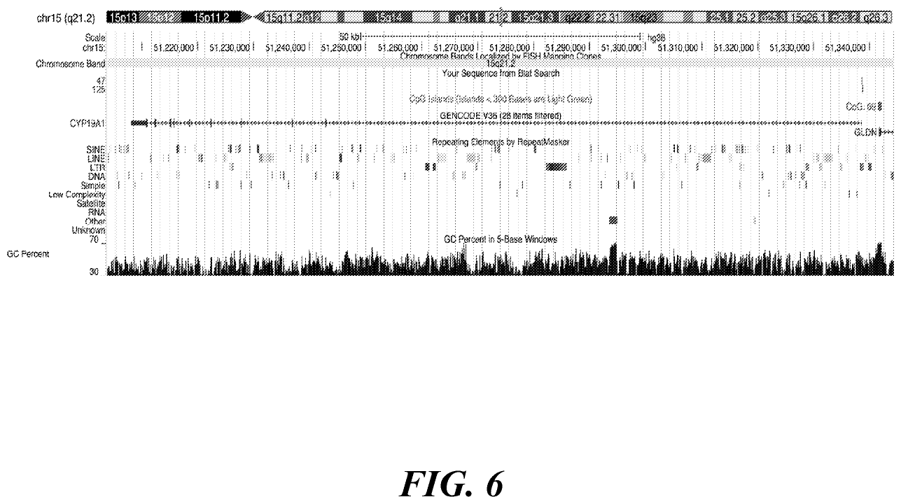

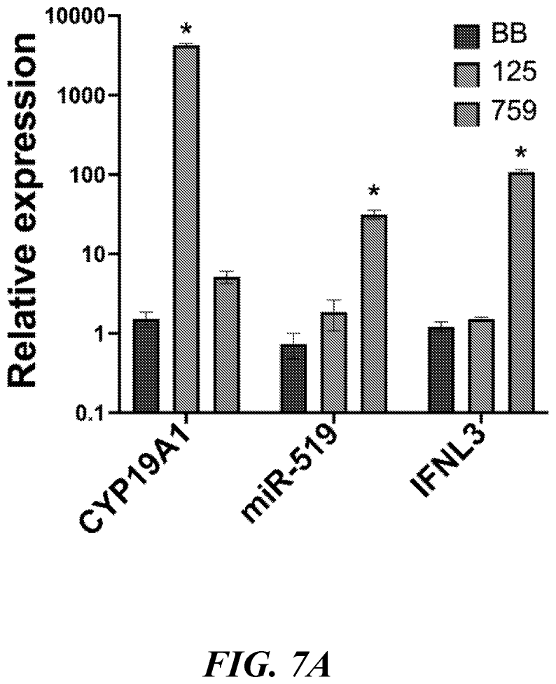

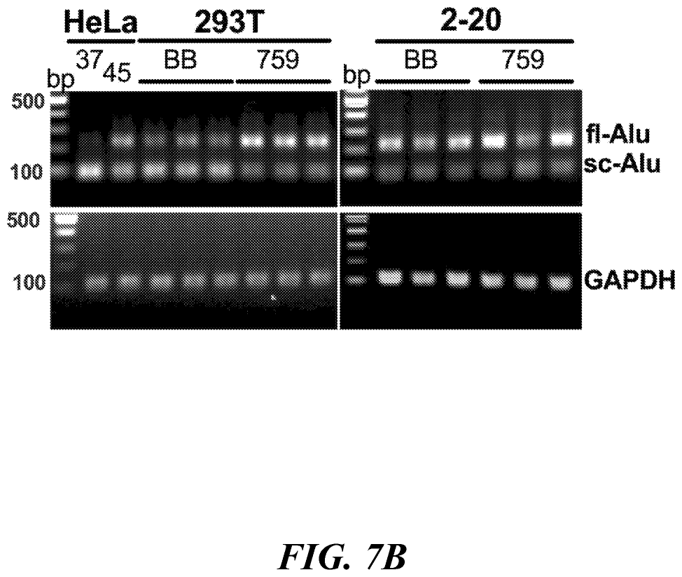

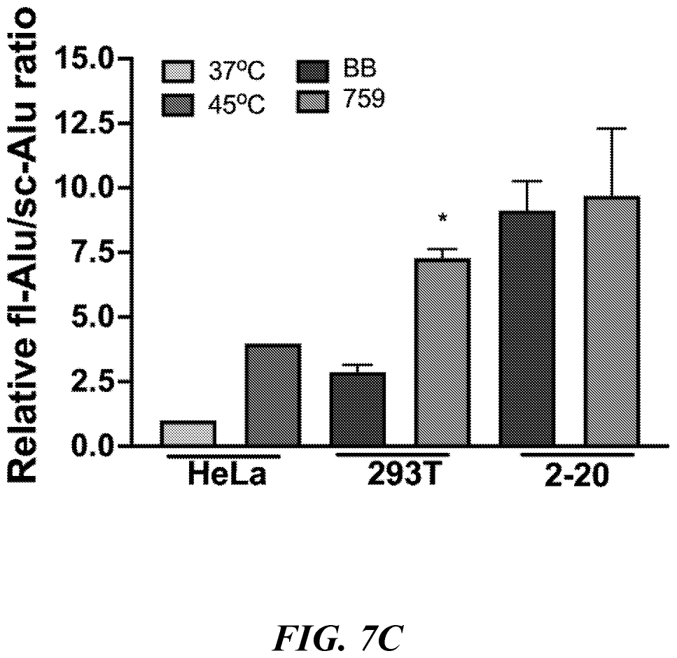

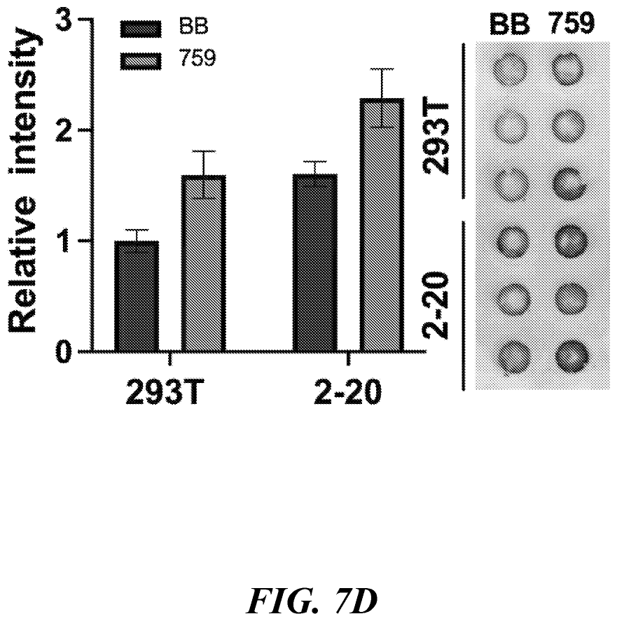

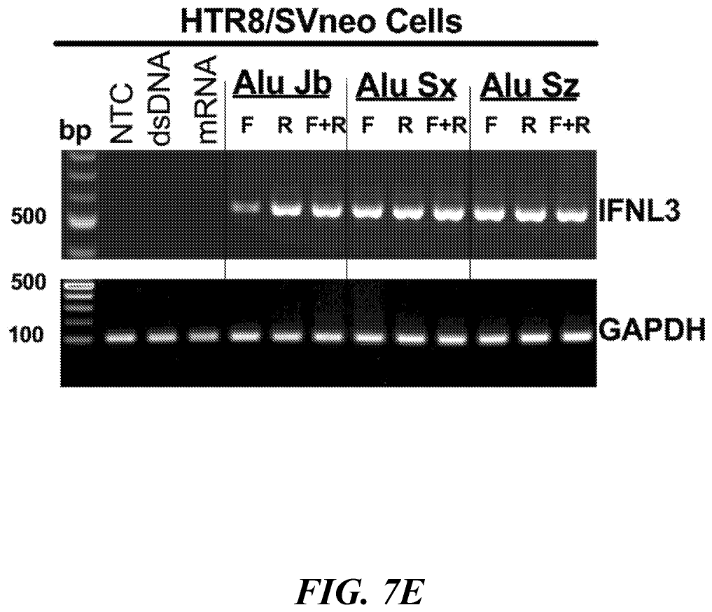

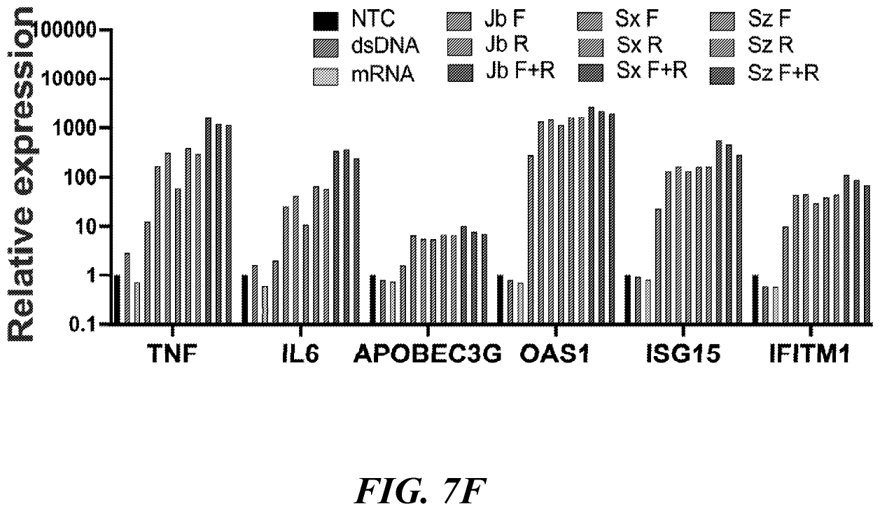

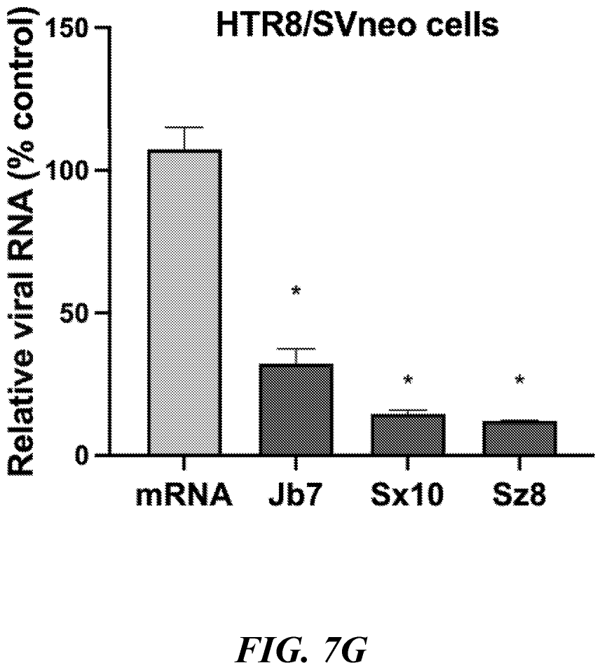

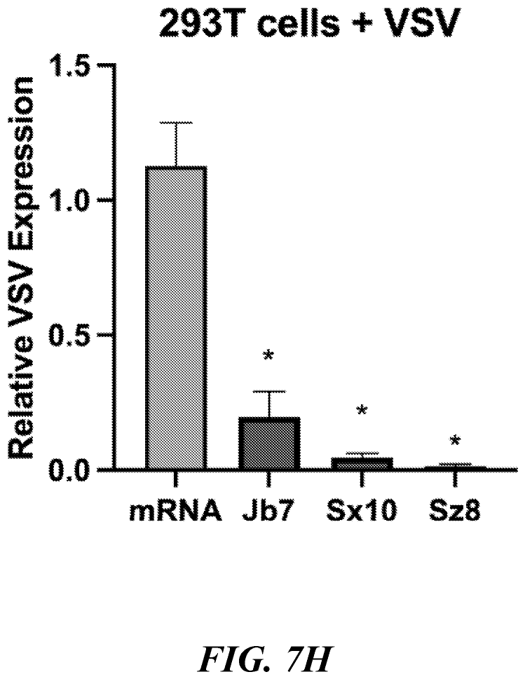

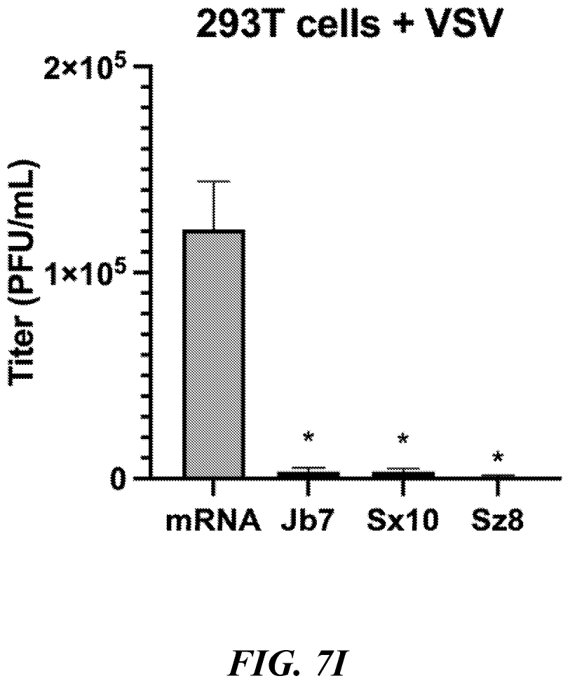

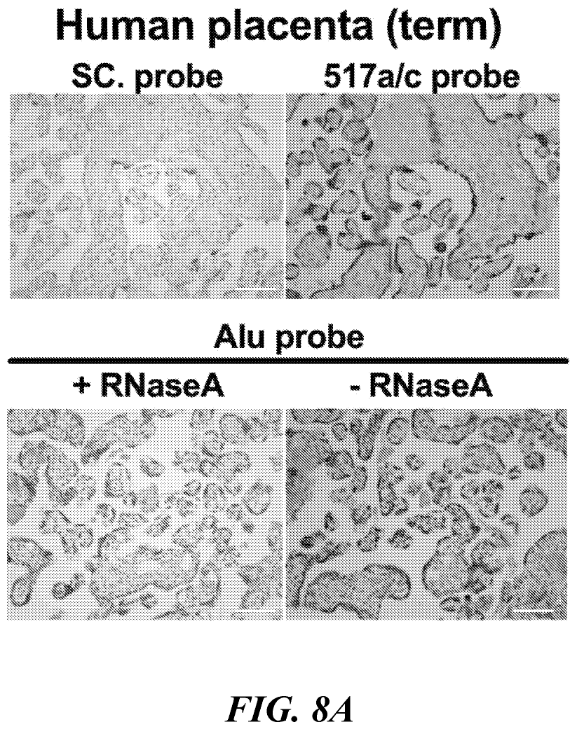

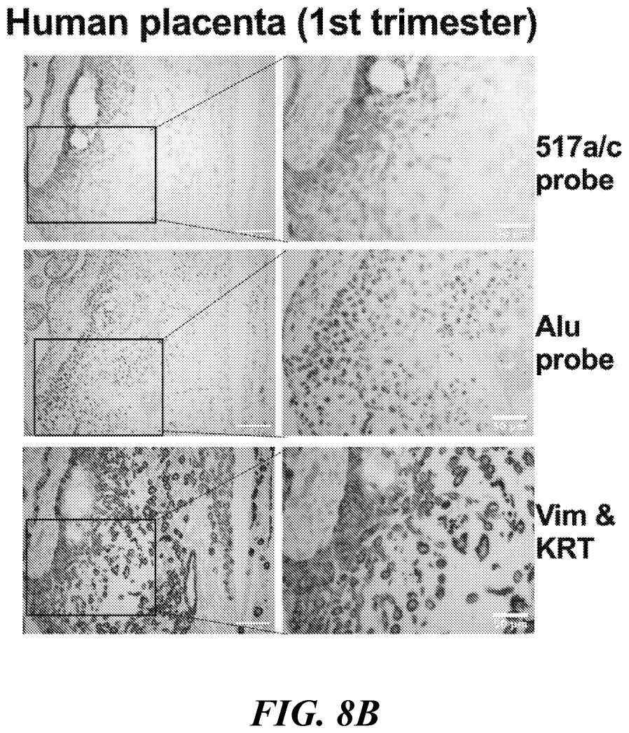

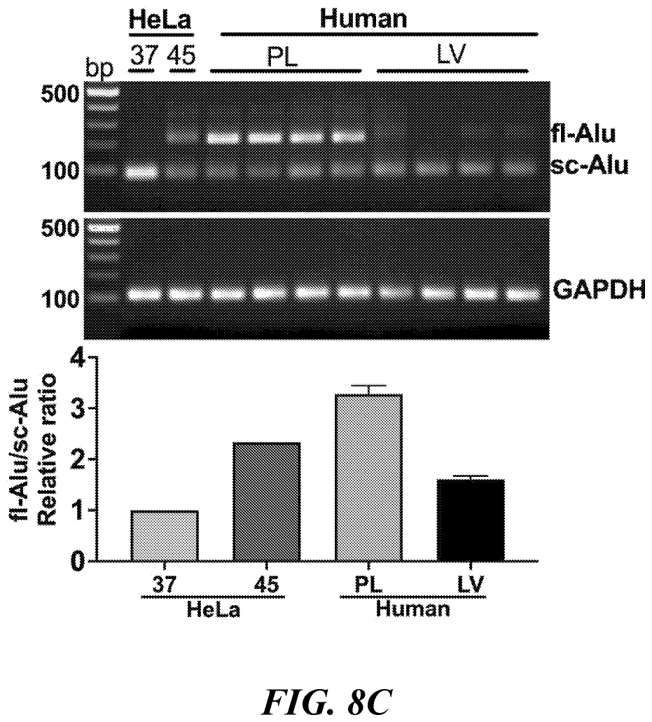

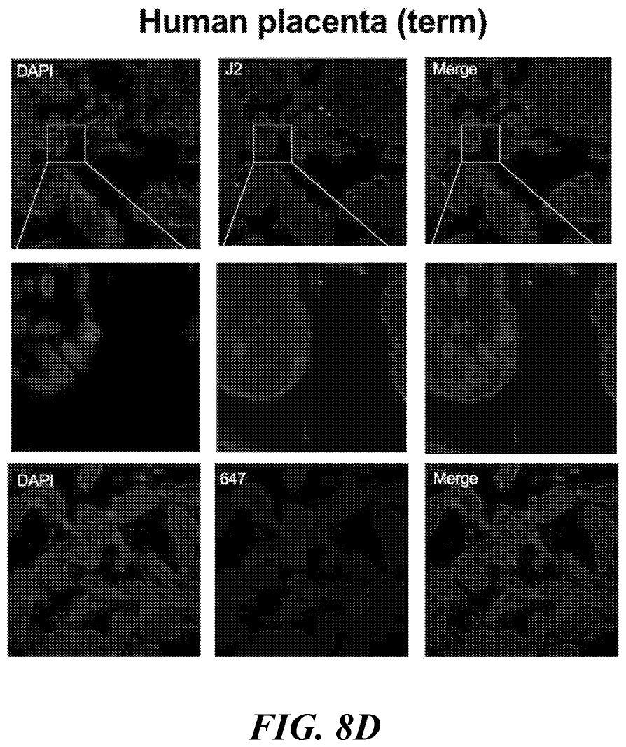

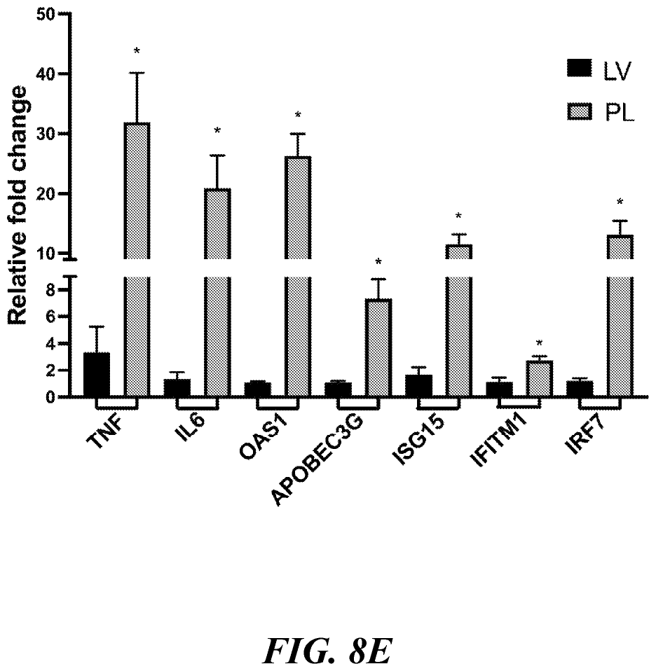

Since about 50% of the mir-498 (46) cistron consist of the highly homologous Alu repeats embedded in both the sense (20%) and antisense (80%) strands, the inventors believe that transcriptional activation of the mir-498 (46) cistron generates Alu double stranded (ds) RNA, which are responsible for the intrinsic induction of type III IFN and the antiviral response. In this manner, as shown in FIG. 6, the activation of the miR-498 (46) cistron increases Alu dsRNA which mediate intrinsic type III IFN production and antiviral protection. As such, as shown in FIGS. 7A-7I, the transcriptional activation of mir-498 (46) cistron substantially increases dsRNA which facilitate and enable the intrinsic type III IFNs production and antiviral protection within the cell. This is further exemplified in FIGS. 8A-8E, which depict the expression of mir-498 (46) miRNAs localized with Alu transcripts and dsRNA in placental villous trophoblast.

Many ISGs are direct targets of interferon regulatory factors (IRFI, IRF3, IRF7), NFκB, or IL-1 signaling. These ISGs can be induced even in the absence of IFN signaling which may explain why ISGs are expressed in the human placenta and EVTs but not IFNs.

To determine the mechanisms of the intrinsic antiviral response of the trophoblast, the molecular mechanisms by which mir-498 (46)-induced Alu transcripts activate IFNs and the antiviral response were identified. As shown in FIG. 1, the regulatory role of the miRNAs of the mir-498 (46) cistron in preventing excessive Alu-mediated IFNs and inflammatory response was determined, in addition to evaluating the protective role of DICER1 and/or ADAR1 in preventing excessive Alu-mediated IFNs and/or inflammatory response. The intrinsic expression of IFNs/ISGs shapes placental antiviral defense.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

### INCORPORATION BY REFERENCE

Liu W M, Chu W M, Choudary P V, Schmid C W. Cell stress and translational inhibitors transiently increase the abundance of mammalian SINE transcripts. Nucleic Acids Res. 1995; 23 (10): 1758-65. doi: 10.1093/nar/23.10.1758. PubMed PMID: 7784180; PMCID: PMC306933.
Szoka et al.; Comparative Properties and Methods of Preparation of Lipid Vesicles (Liposomes); Ann. Rev. Biophys. Bioeng; 9:467, 1980.
Zhang et al.; Cationic lipids and polymers mediated vectors for delivery of siRNA; J Control Release; 123 (1): 1-10, 2007.
Vorhies et al.; Synthetic vs. natural/biodegradable polymers for delivery of shRNA-based cancer therapies; Methods Mol. Biol. 480:11-29; 2009.
Rahbek et al.; Intracellular siRNA and Precursor miRNA Trafficking using Bioresponsive Copolypeptides; J. Gene Med. 10:81-93; 2008.

The sequence listing entitled “ALU SINES OF THE MIR-498 (46) CISTRON MEDIATE INTRINSIC INTERFERON AND ANTIVIRAL RESPONSE IN HUMAN PLACENTA” in XML format, created on May 22, 2023, and being 85,000 bytes in size, is hereby incorporated by reference into this disclosure.

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

It is also to be understood that the following claims are intended to cover all of the generic and specific features of the invention herein described, and all statements of the scope of the invention which, as a matter of language, might be said to fall therebetween.

What is claimed is:

1. A method of predicting susceptibility for infection in a subject, the method comprising:
obtaining an expression level of at least one Alu RNA and at least one miR member in a sample suspected of comprising the infection;
determining a ratio of Alu RNA:miR member within the sample;
obtaining an expression level of Alu RNA and miR members within a control, wherein a control ratio of Alu RNA:miR members is determined;
comparing the Alu RNA:miR member ratio of the sample suspected of comprising the infection to the Alu RNA:miR member control ratio; and
wherein the ratio comprising a higher expression level of the at least one Alu RNA as compared to the expression level of the at least one miR member within the sample suspected of comprising the infection as compared to the control ratio is indicative of a high susceptibility for the infection.

2. The method of claim 1, wherein the infection is VSV, RSV, SARS-CoV2, or Zika virus.

3. The method of claim 1, wherein the at least Alu RNA is configured to express at least one antiviral biological product configured to increase the expression level in the sample suspected of comprising the infection.

4. The method of claim 3, wherein the at least one antiviral biological product is a C19MC, an IFN, an Ifnl2, an Ifnl3, or an ISG.

5. The method of claim 4, wherein the INF comprises a type Ill interferon.

6. The method of claim 3, wherein the at least one Alu RNA generates the at least one antiviral biological product intrinsically.

7. A method of predicting severity of an infection in a subject, the method comprising:
obtaining an expression level of at least one Alu RNA in a sample suspected of comprising the infection;
obtaining a viral burden in a sample suspected of comprising the infection;
determining a ratio of Alu RNA:Viral Burden;
obtaining an expression level of at least one Alu RNA, a viral burden, or both of a control, wherein a control ratio of Alu RNA to viral burn is determined;
comparing Alu RNA:Viral Burden of the sample suspected of comprising the infection to the Alu RNA:Viral Burden control ratio;
wherein the ratio comprising a lower expression level of Alu RNA to a higher viral burden for the sample suspected of comprising the infection as compared to the control ratio is indicative of a high severity of the infection; and
administering at least one additional miR-498 cistron, the Alu RNA portion, in the forward direction, the reverse direction, or both, thereof, or both if a lower expression level of Alu RNA to a higher viral burden is obtained.

8. The method of claim 7, wherein the infection is VSV, RSV, SARS-CoV2, or Zika virus.

9. The method of claim 7, wherein the at least one Alu RNA of the mir-498 cistron is embedded in the sense strands, antisense strands, or both of the mir-498 cistron.

10. The method of claim 9, wherein the at least Alu RNA member of the at least one additional mir-498 cistron is configured to express at least one antiviral biological product, the at least one antiviral biological product being configured to increase an antiviral response within in the sample suspected of comprising the infection.

11. The method of claim 10, wherein the at least one biological product is a C19MC, an IFN, an Ifnl2, an Ifnl3, or an ISG.

12. The method of claim 11, wherein the INF comprises a type III interferon.

13. The method of claim 10, wherein the at least one Alu RNA generates the at least one antiviral biological product intrinsically.

14. A method of treating an infection in a subject, the method comprising:
obtaining a sample suspected of comprising the infection from the subject;
obtaining an expression level of at least one at least one Alu RNA from the sample;
calculating a median expression level using a highest and a lowest value of the expression levels of the at least one at least one Alu RNA, biological product;
determining if the infection of the subject will be sensitive to at least one additional Alu RNA of a miR-498 cistron by comparing the expression level of the sample suspected of comprising the infection to the median expression level;
wherein a low expression level as compared to the median expression level is indicative of the infection;
administering at least one additional miR-498 cistron, the Alu RNA portion, in the forward direction, the reverse direction, or both, thereof, or both to the subject having the low expression level score; and
wherein subsequent to receiving the at least one miR-498, the Alu RNA portion, or both the sample is configured to increase production of at least one antiviral biological product, optimizing an antiviral response of the at least one cell of the subject.

15. The method of claim 14, wherein the infection is VSV, RSV, SARS-CoV2, or Zika virus.

16. The method of claim 14, wherein the at least one Alu RNA of the miR-498 cistron is embedded in the sense strands, antisense strands, or both of the miR-498 cistron.

17. The method of claim 16, further comprising the step of, after administering the at least one addition miR-498 cistron to the subject, generating the at least one Alu RNA, wherein the at least one Alu RNA is configured to generate the at least one antiviral biological product.

18. The method of claim 17, wherein the at least one antiviral biological product is a C19MC, an IFN, an Ifnl2, an Ifnl3, or an ISG.

19. The method of claim 18, wherein the INF comprises a type III interferon.

20. The method of claim 17, wherein the at least one Alu RNA generates the at least one antiviral biological product intrinsically.


SEQUENCE LISTING (78 sequences)

SEQ ID NO: 1 - DNA, 242 bp, Homo sapiens
  source 1..242: mol_type = genomic DNA
aattcgcccttgcaatctccttctcacgggttcaggcaattctcctgcctcagcctccaagtacctgggactacaggcgctggccaccatgcccagctaattttctgtatttttagtagagacggagtttcaccatgttagccaggctggtctcaatttcctgacctcatgatctgcccgccttggcctcccaaagtgctgggactacaagcgtgtgccaccgcacccggaagggcgaattc

SEQ ID NO: 2 - DNA, 243 bp, Homo sapiens
  source 1..243: mol_type = genomic DNA
gaattcgcccttccgggtgcggtggcacacgcttgtagtcccagcactttgggaggccaaggcgggcagatcatgaggtcaggaaattgagaccagcctggctaacatggtgaaactccgtctctactaaaaatacagaaaattagctgggcatggtggccagcgcctgtagtcccaggtacttggaggctgaggcaggagaattgcctgaacccgtgagaaggagattgcaagggcgaattc

SEQ ID NO: 3 - DNA, 243 bp, Homo sapiens
  source 1..243: mol_type = genomic DNA
aattcgcccttgcaatctccttctcacgggttcaagcaattctcctgcctcagcctccaagtacctgggactacaggcgctggccaccatgcccagctaattttctgtatttttggtagagacagagtttcaccatgttagccaggctggtctcaatttcctgacctcatgatctgcccgccttggcctcccaaagtgctgggattacaagcgtgtgccaccgcacccggaagggcgaattcc

SEQ ID NO: 4 - DNA, 243 bp, Homo sapiens
  source 1..243: mol_type = genomic DNA
gaattcgcccttccgggtgcggtggctcacgcttgtaatcccagcactttgggaggccaaggcgggcagatcatgaggtcaggaaattgagaccagcctggctaacatggtgaaactctgtctctaccaaaaatacagaaaattagctgggcatggtggccagcgcctgtagtcccaggtacttggaggctgaggcaggagaattgcttgaacccgtgagaaggagattgcaagggcgaattc

SEQ ID NO: 5 - DNA, 243 bp, Homo sapiens
  source 1..243: mol_type = genomic DNA
gaattcgcccttccgggtgcggtggcacacgcttgtaatcccagcactttgggaggccaaggcgggcagatcatgaggtcaggaaattgagaccagcctggctaacatggtgaaactctgtctccaccaaaaatacagaaaattagctgggcatggtggccagcgcctgtagtcccaggtacttggaggctgaggcaggagaattgcttgaacccgtgagaaggagattgcaagggcgaattc

SEQ ID NO: 6 - DNA, 243 bp, Homo sapiens
  source 1..243: mol_type = genomic DNA
gaattcgcccttgcaatctccttctcacgggttcaagcaattctcctgcctcagcctccaagtacctgggactacaggcgctggccaccatgcccagctaattttctgtatttttggtggagacagagtttcaccatgttagccaggctggtctcaatttcctgacctcatgatctgcccgccttggcctcccaaagtgctgggattacaagcgtgtgccaccgcacccggaagggcgaattc

SEQ ID NO: 7 - DNA, 243 bp, Homo sapiens
  source 1..243: mol_type = genomic DNA
gaattcgcccttgcaatctccttctcacgggttcaagcgattctcctgcctcagcctcccaactaactgggactacaagcgcgcgccaccacgcccagctaatttttgtatttttagtagagacggggcttcaccatgttggccaggatggtctcgatctcctgacctcgtgatccgcccgcctcgacctcccaaagtgctgggattacaagcgtgtgccaccgcacccggaagggcgaattc

SEQ ID NO: 8 - DNA, 243 bp, Homo sapiens
  source 1..243: mol_type = genomic DNA
gaattcgcccttccgggtgcggtggcacacgcttgtaatcccagcactttgggaggtcgaggcgggcggatcacgaggtcaggagatcgagaccatcctggccaacatggtgaagccccgtctctactaaaaatacaaaaattagctgggcgtggtggcgcgcgcttgtagtcccagttagttgggaggctgaggcaggagaatcgcttgaacccgtgagaaggagattgcaagggcgaattc

SEQ ID NO: 9 - DNA, 242 bp, Homo sapiens
  source 1..242: mol_type = genomic DNA
gaattcgcccttccggtgcggtggcacacgcttgtaatcccagcactttgggaggtcgaggcgggcggatcacgaggtcaggagatcgagaccatcctggccaacatggtgaagccccgtctctactaaaaatacaaaaattagctgggcgtggtggcgcgcgcttgtagtcccagttagttgggaggctgaggcaggagaatcgcttgaacccgtgagaaggagattgcaagggcgaattc

SEQ ID NO: 10 - DNA, 242 bp, Homo sapiens
  source 1..242: mol_type = genomic DNA
gaattcgcccttgcaatctccttctcacgggttcaagcgattctcctgcctcagcctcccaactaactgggactacaagcgcgcgccaccacgcccagctaatttttgtatttttagtagagacggggcttcaccatgttggccaggatggtctcgatctcctgacctcgtgatccgcccgcctcgacctcccaaagtgctgggattacaagcgtgtgccaccgcaccggaagggcgaattc

SEQ ID NO: 11 - DNA, 243 bp, Homo sapiens
  source 1..243: mol_type = genomic DNA
gaattcgcccttgcaatctccttctcacgggttcaagcaattctcctgcctcagcctccaagtacctgggactacaggcgctggccaccatgcccagctaattttctgtatttttagtagagacagagtttcaccatgttagccaggctggtctcaatttcctgacctcatgatctgcccgccttggcctcccaaagtgctgggattacaagcgtgtgccaccgcacccggaagggcgaattc

SEQ ID NO: 12 - DNA, 243 bp, Homo sapiens
  source 1..243: mol_type = genomic DNA
gaattcgcccttccgggtgcggtggcacacgcttgtaatcccagcactttgggaggccaaggcgggcagatcatgaggtcaggaaattgagaccagcctggctaacatggtgaaactctgtctctactaaaaatacagaaaattagctgggcatggtggccagcgcctgtagtcccaggtacttggaggctgaggcaggagaattgcttgaacccgtgagaaggagattgcaagggcgaattc

SEQ ID NO: 13 - DNA, 245 bp, Homo sapiens
  source 1..245: mol_type = genomic DNA
gaattcgcccttgcaatctccttctcacgggttcaagctattctcctgcctcagcctcccgagtagctgggactacaggtgcccaccaccatgcccagctacgttttgtatttttagtagagatggggtttcattgtgttggccaggttggtctcaaactcctgacctcaggtgatccgcccgcctcagcctcccaaagtgctgagattacaagcgtgtgccaccgcacccggaagggcgaattc

SEQ ID NO: 14 - DNA, 246 bp, Homo sapiens
  source 1..246: mol_type = genomic DNA
gaattcgcccttccgggtgcggtggcttcacgcttgtaatctcagcactttgggaggctgaggcgggcggatcacctgaggtcaggagtttgagaccaacctggccaacacaatgaaaccccatctctactaaaaatacaaaacgtagctgggcatggtggtgggcacctgtagtcccagctactcgggaggctgaggcaggagaatagcttgaacccgtgagaaggagattgcaagggcgaattc

SEQ ID NO: 15 - DNA, 243 bp, Homo sapiens
  source 1..243: mol_type = genomic DNA
gaattcgcccttgcaatctccttctcacgggttcaagcaattctcctgcctcagcctccaagtacctgggactacaggcgctggccaccatgcccagctaattttctgtatttttagtagagacagagtttcaccatgttagccaggctggtctcaatttcctggcctcatgatctgcccgccttggcctcccaaagtgctgggattacaagcgtgtgccaccgcacccggaagggcgaattc

SEQ ID NO: 16 - DNA, 243 bp, Homo sapiens
  source 1..243: mol_type = genomic DNA
gaattcgcccttccgggtgcggtggcacacgcttgtaatcccagcactttgggaggccaaggcgggcagatcatgaggccaggaaattgagaccagcctggctaacatggtgaaactctgtctctactaaaaatacagaaaattagctgggcatggtggccagcgcctgtagtcccaggtacttggaggctgaggcaggagaattgcttgaacccgtgagaaggagattgcaagggcgaattc

SEQ ID NO: 17 - DNA, 243 bp, Homo sapiens
  source 1..243: mol_type = genomic DNA
gaattcgcccttccgggcgcggtggcacacgcttgtaatcccatcactttgggaggtcgaggcgggcggatcacgatgtcaggagatcgagaccatcctggccaacatggtgaggccccgtctctactaaaaatacaaaaattagctgggggtggtggcgcgcgcttgtagtcccagttagttgggaggctgaggcaggagaatcgcttgaacccgtgagaaggagattgcaagggcgaattc

SEQ ID NO: 18 - DNA, 242 bp, Homo sapiens
  source 1..242: mol_type = genomic DNA
aattcgcccttgcaatctccttctcacgggttcaagcgattctcctgcctcagcctcccaactaactgggactacaagcgcgcgccaccacgcccagctaatttttgtatttttagtagagacggggcttcaccatgttggccaggatggtctcgatctcctgacctcgtgatccgcccgcctcgacctcccaaagtgctgggattacaagcgtgtgccaccgcacccggaagggcgaattc

SEQ ID NO: 19 - DNA, 243 bp, Homo sapiens; identical to SEQ ID NO: 8
  source 1..243: mol_type = genomic DNA

SEQ ID NO: 20 - DNA, 244 bp, Homo sapiens
  source 1..244: mol_type = genomic DNA
gaattcgcccttccgggtgcggtggcacacgcttgtaatcccagcactctgggaggtcgaggcgggcggatcacgaggtcaggagatcgagaccatcctggccggcatggtgaggccccgtctctactaaaaatacaaaaaattagctgggcgtggtggcgcgcgcttgtagtcccagttagttgggaggctgaggcaggagaatcgcttgaacccgtgagaaggagattgcaagggcgaattc

SEQ ID NO: 21 - DNA, 244 bp, Homo sapiens
  source 1..244: mol_type = genomic DNA
gaattcgcccttgcaatctccttctcacgggttcaagcgattctcctgcctcagcctcccaactaactgggactacaagcgcgcgccaccacgcccagctaattttttgtatttttagtagagacggggcctcaccatgccggccaggatggtctcgatctcctgacctcgtgatccgcccgcctcgacctcccagagtgctgggattacaagcgtgtgccaccgcacccggaagggcgaattc

SEQ ID NO: 22 - DNA, 244 bp, Homo sapiens
  source 1..244: mol_type = genomic DNA
gaattcgcccttgcaatctccttctcacgggttcaagcgattctcctgcctcagcctcccaactaactgggactacaagcgcgcgccaccacgcccagctaattttttgtatttttagtagagacggggcttcaccatgttggccaggatggtctcgatctcctgacctcgtgatccgcccacctcggcctcccaaagtgctggggttacaagcgtgtgccaccgcacccggaagggcgaattc

SEQ ID NO: 23 - DNA, 244 bp, Homo sapiens
  source 1..244: mol_type = genomic DNA
gaattcgcccttccgggtgcggtggctcacgcttgtaaccccagcactttgggaggccgaggtgggcggatcacgaggtcaggagatcgagaccatcctggccaacatggtgaagccccgtctctactaaaaatacaaaaaattagctgggcgtggtggcgcgcgcttgtagtcccagttagttgggaggctgaggcaggagaatcgcttgaacccgtgagaaggagattgcaagggcgaattc

SEQ ID NO: 24 - DNA, 243 bp, Homo sapiens; identical to SEQ ID NO: 11
  source 1..243: mol_type = genomic DNA

SEQ ID NO: 25 - DNA, 243 bp, Homo sapiens; identical to SEQ ID NO: 12
  source 1..243: mol_type = genomic DNA

SEQ ID NO: 26 - DNA, 246 bp, Homo sapiens
  source 1..246: mol_type = genomic DNA
gaattcgcccttgcaatctccttctcacgggttcaagcgattctcctgcctcagcctcccaactaactgggactacaagcgcgcgccaccacgcccagctaatttttgtattttttttagtagagacggggcttcaccatgttggccaggatggtctcgatctcctgacctcgtgatccgcccgcctcgacctcccaaagtgctgggattacaagcgtgtgccaccgcacccggaagggcgaattc

SEQ ID NO: 27 - DNA, 246 bp, Homo sapiens
  source 1..246: mol_type = genomic DNA
gaattcgcccttccgggtgcggtggcacacgcttgtaatcccagcactttgggaggtcgaggcgggcggatcacgaggtcaggagatcgagaccatcctggccaacatggtgaagccccgtctctactaaaaaaaatacaaaaattagctgggcgtggtggcgcgcgcttgtagtcccagttagttgggaggctgaggcaggagaatcgcttgaacccgtgagaaggagattgcaagggcgaattc

SEQ ID NO: 28 - DNA, 243 bp, Homo sapiens; identical to SEQ ID NO: 7
  source 1..243: mol_type = genomic DNA

SEQ ID NO: 29 - DNA, 244 bp, Homo sapiens
  source 1..244: mol_type = genomic DNA
gaattcgcccttccgggtgcggtggccacacgcttgtaatcccagcactttgggaggtcgaggcgggcggatcacgaggtcaggagatcgagaccatcctggccaacatggtgaagccccgtctctactaaaaatacaaaaattagctgggcgtggtggcgcgcgcttgtagtcccagttagttgggaggctgaggcaggagaatcgcttgaacccgtgagaaggagattgcaagggcgaattc

SEQ ID NO: 30 - DNA, 243 bp, Homo sapiens; identical to SEQ ID NO: 8
  source 1..243: mol_type = genomic DNA

SEQ ID NO: 31 - DNA, 243 bp, Homo sapiens; identical to SEQ ID NO: 7
  source 1..243: mol_type = genomic DNA

SEQ ID NO: 32 - DNA, 244 bp, Homo sapiens
  source 1..244: mol_type = genomic DNA
gaattcgcccttgcaatctccttctcacgggttcaagagattctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcgacctcccaaagtgctgggattacaagcgtgggccaccgcacccggaagggcgaattc

SEQ ID NO: 33 - DNA, 244 bp, Homo sapiens
  source 1..244: mol_type = genomic DNA
gaattcgcccttccgggtgcggtggcccacgcttgtaatcccagcactttgggaggtcgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 34 - DNA, 243 bp, Homo sapiens; identical to SEQ ID NO: 11
  source 1..243: mol_type = genomic DNA

SEQ ID NO: 35 - DNA, 243 bp, Homo sapiens; identical to SEQ ID NO: 12
  source 1..243: mol_type = genomic DNA

SEQ ID NO: 36 - DNA, 243 bp, Homo sapiens; identical to SEQ ID NO: 7
  source 1..243: mol_type = genomic DNA

SEQ ID NO: 37 - DNA, 243 bp, Homo sapiens; identical to SEQ ID NO: 8
  source 1..243: mol_type = genomic DNA

SEQ ID NO: 38 - DNA, 243 bp, Homo sapiens; identical to SEQ ID NO: 7
  source 1..243: mol_type = genomic DNA

SEQ ID NO: 39 - DNA, 243 bp, Homo sapiens; identical to SEQ ID NO: 8
  source 1..243: mol_type = genomic DNA

SEQ ID NO: 40 - DNA, 243 bp, Homo sapiens
  source 1..243: mol_type = genomic DNA
gaattcgcccttgcaatctccttctcacgggttcaagcgattctcctgcctcagcctcccaactaaccgggaccacaagcgcgcgccaccacgcccagccaatttttgtatttttagtagagacggggcttcaccatgttggccaggatggtctcgatctcctgacctcgtgatccgcccgcctcgacctcccaaagcgctgggattacaagcgtgtgccaccgcacccggaagggcgaattc

SEQ ID NO: 41 - DNA, 243 bp, Homo sapiens
  source 1..243: mol_type = genomic DNA
gaattcgcccttccgggtgcggtggcacacgcttgtaatcccagcgctttgggaggtcgaggcgggcggatcacgaggtcaggagatcgagaccatcctggccaacatggtgaagccccgtctctactaaaaatacaaaaattggctgggcgtggtggcgcgcgcttgtggtcccggttagttgggaggctgaggcaggagaatcgcttgaacccgtgagaaggagattgcaagggcgaattc

SEQ ID NO: 42 - DNA, 240 bp, Homo sapiens
  source 1..240: mol_type = genomic DNA
gaattcgcccttgtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 43 - DNA, 243 bp, Homo sapiens
  source 1..243: mol_type = genomic DNA
gaattcgcccttgcatctccttctcacgggttcaagagattctcctgctgccgcctcccacgtagctgagactacaggtgtgtactaccacgcctagctatattttgcatttttagtagagaggaggttttgtcttgatggcggggctgctcttgaactctgacctgaggtgatccacccgtctcgacctcccaaagtgctgggattacaagcgtgtgccaccgcacccggaagggcgaattc

SEQ ID NO: 44 - DNA, 244 bp, Homo sapiens
  source 1..244: mol_type = genomic DNA
gaattcgcccttccgggtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtagtacacacctgtagtctcagctacgtgggaggcggcagcaggagaatctcttgaacccgtgagaaggagattgcaagggcgaattc

SEQ ID NO: 45 - DNA, 241 bp, Homo sapiens
  source 1..241: mol_type = genomic DNA
gaattcgcccttgcatctccttctcacgggttcaagagattctcctgctgccgcctcccacgtagtctgagactacaggtgtgtaccaccacgcctagctatattttgcatttttagtagagaggaggttttatcttgatggcggggctgctcttgaactctgacctgaggtgatccacccgtctcgacctcccaaagtgctgggattacaagcgtgtgccaccgcaccaagggcgaattc

SEQ ID NO: 46 - DNA, 241 bp, Homo sapiens
  source 1..241: mol_type = genomic DNA
gaattcgcccttggtgcggtggcacacgcttgtaatcccagcactttgggaggtcgagacgggtggatcacctcaggtcagagttcaagagcagccccgccatcaagataaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 47 - DNA, 258 bp, Homo sapiens
  source 1..258: mol_type = genomic DNA
gattcccttgcaatctccttctcacgggttcaagagattctcctgctgccgcctcccacgcagctgagactacaggtgtgtaccaccacgcctagctatattttgcatttttagtagagaggaggttttgtcttgatggcggggctgctcttgaactctgacctgaggtgatccgcccgcctcgacctcccaagctgctgttattacgatcccgtgccaccgccatagtgaggacgactccgcctcaaccttcaattc

SEQ ID NO: 48 - DNA, 244 bp, Homo sapiens
  source 1..244: mol_type = genomic DNA
gaattcgcccttccgggtgcggtggcacacgcttgtaatcccagcactttgggaggtcgagacgggcggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 49 - DNA, 241 bp, Homo sapiens
  source 1..241: mol_type = genomic DNA
gaattcgcccttgggtgcggtggcacacgcttgtaatcccagcactttgggaggccaaggcgggcagatcatgaggtcaggaaattgagaccagcctggctaacatggtgaaactctgtctctactaaaaatacagaaaattagctgggcatggtggccagcgcctgtagtcccaggtacttggaggctgaggcaggagaattgcttgaacccgtgagaaggagattgcaagggcgaattc

SEQ ID NO: 50 - DNA, 109 bp, Homo sapiens
  source 1..109: mol_type = genomic DNA
gaattcgcccttgcatctccttctcacgggttcaagcgattctcctgcctcagcctcctgaatagctgggaccacaagcgtgtgccaccgcacccggaagggcgaattc

SEQ ID NO: 51 - DNA, 111 bp, Homo sapiens
  source 1..111: mol_type = genomic DNA
gaattcgcccttccgggtgcggtggcacacgcttgtggtcccagctattcaggaggctgaggccaggagaatcgcttgaacccgtgagaaggagattgcaagggcgaattc

SEQ ID NO: 52 - DNA, 242 bp, Homo sapiens
  source 1..242: mol_type = genomic DNA
gaattcgcccttgcatctccttctcacgggttcaagcgattctcctgcctcagcctcccaactaactgggactacaagcgcgcgccaccacgcccagctaatttttgtatttttagtagagacggggcttcaccatgttggccaggatggtctcgatctcctgacctcgtgatccgcccgcctcgacctcccaaagtgctgggattacaagcgtgtgccaccgcacccggaagggcgaattc

SEQ ID NO: 53 - DNA, 244 bp, Homo sapiens
  source 1..244: mol_type = genomic DNA
gaattcgcccttccgggtgcggtggcacacgcttgtaatcccagcactttgggaggtcgaggccgggcggatcacgaggtcaggagatcgagaccatcctggccaacatggtgaagccccgtctctactaaaaatacaaaaattagctgggcgtggtggcgcgcgcttgtagtcccagttagttgggaggctgaggcaggagaatcgcttgaacccgtgagaaggagattgcaagggcgaattc

SEQ ID NO: 54 - DNA, 243 bp, Homo sapiens
  source 1..243: mol_type = genomic DNA
gaattcgcccttgcatctccttctcacgggttcaagagatcctcctgctgccgcctcccacgtagctgagactacaggtgtgtaccaccacgcctagctatattttgcatttttagtagagaggaggttttgtcttgatggcggggctgctcttggactctgacctgaggtgatccacccgtctcgacctcccaaagtgctgggattacaagcgtgtgccaccgcacccggaagggcgaattc

SEQ ID NO: 55 - DNA, 244 bp, Homo sapiens
  source 1..244: mol_type = genomic DNA
gaattcgcccttccgggtgcggtggcacacgcttgtaatcccagcactttgggaggtcgagacgggtggatcacctcaggtcagagtccaagagcagccccgccatcaagacaaaacctcctctctactaaaaatgcaaaatatagctaggcgtggtggtacacacctgtagtctcagctacgtgggaggcggcagcaggaggatctcttgaacccgtgagaaggagattgcaagggcgaattc

SEQ ID NO: 56 - DNA, 241 bp, Homo sapiens
  source 1..241: mol_type = genomic DNA
gaattcgcccttcgtgcggtggcacacgcttgtaatcccagcactttgggaggtcgagacgggtggatcgcctcaggtcagagttcaagagcagccccgccatcaagacaaaacctcctctctactaaaaatgcaaaatatagctaggcgtggtggttcacacctgtagtctcagctacgtgggaggcggcagcaggagaatctcttgaacccgtgagaaggagattgcaagggcgaattc

SEQ ID NO: 57 - DNA, 241 bp, Homo sapiens
  source 1..241: mol_type = genomic DNA
gaattcgcccttgcatctccttctcacgggttcaagagattctcctgctgccgcctcccacgtagtctgagactacaggtgtgaaccaccacgcctagctatattttgcatttttagtagagaggaggttttgtcttgatggcggggctgctcttgaactctgacctgaggcgatccacccgtctcgacctcccaaagtgctgggattacaagcgtgtgccaccgcacgaagggcgaattc

SEQ ID NO: 58 - DNA, 244 bp, Homo sapiens
  source 1..244: mol_type = genomic DNA
gaattcgcccttgggtgcggtggcacacgcttgtaataccagcactttgggaggccaaggtgggcggatcacctgaggtcaggagttcaagaccagcctggccaatatggtgaaagtccgtctctactaaaattacaaaaattagccgggcgtggtggcagatcccttgtagtcccagctactcgggaggctgaggcaggagaatagcttgaacccgtgagaaggagattgcaagggcgaattc

SEQ ID NO: 59 - DNA, 241 bp, Homo sapiens
  source 1..241: mol_type = genomic DNA
aattcgcccttgcatctccttctcacgggttcaagctattctcctgcctcagcctcccgagtagctgggactacaagggatctgccaccacgcccggctaatttttgtaattttagtagagacggactttcaccatattggccaggctggtcttgaactcctgacctcaggtgatccgcccaccttggcctcccaaagtgctggtattacaagcgtgtgccaccgcaccaagggcgaattc

SEQ ID NO: 60 - DNA, 244 bp, Homo sapiens
  source 1..244: mol_type = genomic DNA
gaattcgcccttgcaatctccttctcacgggttcaagagactctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcgacctcccaaagtgctgggattacaagcgtgtgccaccgcacccggaagggcgaattc

SEQ ID NO: 61 - DNA, 244 bp, Homo sapiens
  source 1..244: mol_type = genomic DNA
gaattcgcccttccgggtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagagtctcttgaacccgtgagaaggagattgcaagggcgaattc

SEQ ID NO: 62 - DNA, 244 bp, Homo sapiens
  source 1..244: mol_type = genomic DNA
gaattcgcccttccgggtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 63 - DNA, 241 bp, Homo sapiens
  source 1..241: mol_type = genomic DNA
attcgcccttgcatctccttctcacgggttcaagagattctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcgacctcccaaagtgctgggattacaagcgtgtgccaccgcacccggaagggcgaattc

SEQ ID NO: 64 - DNA, 110 bp, Homo sapiens
  source 1..110: mol_type = genomic DNA
gaattcgcccttgcaatctccttctcacgggttcaagcgattctcctgcctcagcctcctgagtagctgggattacaagcgtgtgccaccgcacccggaagggcgaattc

SEQ ID NO: 65 - DNA, 244 bp, Homo sapiens
  source 1..244: mol_type = genomic DNA
gaattcgcccttgcaatctccttctcacgggttcaagagattctcctgctgccgcctcccacgtagctgagactacaggtgtgtaccaccacgcctagctatattttgcatttttagtagagaggaggttttgtcttgatggcggggctgctctcgaactctgacctgaggtgatccacccgtctcgacctcccaaagtgctgggattacaagcgtgtgccaccgcacccggaagggcgaattc

SEQ ID NO: 66 - DNA, 244 bp, Homo sapiens
  source 1..244: mol_type = genomic DNA
gaattcgcccttccgggtgcggtggcacacgcttgtaatcccagcactttgggaggtcgagacgggtggatcacctcaggtcagagttcgagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 67 - DNA, 244 bp, Homo sapiens
  source 1..244: mol_type = genomic DNA
gaattcgcccttgcaatctccttctcacgggttcaagagattctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcggcctcccaaagtgctgggattacaagcgtgtgccaccgcacccggaagggcgaattc

SEQ ID NO: 68 - DNA, 242 bp, Homo sapiens
  source 1..242: mol_type = genomic DNA
attcgcccttccgggtgcggtggcacacgcttgtaatcccagcactttgggaggccgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 69 - DNA, 239 bp, Homo sapiens
  source 1..239: mol_type = genomic DNA
gaattcgcccttgggtgcggtggcacacgcttgtaatcccagcactttgggaggccgaggtgagcggatcatgaggtcaggagtttgagaccagcctggccaacacagtgaaaacccgtctactaaaagtacaaaaattagctgggcatggtggcaggcgcctgtaataccagctactcgggagactgaggcaggagaatcgcttgaacccgtgagaaggagattgcaagggcgaattc

SEQ ID NO: 70 - DNA, 236 bp, Homo sapiens
  source 1..236: mol_type = genomic DNA
attcgcccttgcaatctccttctcacgggttcaagcgattctcctgcctcagtctcccgagtagctggtattacaggcgcctgccaccatgcccagctaatttttgtacttttagtagacgggttttcactgtgttggccaggctggtctcaaactcctgacctcatgatccgctcacctcggcctcccaaagtgctgggattacaagcgtgtgccaccgcaccaagggcgaattc

SEQ ID NO: 71 - DNA, 240 bp, Homo sapiens
  source 1..240: mol_type = genomic DNA
gaattcgcccttgcatctccttctcacgggttcaagcgattctcctgcctcagcctcccaactaactgggactacaagcgcgcgccaccacgcccagctaatttttgtatttttagtagagacggggcttcaccatgttggccaggatggtctcgatctcctgacctcgtgatccgcccgcctcgacctcccaaagtgctgggattacaagcgtgtgccaccgcacccaagggcgaattc

SEQ ID NO: 72 - DNA, 239 bp, Homo sapiens
  source 1..239: mol_type = genomic DNA
attcgcccttgggtgcggtggcacacgcttgtaatcccagcactttgggaggtcgaggcgggcggatcacgaggtcaggagatcgagaccatcctggccaacatggtgaagccccgtctctactaaaaatacaaaaattagctgggcgtggtggcgcgcgcttgtagtcccagttagttgggaggctgaggcaggagaatcgcttgaacccgtgagaaggagattgcaagggcgaattc

SEQ ID NO: 73 - DNA, 241 bp, Homo sapiens
  source 1..241: mol_type = genomic DNA
gaattcgcccttgcaatctccttctcacgggttcaagagattctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcgacctcccaaagtgctgggattacaagcgtgtgccaccgcaccaagggcgaattc

SEQ ID NO: 74 - DNA, 242 bp, Homo sapiens
  source 1..242: mol_type = genomic DNA
gaattcgcccttgggtgcggtggcacacgcttgtaatcccagcactttgggaggtcgagacgggtggatcacctcaggtcagagttcaagagcagccccgccatcaagacaaaacctcctctctactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 75 - DNA, 240 bp, Homo sapiens
  source 1..240: mol_type = genomic DNA
gaattcgcccttgcatctccttctcacgggttcaagagattctcctgctgccgcctcccacgtagctgagactacaggtgtgtaccaccacgcctagctatattttgcatttttagtggagaggaggttttgtcttgatggcggggctgctcttgaactctgacctgaggtgatccacccgtctcgacctcccaaagtgctgggattacaagcgtgtgccaccgcaccaagggcgaattc

SEQ ID NO: 76 - DNA, 242 bp, Homo sapiens
  source 1..242: mol_type = genomic DNA
gaattcgcccttgggtgcggtggcacacgcttgtaatcccagcactttgggaggtcgagacgggtggatcacctcaggtcagagttcaagagcagccccgccatcaagacaaaacctcctctccactaaaaatgcaaaatatagctaggcgtggtggtacacacctgtagtctcagctacgtgggaggcggcagcaggagaatctcttgaacccgtgagaaggagattgcaagggcgaattc

SEQ ID NO: 77 - DNA, 242 bp, Homo sapiens; identical to SEQ ID NO: 74
  source 1..242: mol_type = genomic DNA

SEQ ID NO: 78 - DNA, 240 bp, Homo sapiens
  source 1..240: mol_type = genomic DNA
attcgcccttgcaatctccttctcacgggttcaagagattctcctgctgccgcctcccacgtagctgagactacaggtgtgtaccaccacgcctagctatattttgcatttttagtagagaggaggttttgtcttgatggcggggctgctcttgaactctgacctgaggtgatccacccgtctcgacctcccaaagtgctgggattacaagcgtgtgccaccgcacccaagggcgaattc